(19) United States
(12) Patent Application Publication
(10) Pub. No.: US 2026/0003281 A1
(43) Pub. Date: Jan. 1, 2026

(54) RESIN COMPOSITION, DIAMINE COMPOUND, CURED SUBSTANCE, LAMINATE, MANUFACTURING METHOD FOR CURED SUBSTANCE, MANUFACTURING METHOD FOR LAMINATE, MANUFACTURING METHOD FOR SEMICONDUCTOR DEVICE, AND SEMICONDUCTOR DEVICE

(71) Applicant: FUJIFILM Corporation, Tokyo (JP)

(72) Inventors: Michihiro Ogawa, Shizuoka (JP); Atsuyasu Nozaki, Shizuoka (JP)

(73) Assignee: FUJIFILM Corporation, Tokyo (JP)

(21) Appl. No.: 19/320,696

(22) Filed: Sep. 5, 2025

Related U.S. Application Data
(63) Continuation of application No. PCT/JP2024/010958, filed on Mar. 21, 2024.

(30) Foreign Application Priority Data
Mar. 24, 2023 (JP) 2023-048151

(51) Int. Cl. G03F 7/038; C07C 217/86; C08G 73/12; G03F 7/16; H01L 23/498

(52) U.S. Cl. CPC G03F 7/0388; C07C 217/86; C08G 73/127; C08G 73/128; G03F 7/0387; G03F 7/168; H01L 23/49894

(57) ABSTRACT

A resin composition containing a resin that contains a specific repeating unit and a group having an ethylenically unsaturated bond, in which a content of a phenolic hydroxyl group is 0.250 mmol/g or less, and a polymerizable compound having an ethylenically unsaturated bond, a cured substance obtained by curing the resin composition, a laminate including the cured substance, a manufacturing method for the cured substance, a manufacturing method for a laminate, a manufacturing method for a semiconductor device including the manufacturing method for a cured substance, and a semiconductor device including the cured substance, and a diamine compound having a specific structure.

### CROSS-REFERENCE TO RELATED APPLICATIONS

This application is a Continuation of PCT International Application No. PCT/JP2024/010958 filed on Mar. 21, 2024, which claims priority under 35 U.S.C § 119(a) to Japanese Patent Application No. 2023-048151 filed on Mar. 24, 2023. Each of the above application(s) is hereby expressly incorporated by reference, in its entirety, into the present application.

### BACKGROUND OF THE INVENTION

### 1. Field of the Invention

The present invention relates to a resin composition, a diamine compound, a cured substance, a laminate, a manufacturing method for a cured substance, a manufacturing method for a laminate, a manufacturing method for a semiconductor device, and a semiconductor device.

### 2. Description of the Related Art

In recent years, a resin material that has been produced from a resin composition containing a resin has been utilized in various fields.

For example, a cyclization resin such as polyimide is applied to various use applications since it has excellent heat resistance and excellent insulating properties. The above-described use applications are not particularly limited, and examples thereof in a semiconductor device for mounting include the use as a material of an insulating film or sealing material, or as a protective film. In addition, such a resin can also be used as a base film, a cover lay, or the like for a flexible substrate.

For example, in the above-described applications, the cyclization resin such as polyimide is used in the form of a resin composition containing the cyclization resin.

For example, such a resin composition is applied onto a base material by coating to form a photosensitive film, and then, as necessary, subjected to exposure, development, heating, or the like, whereby a cured substance can be formed on the base material.

Since the resin composition can be applied by a publicly known coating method or the like, it can be said that the resin composition is excellent in the manufacturing adaptability, for example, a high degree of freedom in designing the shape, size, application position of the resin composition to be applied during application. From the viewpoint of such excellent manufacturing adaptability in addition to the high performance possessed by the polyimide, the industrial application and development of the above-described resin composition are expected increasingly.

For example, JP2006-083307A describes a photosensitive polyimide siloxane and a composition including the same, which are obtained by polymerizing and imidizing (a) 85% to 99% by mole of a diamine compound having a specific structure and at least two or more photosensitive groups, (b) 15% to 1% by mole of a diamino polysiloxane having a specific structure, and (c) a tetracarboxylic acid dianhydride having a specific structure.

JP2022-135427A describes a negative-tone photosensitive resin composition containing (A) a polyimide comprising a double bond in a side chain, (B) a crosslinking agent containing a (meth)acrylate compound comprising a fluorene skeleton, and (C) a polymerization initiator.

### SUMMARY OF THE INVENTION

It is required that the cured substance obtained from the resin composition can maintain the insulating properties for a long period of time. In the present invention, the fact that the adhesiveness can be maintained for a long period of time is also referred to as “excellent reliability”.

An object of the present invention is to provide a resin composition from which a cured substance having excellent reliability is obtained, a cured substance obtained by curing the composition, a laminate including the cured substance, a manufacturing method for a cured substance, a manufacturing method for a laminate, a manufacturing method for a semiconductor device including a manufacturing method for a cured substance, and a semiconductor device including a cured substance.

In addition, another object of the present invention is to provide a novel diamine compound.

Examples of representative embodiments according to the present invention are described below.

<1> A resin composition comprising:
a resin which includes a repeating unit represented by Formula (1-1), includes a group having an ethylenically unsaturated bond, and has a content of a phenolic hydroxyl group of 0.250 mmol/g or less; and
a polymerizable compound having an ethylenically unsaturated bond,

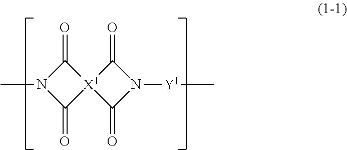

in Formula (1-1), X¹ represents a tetravalent organic group, and Y¹ represents a divalent organic group.

<2> The resin composition according to <1>,
in which the resin is a resin including a repeating unit represented by Formula (1-2) as the repeating unit represented by Formula (1-1),

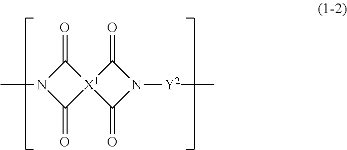

in Formula (1-2), X¹ represents a tetravalent organic group, and Y² represents a group including a group represented by Formula (2-1),

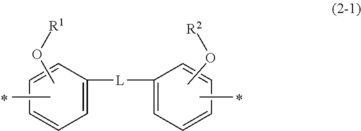

in Formula (2-1), R¹ and R² each independently represent a group having an ethylenically unsaturated bond, L represents a single bond or a divalent linking group which does not have an imide bond, and *'s each represent a bonding site to another structure.

<3> The resin composition according to <1> or <2>,
in which the resin includes a group having a vinylphenyl group as the group having an ethylenically unsaturated bond.

<4> A resin composition comprising:
a resin which includes a repeating unit represented by Formula (1-3) and a vinylphenyl group; and
a polymerizable compound having an ethylenically unsaturated bond,

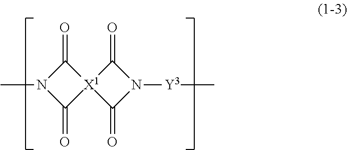

in Formula (1-3), X¹ represents a tetravalent organic group, and Y³ represents a group including a group represented by Formula (2-2),

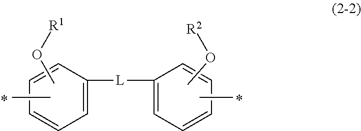

in Formula (2-2), R¹ and R² each independently represent a group having an ethylenically unsaturated bond, L represents a single bond, —C(CH₃)₂—, or —C(CF₃)₂—, and *'s each represent a bonding site to another structure.

<5> The resin composition according to any one of <1> to <4>,
in which the resin has a structure represented by any of Formulae (3-1) to (3-3),

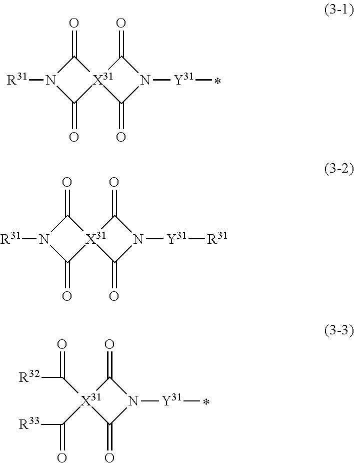

in Formula (3-1), X³¹ represents a tetravalent organic group, Y³¹ represents a divalent organic group, R³¹ represents a group having an ethylenically unsaturated bond, and * represents a bonding site to another structure,
in Formula (3-2), X³¹ represents a tetravalent organic group, Y³¹ represents a divalent organic group, R³¹ represents a group having an ethylenically unsaturated bond, and * represents a bonding site to another structure,
in Formula (3-3), X³¹ represents a tetravalent organic group, Y³¹ represents a divalent organic group, R³² and R³³ each independently represent —OH or a monovalent organic group, at least one of R³² or R³³ is a group having an ethylenically unsaturated bond, and * represents a bonding site to another structure.

<6> The resin composition according to any one of <1> to <5>,
in which the resin is a resin in which a rate of change in an imide group value calculated by Expression before and after heating at 350° C. and 1 atm for 1 hour is 25% or less,

$$rate of change ( \% ) = ( Im 2 - Im 1 ) \times 10 \; 0 / Im 1 ,$$

Im1: an imide group value before heating (mmol/g),
Im2: an imide group value (mmol/g) after heating at 350° C. and 1 atm for 1 hour.

<7> The resin composition according to any one of <1> to <6>,
in which X¹ is an organic group including a structure obtained by removing two or more hydrogen atoms from a structure represented by any of Formulae (V-1) to (V-4),

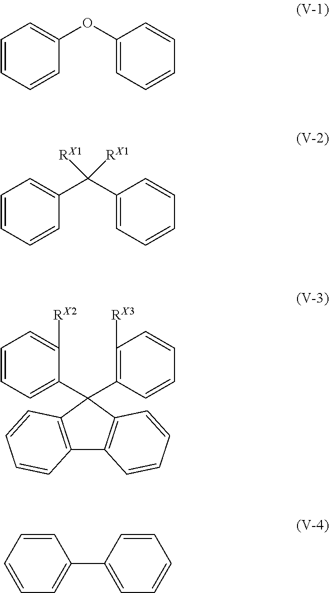

in Formula (V-2), R^{X1}'s each independently represent a hydrogen atom, an alkyl group, or a halogenated alkyl group,
in Formula (V-3), R^{X2} and R^{X3} each independently represent a hydrogen atom or a substituent, and R^{X2} and R^{X3} may be bonded to each other to form a ring structure,

<8> The resin composition according to any one of <1> to <7>, further comprising:
an azole compound.

<9> A diamine compound represented by Formula (4-1),

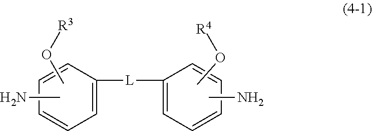

in Formula (4-1), R³ and R⁴ each independently represent a group having an ethylenically unsaturated bond, at least one of R³ or R⁴ has an aromatic hydrocarbon group, and L represents a single bond, —C(CH₃)₂—, or —C(CF₃)₂—.

<10> The diamine compound according to <9>,
in which R³ and R⁴ each independently have a vinylphenyl group.

<11> A resin composition comprising:
a resin which is a reaction product of the diamine compound according to <9> or <10> and a compound having two or more carboxy groups and carboxylic acid anhydride groups in total or a derivative thereof; and
a polymerizable compound having an ethylenically unsaturated bond.

<12> The resin composition according to any one of <1> to <11>,
in which the resin composition is used for forming an interlayer insulating film for a re-distribution layer.

<13> A cured substance obtained by curing the resin composition according to any one of <1> to <12>.

<14> A laminate comprising:
two or more layers consisting of the cured substance according to <13>; and
a metal layer provided between any layers consisting of the cured substance.

<15> A manufacturing method for a cured substance, further comprising:
a film forming step of applying the resin composition according to any one of <1> to <12> onto a base material to form a film.

<16> The manufacturing method for a cured substance according to <15>, further comprising:
an exposure step of selectively exposing the film; and
a development step of developing the film using a developer to form a pattern.

<17> The manufacturing method for a cured substance according to <15> or <16>, further comprising:
a heating step of heating the film at 50° C. to 450° C.

<18> A manufacturing method for a laminate, comprising:
the manufacturing method for a cured substance according to any one of <15> to <17>.

<19> A manufacturing method for a semiconductor device, comprising:
the manufacturing method for a cured substance according to any one of <15> to <17>.

<20> A semiconductor device comprising:
the cured substance according to <13>.

According to the present invention, there are provided a resin composition from which a cured substance having excellent reliability is obtained, a cured substance obtained by curing the composition, a laminate including the cured substance, a manufacturing method for a cured substance, a manufacturing method for a laminate, a manufacturing method for a semiconductor device including a manufacturing method for a cured substance, and a semiconductor device including a cured substance.

In addition, according to the present invention, a novel diamine compound is provided.

### DESCRIPTION OF THE PREFERRED EMBODIMENTS

Hereinafter, the main embodiments according to the present invention will be described. However, the present invention is not limited to the specified embodiments.

In the present specification, a numerical value range described by using “to” means a range including numerical values described before and after the preposition “to” as a lower limit value and an upper limit value, respectively.

In the present specification, the term “step” means not only an independent step but also a step that cannot be clearly distinguished from other steps as long as the desired action of the step can be achieved.

In describing a group (an atomic group) in the present specification, in a case where a description of substitution and unsubstitution is not provided, the description means the group includes a group (an atomic group) having a substituent as well as a group (an atomic group) having no substituent. For example, the “alkyl group” includes not only an alkyl group that does not have a substituent (an unsubstituted alkyl group) but also an alkyl group that has a substituent (a substituted alkyl group).

In the present specification, the “exposure” includes not only exposure using light but also exposure using corpuscular beams such as an electron beam and an ion beam, unless otherwise specified. In addition, examples of the light that is used for exposure include an actinic ray such as a bright line spectrum of a mercury lamp, a far ultraviolet ray represented by an excimer laser, an extreme ultraviolet ray (EUV light), an X-ray, or an electron beam, and a radioactive ray.

In the present specification, “(meth)acrylate” means one or both of “acrylate” and “methacrylate”, “(meth)acryl” means one or both of “acryl” and “methacryl”, and “(meth)acryloyl” means one or both of “acryloyl” and “methacryloyl”.

In the structural formulae of the present specification, Me represents a methyl group, Et represents an ethyl group, Bu represents a butyl group, and Ph represents a phenyl group.

In the present specification, the total solid content refers to the total mass of components excluding a solvent from the entire components of the composition. In addition, in the present specification, the concentration of solid contents is a mass percentage of other components excluding a solvent with respect to the total mass of the composition.

In the present specification, weight-average molecular weight (Mw) and number-average molecular weight (Mn) are each a value measured using gel permeation chromatography (GPC) unless otherwise specified, which are defined as a polystyrene equivalent value. In the present specification, the weight-average molecular weight (Mw) and the number-average molecular weight (Mn) can be determined, for example, by using HLC-8220 GPC (manufactured by Tosoh Corporation) and using GUARD COLUMN HZ-L, TSKgel Super HZM-M, TSK gel Super HZ4000, TSK gel Super HZ3000, and TSK gel Super HZ2000 (all of which are manufactured by Tosoh Corporation) as a column connected in series. The measurements of the above molecular weights are carried out using tetrahydrofuran (THF) as an eluent unless otherwise specified. However, N-methyl-2-pyrrolidone (NMP) can also be used in a case where THF is not suitable as the eluent, for example, in a case where the solubility is low. In addition, the detection in GPC measurement is carried out using a detector with an ultraviolet ray (a UV ray) of a wavelength of 254 nm unless otherwise specified.

In the present specification, in a case where the positional relationship of respective layers constituting the laminate is described as “upper” or “lower”, it suffices that another layer is on the upper side or the lower side of the reference layer among the plurality of layers of interest. That is, a third layer or element may be further interposed between the reference layer and the other layer, and the reference layer and the other layer need not be in contact with each other. Unless otherwise specified, the direction in which the layers are laminated on the base material is referred to as “upward”, or in a case where a resin composition layer is present, the direction from the base material to the resin composition layer is referred to as “upper”. The opposite direction thereof is referred to as “downward”. Furthermore, such a setting of upward and downward directions is for convenience in the present specification, and in a practical aspect, the “upward” direction in the present specification may be different from a vertically upward direction.

In the present specification, a composition may contain, as each component contained in the composition, two or more compounds corresponding to the component unless otherwise specified. The content of each component in the composition means the total content of all the compounds corresponding to the component unless otherwise specified.

In the present specification, unless otherwise specified, the temperature is 23° C., the atmospheric pressure is 101,325 Pa (1 atm), and the relative humidity is 50% RH.

In addition, in the present specification, a combination of preferred aspects is a more preferred aspect.

### (Resin Composition)

The resin composition according to a first aspect of the present invention (hereinafter, also simply referred to as a “first resin composition”) contains a resin which contains a repeating unit represented by Formula (1-1), contains a group having an ethylenically unsaturated bond, and has a content of a phenolic hydroxyl group of 0.250 mmol/g or less, and a polymerizable compound having an ethylenically unsaturated bond.

A resin composition according to a second aspect of the present invention (hereinafter, also simply referred to as a “second resin composition”) contains a resin containing a repeating unit represented by Formula (1-3) and a vinylphenyl group, and a polymerizable compound having an ethylenically unsaturated bond.

A resin composition according to a third aspect of the present invention (hereinafter, also simply referred to as a “third resin composition”) contains a resin which is a reaction product of a diamine compound represented by Formula (4-1) and a compound having two or more carboxy groups and carboxylic acid anhydride groups in total, and a polymerizable compound having an ethylenically unsaturated bond.

Hereinafter, the first resin composition, the second resin composition, and the third resin composition are simply referred to as a “resin composition”.

Hereinafter, the resin contained in the first resin composition, which contains a repeating unit represented by Formula (1-1), contains a group having an ethylenically unsaturated bond, and has a content of a phenolic hydroxyl group of 0.250 mmol/g or less will also be referred to as “first specific resin”.

Hereinafter, the resin which is contained in the second resin composition, includes the repeating unit represented by Formula (1-3), and includes a vinylphenyl group is also referred to as a “second specific resin”.

Hereinafter, the resin which is a reaction product of the diamine compound represented by Formula (4-1) and the compound having two or more carboxy groups and carboxylic acid anhydride groups in total, which are contained in the third resin composition, is also referred to as a “third specific resin”.

Hereinafter, in a case where the term “specific resin” is simply described, it refers to all of the first specific resin, the second specific resin, and the third specific resin.

It is preferable that the resin composition according to the embodiment of the present invention is used for forming a photosensitive film that is subjected to exposure and development, and it is more preferable that the resin composition is used for forming a film that is subjected to exposure and development using a developer containing an organic solvent.

The resin composition according to the embodiment of the present invention can be used, for example, for forming an insulating film of a semiconductor device, an interlayer insulating film for a re-distribution layer, a stress buffer film, or the like, and it is preferably used for forming an interlayer insulating film for an insulating member.

The insulating member is a member formed for the purpose of insulating members intended for conduction, such as wiring lines. The volume resistivity of the insulating member is preferably 1×10⁸ Ω·cm or more, more preferably 1×10¹⁰ Ω·cm or more, and still more preferably 1×10¹² Ω·cm or more.

In particular, one of the preferred aspects of the present invention is that the resin composition according to the embodiment of the present invention is used for forming an interlayer insulating film for a re-distribution layer.

In addition, the resin composition according to the embodiment of the present invention is preferably used for forming a photosensitive film that is subjected to negative-tone development.

In the present invention, the negative-tone development refers to development in which, in exposure and development, a non-exposed portion is removed by the development, and the positive-tone development means a development in which an exposed portion is removed by the development.

As the exposure method, the developer, and the development method, for example, the following ones in the description of the manufacturing method for a cured substance, which will be described later, are used: an exposure method to be described in the exposure step, and a developer and a development method to be described in the development step.

With the resin composition according to the embodiment of the present invention, a cured substance having excellent reliability can be obtained.

Although the mechanism by which the above effect is obtained is not revealed, it is presumed as follows.

The resin contained in the resin composition according to the first aspect has a low content of the phenolic hydroxyl group.

It is considered that the resin contained in the resin composition according to the second aspect is a resin having a vinylphenyl group, and the vinylphenyl group has lower mobility than a polymerizable group such as an acryloxy group, and thus, even in a case where the resin has a structure in which L described in Formula (2-2) is short, the resin is easily introduced into an adjacent structure.

It is considered that, in a case where a specific diamine is used as the resin contained in the resin composition according to the third aspect, the content of the phenolic hydroxyl group in the resin is lower than that in a case where a polymerizable group is introduced into a phenolic hydroxyl group having a structure derived from a diamine having a phenolic hydroxyl group.

The reason why the effect of excellent reliability is obtained by using these resins is not clear, but it is presumed that, since the content of the phenolic hydroxyl group in the resin is small, a location where the movement of the group having an ethylenically unsaturated bond in the resin is hindered by the phenolic hydroxyl group is suppressed, and the polymerization of the group having an ethylenically unsaturated bond in a portion where the phenolic hydroxyl group is present is suppressed by the polymerization inhibition effect of the phenolic hydroxyl group.

As a result, it is considered that the polymerization proceeds in a state close to uniform in the film consisting of the resin composition, and the number of unpolymerized locations in which the phenolic hydroxyl group is present is reduced in the cured substance obtained after the polymerization.

That is, in the cured substance obtained from the resin composition according to the embodiment of the present invention, it is considered that water or the like from such an unpolymerized location is suppressed from permeating, and thus the reliability is excellent.

Furthermore, it is considered that the reduction of the unpolymerized location improves the breaking elongation of the obtained cured substance.

In addition, by using a resin having a specific structure, the dielectric loss tangent of the obtained cured substance can be reduced.

In addition, in a case where the content of the phenolic hydroxyl group in the resin is small, there may be obtained an effect of improving the chemical resistance of the cured substance to be obtained, improving the developability in a case where a film consisting of the resin composition is subjected to exposure development, and the like.

Here, JP2006-083307A and JP2022-135427A do not disclose a resin composition corresponding to the resin composition according to the embodiment of the present invention.

Hereinafter, each component included in the present invention will be described in detail.

### <Specific Resin>

It is preferable that the first specific resin and the second specific resin are polyimide resins.

The polyimide resin refers to a resin including a plurality of repeating units having an imide bond.

In the present invention, the imide bond is a structure represented by —C(═O)N(—*)C(═O)—. * represents a bonding site to another structure.

In addition, the polyimide resin is preferably a resin having an imide bond in the main chain structure, and more preferably a resin having an imide ring structure in the main chain structure. The imide ring structure refers to a ring structure including all of two carbon atoms and one nitrogen atom in an imide bond as ring members. The imide ring structure is preferably a 5-membered ring structure.

In the present invention, the term “main chain” represents the relatively longest bonding chain in a resin molecule, and the term “side chain” refers to other bonding chains other than the main chain.

In addition, the third specific resin is preferably a polyimide resin, a polyamide resin, or a polyamideimide resin, and more preferably a polyimide resin.

The polyamide resin refers to a resin including a plurality of repeating units having an amide bond.

In the present invention, the amide bond is a bond represented by *—NR^{N}—C(═O)—*, where R^{N} represents a hydrogen atom or a monovalent organic group, and *'s each represent a bonding site to a carbon atom. R^{N} is preferably a hydrogen atom, an alkyl group, or an aryl group, and more preferably a hydrogen atom.

The polyamideimide resin refers to a resin including a plurality of repeating units having an imide bond and an amide bond.

### [Content of Phenolic Hydroxyl Group]

The content of the phenolic hydroxyl group in the first specific resin is 0.250 mmol/g or less.

In the second specific resin and the third specific resin, the content of the phenolic hydroxyl group is preferably 0.250 mmol/g or less.

In the present invention, the phenolic hydroxy group refers to a hydroxy group that is bonded to an aromatic ring structure by a single bond without a linking group. The above-described aromatic ring structure may be an aromatic hydrocarbon ring structure or an aromatic heterocyclic structure.

The content of the phenolic hydroxyl group in the specific resin is preferably 0.200 mmol/g or less, more preferably 0.180 mmol/g or less, still more preferably 0.150 mmol/g or less, and particularly preferably 0.120 mmol/g or less.

Here, the lower limit of the content of the phenolic hydroxyl group is not particularly limited, and may be 0 mmol/g (that is, equal to or less than the limit of quantification).

The content of the phenolic hydroxyl group can be calculated by dividing the amount (mmol) of the phenolic hydroxyl group measured by ¹H-NMR with respect to 1 mol of the resin by the number-average molecular weight of the resin.

The amount (mmol) of the phenolic hydroxyl group can be calculated by known quantitative NMR.

Specifically, the measurement can be performed under the following measurement conditions.

### —Measurement Conditions—

¹H-NMR (BRUKER, AVANCE NEO 400)
Analysis software: TopSpin 4.0.7
Solvent: DMSO-d6
Internal standard: 1,3,5-trimethoxybenzene

In ¹H-NMR, a proton of a phenolic hydroxyl group is detected as a specific peak near δ (DMSO-d6)=10 ppm. In addition, the internal standard 1,3,5-trimethoxybenzene is detected at a specific peak at 6.1 ppm.

The number-average molecular weight is obtained as a polystyrene-equivalent value measured by gel permeation chromatography (GPC).

In addition, in a case where the above measurement is difficult, the acid value (mgKOH/g) of the resin is measured by acid value titration, the acid value is converted into mol/g, the proportion of the phenolic hydroxyl group among the acid groups contained in the specific resin is quantified by NMR, and the quantified value is integrated into the acid value, whereby the acid value can also be calculated.

In the acid value titration, the neutralization points can be distinguished and detected because there is a difference in pKa between the carboxylic acids.

### [Group Having Ethylenically Unsaturated Bond]

The first specific resin includes a group having an ethylenically unsaturated bond.

The first specific resin may have a group having an ethylenically unsaturated bond in any portion of the resin, but the group having an ethylenically unsaturated bond is preferably contained in the repeating unit represented by Formula (1-1), and more preferably contained in Y¹ in the repeating unit represented by Formula (1-1).

Examples of the group having an ethylenically unsaturated bond include a group including at least one group selected from the group consisting of a vinyl group, an allyl group, an isoallyl group, a 2-methylallyl group, a group having an aromatic ring directly bonded to a vinyl group (for example, a vinylphenyl group), a (meth)acrylamide group, and a (meth)acryloyloxy group.

In the present invention, the direct bonding of a certain structure and another structure to each other refers to bonding of a certain structure and another structure to each other without a linking group.

From the viewpoint of reactivity, the first specific resin preferably includes, as a group having an ethylenically unsaturated bond, a group having an aromatic ring directly bonded to a vinyl group, and a group having at least one group selected from the group consisting of a (meth)acrylamide group and a (meth)acryloyloxy group.

From the viewpoint of reducing the dielectric loss tangent, the first specific resin preferably includes a group having an aromatic ring directly bonded to a vinyl group as a group having an ethylenically unsaturated bond, and more preferably includes a group having a vinylphenyl group.

The content of the group having an ethylenically unsaturated bond in the specific resin is preferably 0.1 to 3.0 mmol/g, more preferably 0.15 to 2.75 mmol/g, and still more preferably 0.2 to 2.5 mmol/g.

In addition, in a case where the specific resin contains a vinylphenyl group, the content of the vinylphenyl group in the specific resin is preferably 0.1 to 3.0 mmol/g, more preferably 0.15 to 2.75 mmol/g, and still more preferably 0.2 to 2.5 mmol/g.

### [Repeating Unit Represented by Formula (1-1)]

The first specific resin includes a repeating unit represented by Formula (1-1).

The third specific resin preferably includes a repeating unit represented by Formula (1-1). In that case, X¹ in Formula (1-1) is a structure derived from a specific carboxylic acid compound described later, and Y¹ is a structure derived from the diamine compound according to the embodiment of the present invention.

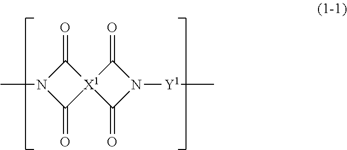

In Formula (1-1), X¹ represents a tetravalent organic group, and Y¹ represents a divalent organic group.

### —X¹—

In Formula (1-1), X¹ preferably represents an organic group including a structure obtained by removing two or more hydrogen atoms from a structure represented by any of Formulae (V-1) to (V-9), and more preferably represents an organic group including a structure obtained by removing two or more hydrogen atoms from a structure represented by any of Formulae (V-1) to (V-4).

By being an organic group including a structure obtained by removing two or more hydrogen atoms from a structure represented by any of Formula (V-1) to Formula (V-9), chemical resistance and flatness of the cured substance are improved.

Here, the organic group is an organic group including a structure obtained by removing two or more hydrogen atoms from a structure represented by any of Formula (V-1) to Formula (V-4), and thus, effects such as suppression of generation of development residues, reduction of the dielectric constant of the cured substance, and reduction of the coefficient of thermal expansion can also be obtained.

Here, since the organic group is an organic group including a structure obtained by removing two or more hydrogen atoms from a structure represented by any of Formula (V-5) to Formula (V-9), effects such as that the pattern of the cured substance is less likely to be tapered due to improvement in the transmittance of ultraviolet light and that the tolerance to the exposure amount is wide can also be obtained.

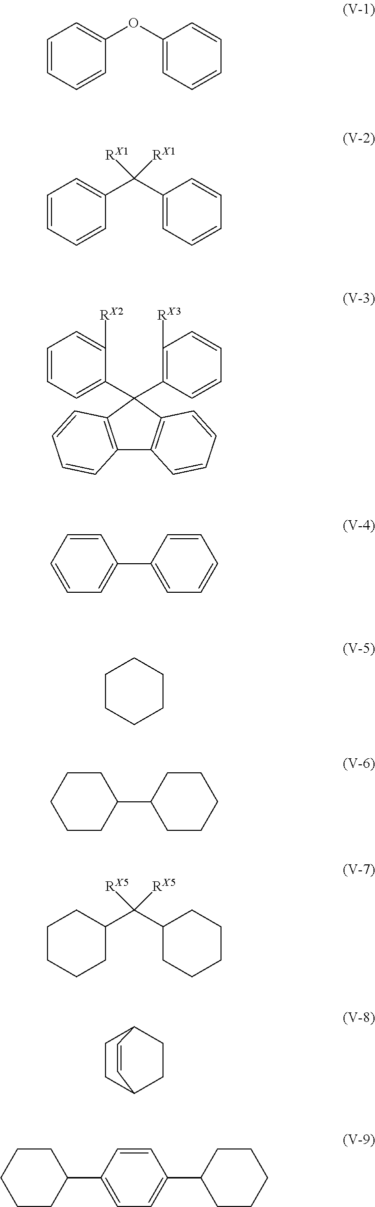

In Formula (V-2), R^{X1}'s each independently represent a hydrogen atom, an alkyl group, or a halogenated alkyl group.

In Formula (V-3), R^{X2} and R^{X3} each independently represent a hydrogen atom or a substituent, and R^{X2} and R^{X3} may be bonded to each other to form a ring structure.

In Formula (V-7), R^{X5}'s each independently represent a hydrogen atom, an alkyl group, or a halogenated alkyl group.

In Formula (V-2), R^{X1}'s are each independently preferably an alkyl group or a halogenated alkyl group, more preferably an alkyl group having 1 to 4 carbon atoms or a halogenated alkyl group having 1 to 4 carbon atoms, and still more preferably a methyl group or a trifluoromethyl group. The halogenated alkyl group refers to a group in which at least one hydrogen atom of an alkyl group is substituted with a halogen atom. As the halogen atom, F or Cl is preferable, and F is more preferable.

In Formula (V-3), R^{X2} and R^{X3} are each independently preferably a hydrogen atom.

In a case where R^{X2} and R^{X3} are bonded to each other to form a ring structure, the structure formed by the bonding of R^{X2} and R^{X3} is preferably a single bond, —O—, or —C(R)₂—, more preferably —O— or —C(R)₂—, and still more preferably —O—. R represents a hydrogen atom or a monovalent organic group, preferably a hydrogen atom, an alkyl group, or an aryl group, and more preferably a hydrogen atom.

In Formula (V-7), R^{X5}'s are each independently preferably an alkyl group or a halogenated alkyl group, more preferably an alkyl group having 1 to 4 carbon atoms or a halogenated alkyl group having 1 to 4 carbon atoms, and still more preferably a methyl group or a trifluoromethyl group. The halogenated alkyl group refers to a group in which at least one hydrogen atom of an alkyl group is substituted with a halogen atom. As the halogen atom, F or Cl is preferable, and F is more preferable.

In a case where X¹ is a group including a structure obtained by removing two or more hydrogen atoms from a structure represented by Formula (V-1), X¹ is preferably a group represented by Formula (V-1-1). In the following Formula, * represents a bonding site to four carbonyl groups to which X¹ in Formula (1-1) is bonded, and n1 represents an integer of 0 to 5, and it is also preferable that n1 is an integer of 1 to 5. In addition, the hydrogen atom in the following structure may be further substituted with a known substituent such as a hydrocarbon group.

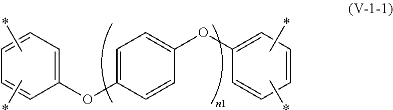

In a case where X¹ is a group including a structure obtained by removing two or more hydrogen atoms from a structure represented by Formula (V-2), X¹ is preferably a group represented by Formula (V-2-1) or Formula (V-2-2), and from the viewpoint of reducing the amine value in the resin, X¹ is preferably a group represented by Formula (V-2-2). In the present specification, a bond that intersects with a side of a ring structure means that any hydrogen atom in the ring structure is substituted. In the following Formula, L^{X1} represents a single bond or —O—, and * represents a bonding site to four carbonyl groups to which X¹ in Formula (1-1) is bonded. In addition, the definition and preferred aspect of R^{X1} are as described above. In addition, the hydrogen atom in these structures may be further substituted with a known substituent such as a hydrocarbon group.

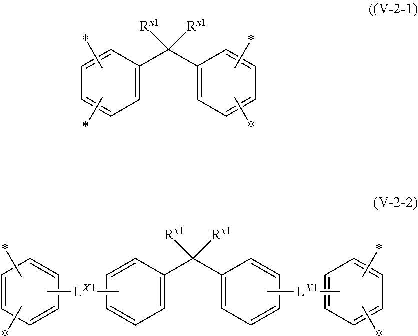

In a case where X¹ is a group including a structure obtained by removing two or more hydrogen atoms from a structure represented by Formula (V-3), X¹ is preferably a group represented by Formula (V-3-1) or Formula (V-3-2), and from the viewpoint of reducing the dielectric constant of the cured substance or the like, X¹ is preferably a group represented by Formula (V-3-2). In the following Formula, * represents a bonding site to four carbonyl groups to which X¹ in Formula (1-1) is bonded. In addition, the definitions and preferred aspects of R^{X2} and R^{X3} are as described above. In addition, the hydrogen atom in these structures may be further substituted with a known substituent such as a hydrocarbon group.

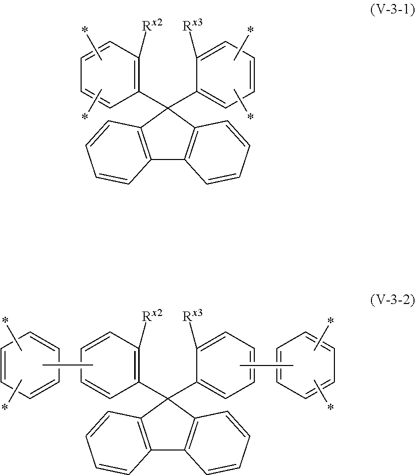

In a case where X¹ is a group including a structure obtained by removing two or more hydrogen atoms from a structure represented by Formula (V-4), X¹ is preferably a group represented by Formula (V-4-1). In the following Formula, * represents a bonding site to four carbonyl groups to which X¹ in Formula (1-1) is bonded, and n1 represents an integer of 0 to 5. In addition, the hydrogen atom in the following structure may be further substituted with a known substituent such as a hydrocarbon group. However, it is also preferable that none of the hydrogen atoms in the structure represented by (V-4-1) are substituted.

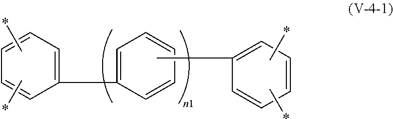

In a case where X¹ is a group including a structure obtained by removing two or more hydrogen atoms from a structure represented by Formula (V-5), X¹ is preferably a group represented by Formula (V-5-1). In the following Formula, * represents a bonding site to four carbonyl groups to which X¹ in Formula (1-1) is bonded. In addition, the hydrogen atom in the following structure may be further substituted with a known substituent such as a hydrocarbon group.

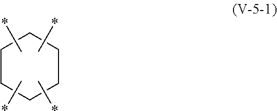

In a case where X¹ is a group including a structure obtained by removing two or more hydrogen atoms from a structure represented by Formula (V-6), X¹ is preferably a group represented by Formula (V-6-1). In the following Formula, * represents a bonding site to four carbonyl groups to which X¹ in Formula (1-1) is bonded. In addition, the hydrogen atom in the following structure may be further substituted with a known substituent such as a hydrocarbon group.

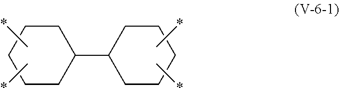

In a case where X¹ is a group including a structure obtained by removing two or more hydrogen atoms from a structure represented by Formula (V-7), X¹ is preferably a group represented by Formula (V-7-1). In the following Formula, * represents a bonding site to four carbonyl groups to which X¹ in Formula (1-1) is bonded. In addition, the hydrogen atom in the following structure may be further substituted with a known substituent such as a hydrocarbon group.

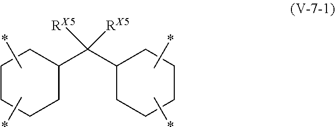

In a case where X¹ is a group including a structure obtained by removing two or more hydrogen atoms from a structure represented by Formula (V-8), X¹ is preferably a group represented by Formula (V-8-1). In the following Formula, * represents a bonding site to four carbonyl groups to which X¹ in Formula (1-1) is bonded. In addition, the hydrogen atom in the following structure may be further substituted with a known substituent such as a hydrocarbon group.

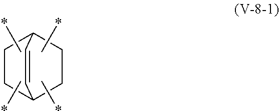

In a case where X¹ is a group including a structure obtained by removing two or more hydrogen atoms from a structure represented by Formula (V-9), X¹ is preferably a group represented by Formula (V-9-1). In the following Formula, * represents a bonding site to four carbonyl groups to which X¹ in Formula (1-1) is bonded. In addition, the hydrogen atom in the following structure may be further substituted with a known substituent such as a hydrocarbon group.

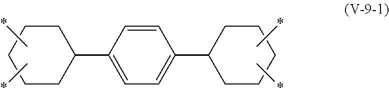

In addition, X¹ may be a tetracarboxylic acid residue remaining after the removal of the anhydride group from the tetracarboxylic acid dianhydride described in paragraphs 0055 to 0057 of JP2023-003421A.

In addition, it is preferable that X¹ does not contain an imide bond in the structure.

In addition, it is preferable that X¹ does not contain a urethane bond, a urea bond, and an amide bond in the structure.

In the present invention, the urethane bond is a bond represented by *—O—C(═O)—NR^{N}—*, where R^{N} represents a hydrogen atom or a monovalent organic group, and *'s each represent a bonding site to a carbon atom. The preferred aspect of R^{N} is as described above.

In the present invention, the urea bond is a bond represented by *—NR^{N}—C(═O)—NR^{N}—*, where R^{N}'s each independently represent a hydrogen atom or a monovalent organic group, and *'s each represent a bonding site to a carbon atom. The preferred aspect of R^{N} is as described above.

Furthermore, it is preferable that X¹ does not contain an ester bond in the structure.

In the present invention, the ester bond is a bond represented by *—O—C(═O)—*.

Among these, it is preferable that X¹ does not contain an imide bond, a urethane bond, a urea bond, and an amide bond, and it is preferable that X¹ does not contain an imide bond, a urethane bond, a urea bond, an amide bond, and an ester bond.

### —Y¹—

In Formula (1-1), Y¹ is preferably a group including a group represented by Formula (2-1), and more preferably a group represented by Formula (2-1).

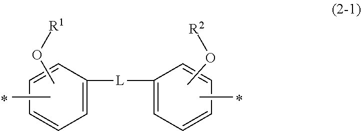

In Formula (2-1), R¹ and R² each independently represent a group having an ethylenically unsaturated bond, L represents a single bond or a divalent linking group which does not have an imide bond, and *'s each represent a bonding site to another structure.

In Formula (2-1), R¹ and R² are each independently preferably a group represented by Formula (R1-1).

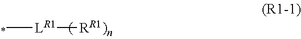

In Formula (R1-1), L^{R1} represents an (n+1)-valent linking group, R^{R1}'s each independently represent an aromatic group directly bonded to a vinyl group, a (meth)acryloxy group, or a (meth)acrylamide group, n represents an integer of 1 to 10, and * represents a bonding site to an oxygen atom in Formula (2-1).

R^{R1}'s are each independently preferably an aromatic group directly bonded to a vinyl group, and more preferably a vinylphenyl group.

L^{R1} is preferably a hydrocarbon group or a group represented by a bond between a hydrocarbon group and at least one group selected from the group consisting of —O—, —C(═O)—, —S—, —S(═O)₂—, and —NR^{N}—, and it is preferably a hydrocarbon group or a group represented by *¹—C(═O)NR^{N}-L^{R2}-*².

The above-described hydrocarbon group is preferably an alkylene group, more preferably an alkylene group having 1 to 10 carbon atoms, and still more preferably an alkylene group having 1 to 4 carbon atoms.

The preferred aspect of R^{N} is as described above.

*¹ has the same definition as * in Formula (R1-1), and *² represents a bonding site to R^{R1} in Formula (R1-1).

In addition, in a case where R^{R1} is a vinylphenyl group, L^{R1} is preferably an alkylene group having 1 to 4 carbon atoms, and more preferably a methylene group.

In a case where R^{R1} is a (meth)acryloxy group or a (meth)acrylamide group, L^{R1} is preferably a group represented by *¹—C(═O)NR^{N}-L^{R2}-*².

n is preferably an integer of 1 to 4, preferably 1 or 2, and more preferably 1.

In Formula (2-1), L is preferably a single bond, —C(CH₃)₂—, —C(CF₃)₂—, —S(═O)₂—, or a 9,9-fluorenyl group. In addition, an aspect in which L is a single bond, —C(CH₃)₂—, or —C(CF₃)₂— is also one of the preferred aspects of the present invention.

In addition, in Formula (1-1), Y¹ may be a group including a structure obtained by removing two or more hydrogen atoms from the structure represented by any of Formula (V-1) to Formula (V-9) described above.

By being an organic group including a structure obtained by removing two or more hydrogen atoms from a structure represented by any of Formula (V-1) to Formula (V-9), chemical resistance and flatness of the cured substance are improved.

In a case where Y¹ is a group including a structure obtained by removing two or more hydrogen atoms from a structure represented by Formula (V-1), Y¹ is preferably a group represented by Formula (V-1-2). In the following Formula, * represents a bonding site to two nitrogen atoms to which Y¹ in Formula (1-1) is bonded, and n1 represents an integer of 1 to 5. In addition, the hydrogen atom in the following structure may be further substituted with a known substituent such as a hydrocarbon group.

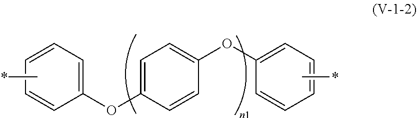

In a case where Y¹ is a group including a structure obtained by removing two or more hydrogen atoms from a structure represented by Formula (V-2), Y¹ is preferably a group represented by Formula (V-2-3) or Formula (V-2-4), and from the viewpoint of reducing the dielectric constant of the cured substance or the like, Y¹ is preferably a group represented by Formula (V-2-4). In the following Formula, L^{X1} represents a single bond or —O—, and * represents a bonding site to two nitrogen atoms to which Y¹ in Formula (1-1) is bonded. In addition, the preferred aspect of R^{X1} is as described above. In addition, the hydrogen atom in these structures may be further substituted with a known substituent such as a hydrocarbon group.

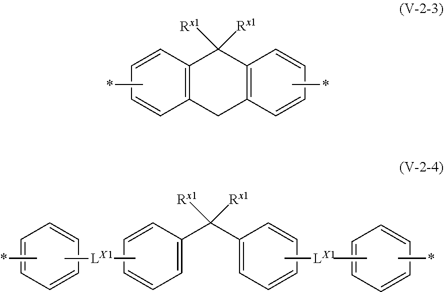

In a case where Y¹ is a group including a structure obtained by removing two or more hydrogen atoms from a structure represented by Formula (V-3), Y¹ is preferably a group represented by Formula (V-3-3) or Formula (V-3-4), and from the viewpoint of reducing the dielectric constant of the cured substance or the like, Y¹ is preferably a group represented by Formula (V-3-3). In the following Formula, * represents a bonding site to two nitrogen atoms to which Y¹ in Formula (1-1) is bonded. In addition, the hydrogen atom in these structures may be further substituted with a known substituent such as a hydrocarbon group.

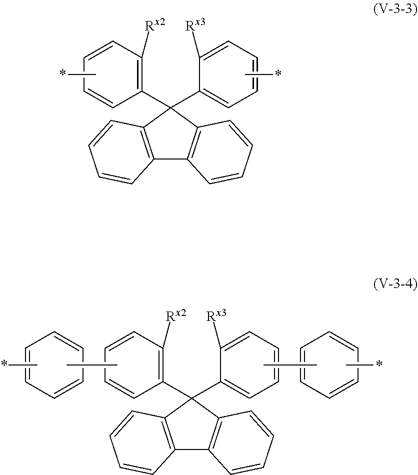

In a case where Y¹ is a group including a structure obtained by removing two or more hydrogen atoms from a structure represented by Formula (V-4), Y¹ is preferably a group represented by Formula (V-4-2). In the following Formula, * represents a bonding site to two nitrogen atoms to which Y¹ in Formula (1-1) is bonded, and n1 represents an integer of 0 to 5. In addition, an aspect in which n1 is 0 is also one of the preferred aspects of the present invention. In addition, the hydrogen atom in the following structure may be further substituted with a known substituent such as a hydrocarbon group.

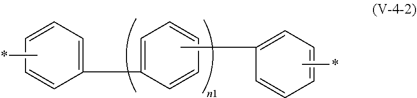

In a case where Y¹ is a group including a structure obtained by removing two or more hydrogen atoms from a structure represented by Formula (V-5), Y¹ is preferably a group represented by Formula (V-5-2). In the following Formula, * represents a bonding site to two nitrogen atoms to which Y¹ in Formula (1-1) is bonded. In addition, the hydrogen atom in the following structure may be further substituted with a known substituent such as a hydrocarbon group.

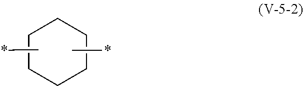

In a case where Y¹ is a group including a structure obtained by removing two or more hydrogen atoms from a structure represented by Formula (V-6), Y¹ is preferably a group represented by Formula (V-6-2). In the following Formula, * represents a bonding site to two nitrogen atoms to which Y¹ in Formula (1-1) is bonded. In addition, the hydrogen atom in the following structure may be further substituted with a known substituent such as a hydrocarbon group.

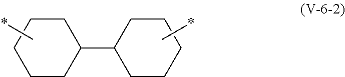

In a case where Y¹ is a group including a structure obtained by removing two or more hydrogen atoms from a structure represented by Formula (V-7), Y¹ is preferably a group represented by Formula (V-7-2). In the following Formula, * represents a bonding site to two nitrogen atoms to which Y¹ in Formula (1-1) is bonded. In addition, the hydrogen atom in the following structure may be further substituted with a known substituent such as a hydrocarbon group.

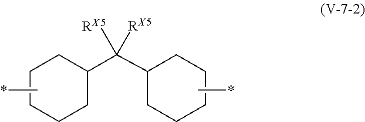

In a case where Y¹ is a group including a structure obtained by removing two or more hydrogen atoms from a structure represented by Formula (V-8), Y¹ is preferably a group represented by Formula (V-8-2). In the following Formula, * represents a bonding site to two nitrogen atoms to which Y¹ in Formula (1-1) is bonded. In addition, the hydrogen atom in the following structure may be further substituted with a known substituent such as a hydrocarbon group.

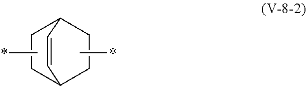

In a case where Y¹ is a group including a structure obtained by removing two or more hydrogen atoms from a structure represented by Formula (V-9), Y¹ is preferably a group represented by Formula (V-9-2). In the following Formula, * represents a bonding site to two nitrogen atoms to which Y¹ in Formula (1-1) is bonded. In addition, the hydrogen atom in the following structure may be further substituted with a known substituent such as a hydrocarbon group.

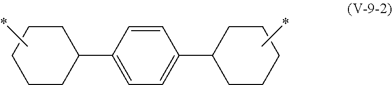

In addition, Y¹ may be a group described in paragraphs 0042 to 0053 of JP2023-003421A.

In addition, it is preferable that Y¹ does not contain an imide bond in the structure.

In addition, it is preferable that Y¹ does not contain a urethane bond, a urea bond, and an amide bond in the structure.

Furthermore, it is preferable that Y¹ does not contain an ester bond in the structure.

Among these, it is preferable that Y¹ does not contain an imide bond, a urethane bond, a urea bond, and an amide bond, and it is preferable that Y¹ does not contain an imide bond, a urethane bond, a urea bond, an amide bond, and an ester bond.

### [Repeating Unit Represented by Formula (1-2)]

It is preferable that the first specific resin includes a repeating unit represented by Formula (1-2) as the repeating unit represented by Formula (1-1).

The third specific resin preferably includes a repeating unit represented by Formula (1-2). In that case, X¹ in Formula (1-2) is a structure derived from a specific carboxylic acid compound described later, and Y² is a structure derived from the diamine compound according to the embodiment of the present invention.

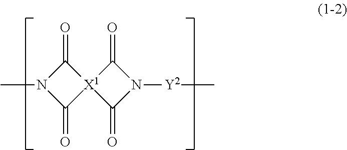

In Formula (1-2), X¹ represents a tetravalent organic group, and Y² represents a group including a group represented by Formula (2-1).

In Formula (1-2), a preferred aspect of X¹ is the same as the preferred aspect of X¹ in Formula (1-1) described above.

In Formula (1-2), a preferred aspect of the group including the group represented by Formula (2-1) in Y² is the same as the preferred aspect of the group including the group represented by Formula (2-1) in Y¹ in Formula (1-1) described above.

### [Repeating Unit Represented by Formula (1-3)]

The second resin composition contains a repeating unit represented by Formula (1-3).

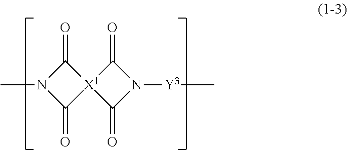

In Formula (1-3), X¹ represents a tetravalent organic group, and Y³ represents a group including a group represented by Formula (2-2).

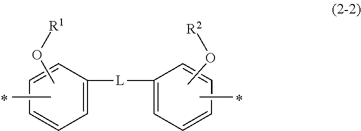

In Formula (2-2), R¹ and R² each independently represent a group having an ethylenically unsaturated bond, L represents a single bond, —C(CH₃)₂—, or —C(CF₃)₂—, and *'s each represent a bonding site to another structure.

In Formula (1-3), a preferred aspect of X¹ is the same as the preferred aspect of X¹ in Formula (1-1).

In Formula (2-2), preferred aspects of R¹ and R² are the same as the preferred aspects of R¹ and R² in Formula (2-1).

### [Structures Represented by Formulae (3-1) to (3-3)]

The specific resin preferably has a structure represented by any of Formulae (3-1) to (3-3).

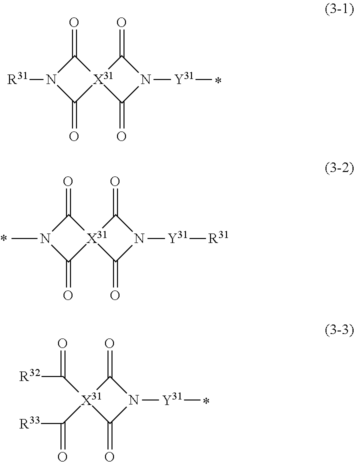

In Formula (3-1), X³¹ represents a tetravalent organic group, Y³¹ represents a divalent organic group, R³¹ represents a group having an ethylenically unsaturated bond, and * represents a bonding site to another structure.

In Formula (3-2), X³¹ represents a tetravalent organic group, Y³¹ represents a divalent organic group, R³¹ represents a group having an ethylenically unsaturated bond, and * represents a bonding site to another structure.

In Formula (3-3), X³¹ represents a tetravalent organic group, Y³¹ represents a divalent organic group, R³² and R³³ each independently represent —OH or a monovalent organic group, at least one of R³² or R³³ is a group having an ethylenically unsaturated bond, and * represents a bonding site to another structure.

In Formulae (3-1) to (3-3), preferred aspects of X³¹ and Y³¹ are the same as the preferred aspects of X¹ and Y¹ in Formula (1-1) described above.

In Formulae (3-1) to (3-3), R³¹ to R³³ preferably include a group having an ethylenically unsaturated bond, more preferably include a group having an aromatic ring directly bonded to a vinyl group, and still more preferably include a group having a vinylphenyl group.

In addition, in Formulae (3-1) to (3-3), it is preferable that R³¹ to R³³ do not contain an imide bond.

Among these, it is preferable that the specific resin includes a structure represented by Formula (3-1), and it is more preferable that the specific resin is represented by Formula (3-1) and R³¹ is a group represented by Formula (R3-1).

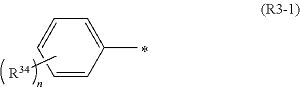

In Formula (R3-1), R³⁴ represents a substituent, n represents an integer of 0 to 5, and * represents a bonding site to a nitrogen atom in Formula (3-1).

R³⁴ is preferably an alkyl group, an aryl group, or a group having an ethylenically unsaturated bond, and it is preferably an alkyl group or a vinylphenylmethyl group.

n is preferably 0 or 1, and more preferably 1.

### [Reaction Product]

The third specific resin is a reaction product of the diamine compound according to the embodiment of the present invention and a compound having two or more carboxy groups and carboxylic acid anhydride groups in total (also referred to as a “specific carboxylic acid compound”) or a derivative thereof.

Here, in a case where the reaction for obtaining the reaction product is allowed to proceed by the chemical imidization, the third specific resin is preferably a reaction product of the diamine compound according to the embodiment of the present invention and the specific carboxylic acid compound.

It is preferable that the first specific resin and the second specific resin are reaction products of the diamine compound according to the embodiment of the present invention and a compound having two carboxylic acid anhydride groups or a derivative thereof.

The diamine compound according to the embodiment of the present invention will be described in detail later.

As described later, the diamine compound according to the embodiment of the present invention includes a group having an ethylenically unsaturated bond.

Here, for example, in a case where the group represented by Formula (2-1) is to be introduced into the specific resin, it is also conceivable to introduce a structure having a phenolic hydroxyl group as Y¹ in Formula (1-1) as in Formula (2-1C), and then react a compound which includes a group having an ethylenically unsaturated bond and reacts with a phenolic hydroxyl group, such as an isocyanate compound including a group having an ethylenically unsaturated bond, with the phenolic hydroxyl group to form R¹ and R² in Formula (2-1).

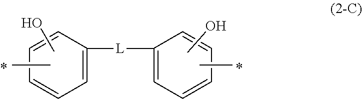

In Formula (2-C), L has the same definition as L in Formula (2-1) described above, and the same applies to the preferred aspect thereof.

However, according to such an aspect, the phenolic hydroxyl group may remain in the resin, and the introduction amount of the group having an ethylenically unsaturated bond may vary.

In a case where the introduction amount varies in this way, there is a case where variation in breaking elongation or the like of the cured film occurs, or the dielectric loss tangent of the cured film increases due to the residual of the phenolic hydroxyl group.

Since the diamine according to the embodiment of the present invention includes a group having an ethylenically unsaturated bond, by using the diamine in a reaction with the specific carboxylic acid compound, it is easy to introduce a group represented by Formula (2-1) into the resin, and the residual of the phenolic hydroxyl group in the resin is suppressed. As a result, it is considered that the effect of suppressing the variation in the breaking elongation of the cured substance and the effect of reducing the dielectric loss tangent are obtained.

Examples of the specific carboxylic acid compound include a compound having two carboxy groups, a compound having one carboxy group and one carboxylic acid anhydride group, and a compound having two carboxylic acid anhydride groups, and a compound having two carboxylic acid anhydride groups (that is, carboxylic acid dianhydride) is preferable.

By using a compound having two carboxylic acid anhydride groups or a derivative thereof, the specific resin which is a polyimide is obtained.

Examples of the derivative of the compound having two carboxylic acid anhydride groups include a diester of a compound having two carboxylic acid anhydride groups and a diester dihalide compound obtained from a compound having two carboxylic acid anhydride groups.

The diester is obtained, for example, by reacting a compound having two carboxylic acid anhydride groups with an alkylating agent or an alcohol.

Examples of the alkylating agent include N,N-dimethylformamide dimethyl acetal, N,N-dimethylformamide diethyl acetal, N,N-dialkylformamide dialkyl acetal, trimethyl orthoformate, triethyl orthoformate.

Examples of the above-described alcohol include a compound represented by R^{C1}—OH. R^{C1} has the same definition as R^{C1} in Formula (C1-2) described later, and the same applies to the preferred aspect thereof.

The above-described diester dihalide compound can be obtained, for example, by reacting the above-described diester with a halogenating agent.

Examples of the halogenating agent include thionyl chloride, oxalyl chloride, and phosphorus oxychloride.

Examples of the compound having two carboxylic acid anhydride groups or a derivative thereof include compounds represented by Formulae (C1-1) to (C1-3), but the present invention is not limited thereto.

Here, in a case where the reaction for obtaining the reaction product is allowed to proceed by the chemical imidization, the compound represented by Formula (C1-1) is preferable.

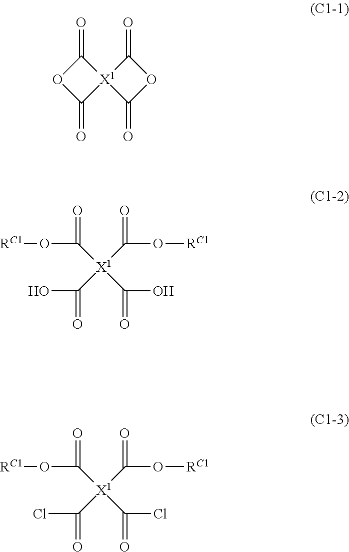

In Formula (C1-1), X¹ represents a tetravalent organic group.

In Formula (C1-2), X¹ represents a tetravalent organic group, and R^{C1}'s each independently represent a monovalent organic group.

In Formula (C1-3), X¹ represents a tetravalent organic group, and R^{C1}'s each independently represent a monovalent organic group.

In Formulae (C1-1) to (C1-3), a preferred aspect of X¹ is the same as the preferred aspect of X¹ in Formula (1-1).

In Formulae (C1-2) and (C1-3), the preferred aspects of R^{C1} are the same as the preferred aspects of R¹¹³ and R¹¹⁴ described in paragraphs 0058 to 0063 of JP2023-003421A.

By using a compound having one carboxy group and one carboxylic acid anhydride group, the specific resin which is a polyamideimide is obtained.

By using a compound having two carboxylic acid anhydride groups, a specific resin which is a polyamide is obtained.

### [Details of Reaction for Obtaining Reaction Product]

In the reaction for obtaining the reaction product, it is preferable to use an organic solvent at the time of the reaction. One kind of organic solvent may be used, or two or more kinds thereof may be used.

The organic solvent can be appropriately determined depending on the raw material; however, examples thereof include pyridine, diethylene glycol dimethyl ether (diglyme), N-methylpyrrolidone, N-ethylpyrrolidone, ethyl propionate, dimethylacetamide, dimethylformamide, tetrahydrofuran, and γ-butyrolactone.

In the reaction for obtaining the reaction product, it is preferable to add a basic compound at the time of the reaction. One kind of basic compound may be used, or two or more kinds thereof may be used.

The basic compound can be appropriately determined depending on the raw material; however, examples thereof include triethylamine, diisopropylethylamine, pyridine, 1,8-diazabicyclo[5.4.0]undec-7-ene, and N,N-dimethyl-4-aminopyridine.

In addition, in a case where the reaction product is a compound having an imide bond, the imidization can also be promoted (chemical imidization) by using a dehydration cyclization reagent such as a combination of a carboxylic acid anhydride and an amine in the reaction for obtaining the reaction product. As the dehydration cyclization reagent, for example, a reagent known in the field of chemical imidization, such as a combination of acetic anhydride and pyridine, can be used.

By performing the chemical imidization, the imidization reaction can proceed at a low temperature, and the ring-opening portion is less likely to remain. Therefore, there are advantages such as suppression of oxidation coloration of amine, difficulty in remaining of a carboxy group or an amide group due to difficulty in remaining of a ring-opening portion, improvement of reliability of a cured substance, and decrease in dielectric loss tangent.

In addition, since the diamine compound according to the embodiment of the present invention contains a group having an ethylenically unsaturated bond, it is preferable to add a polymerization inhibitor in the reaction for obtaining a reaction product. Examples of the polymerization inhibitor include a polymerization inhibitor contained in the resin composition according to the embodiment of the present invention, which will be described later.

In the reaction for obtaining the reaction product, it is also preferable to use a terminal blocking material. Examples of the terminal blocking agent include a monoalcohol, phenol, thiol, thiophenol, and a monoamine. It is more preferable to use a monoalcohol, phenols, or a monoamine from the viewpoint of reactivity and film stability. Examples of the preferred monoalcohol compound include primary alcohol such as methanol, ethanol, propanol, butanol, hexanol, octanol, dodecanol, benzyl alcohol, 2-phenylethanol, 2-methoxyethanol, 2-chloromethanol, and furfuryl alcohol; secondary alcohol such as isopropanol, 2-butanol, cyclohexyl alcohol, cyclopentanol, and 1-methoxy-2-propanol; and tertiary alcohol such as t-butyl alcohol and adamantane alcohol. Examples of the preferred compounds of phenols include phenols such as phenol, methoxyphenol, methylphenol, naphthalene-1-ol, naphthalene-2-ol, and hydroxystyrene. In addition, examples of the preferred monoamine compound include aniline, 2-ethynyl aniline, 3-ethynyl aniline, 4-ethynyl aniline, 5-amino-8-hydroxyquinoline, 1-hydroxy-7-aminonaphthalene, 1-hydroxy-6-aminonaphthalene, 1-hydroxy-5-aminonaphthalene, 1-hydroxy-4-aminonaphthalene, 2-hydroxy-7-aminonaphthalene, 2-hydroxy-6-aminonaphthalene, 2-hydroxy-5-aminonaphthalene, 1-carboxy-7-aminonaphthalene, 1-carboxy-6-aminonaphthalene, 1-carboxy-5-aminonaphthalene, 2-carboxy-7-aminonaphthalene, 2-carboxy-6-aminonaphthalene, 2-carboxy-5-aminonaphthalene, 2-aminobenzoic acid, 3-aminobenzoic acid, 4-aminobenzoic acid, 4-aminosalicylic acid, 5-aminosalicylic acid, 6-aminosalicylic acid, 2-aminobenzenesulfonic acid, 3-aminobenzenesulfonic acid, 4-aminobenzenesulfonic acid, 3-amino-4,6-dihydroxypyrimidine, 2-aminophenol, 3-aminophenol, 4-aminophenol, 2-aminothiophenol, 3-aminothiophenol, and 4-aminothiophenol. Two or more of these may be used, and a plurality of different terminal groups may be introduced by reacting a plurality of terminal blocking agents.

In addition, in a case of blocking the amino group at the terminal of the resin, it is possible to carry out blocking with a compound having a functional group capable of reacting with the amino group. The preferred terminal blocking agent for the amino group is preferably a carboxylic acid anhydride, a carboxylic acid chloride, a carboxylic acid bromide, a sulfonic acid chloride, sulfonic acid anhydride, or a sulfonic acid carboxylic acid anhydride, and more preferably a carboxylic acid anhydride or a carboxylic acid chloride. Examples of the preferred carboxylic acid anhydride compound include acetic anhydride, propionic anhydride, oxalic anhydride, succinic anhydride, maleic acid anhydride, phthalic anhydride, benzoic anhydride, and 5-norbornene-2,3-dicarboxylic acid anhydride. Examples of the preferred carboxylic acid chloride compound include acetyl chloride, acrylic acid chloride, propionyl chloride, methacrylic acid chloride, pivaloyl chloride, cyclohexanecarbonyl chloride, 2-ethylhexanoyl chloride, cinnamoyl chloride, 1-adamantanecarbonyl chloride, heptafluorobutyryl chloride, stearic acid chloride, and benzoyl chloride.

In addition, by using a terminal blocking agent including a group having an ethylenically unsaturated bond, the structure represented by any of Formula (3-1) to Formula (3-3) can also be introduced into the specific resin.

For example, by using a monoamine represented by Formula (AM-1), it is possible to introduce a structure having a structure represented by Formula (3-1) at the terminal, in which R³¹ is a group represented by Formula (R3-1).

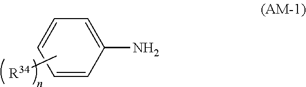

The reaction temperature in the reaction for obtaining the reaction product is preferably 150° C. to 350° C. and more preferably 160° C. to 250° C. in a case of performing thermal imidization.

However, as described above, in the present invention, it is also preferable to carry out the reaction at a low temperature by chemical imidization, and the reaction temperature in this case is preferably room temperature (23° C.) to 100° C., and more preferably 50° C. to 90° C.

The reaction temperature may be determined with reference to known conditions, by considering the rate of change in imide group value described later, or by similar methods.

The reaction time (that is, the time exposed to the above-described reaction temperature) in the reaction for obtaining the reaction product is not particularly limited, and may be determined with reference to known conditions or by considering the rate of change in imide group value described later, and the like, and it can be set to, for example, 30 minutes to 24 hours, and it is preferably 1 to 15 hours, more preferably 2 to 10 hours, and still more preferably 3 to 8 hours.

After the reaction to obtain the reaction product, a step of precipitating a solid may be included. Specifically, it is possible to obtain a specific resin such as a polyimide by filtering out a water-absorbing by-product of the dehydration condensing agent that is present together in the reaction solution as necessary, subsequently putting the obtained polymer component in a poor solvent such as water, an aliphatic lower alcohol, or a mixed solution thereof, precipitating the polymer component to be precipitated as a solid, and then carrying out drying. In order to improve the degree of purification, operations such as redissolution, reprecipitation, and drying of the specific resin such as the polyimide may be repeated. Further, a step of removing ionic impurities using an ion exchange resin may be included.

The specific resin is preferably a resin in which a rate of change in imide group value calculated by the following expression before and after heating at 350° C. and 1 atm for 1 hour is 25% or less.

$$Rate of change ( \% ) = ( Im 2 - Im 1 ) \times 10 \; 0 / Im 1$$

Im1: an imide group value before heating (mmol/g)
Im2: an imide group value (mmol/g) after heating at 350° C. and 1 atm for 1 hour

The above-described rate of change is preferably 15% or less and more preferably 10% or less. In addition, the lower limit of the above-described rate of change is not particularly limited, and may be 0% or more.

The above-described rate of change is measured, for example, by the following method.

The infrared absorption spectrum of the specific resin is measured, and a peak intensity P1 in the vicinity of 1,377 cm⁻¹, which is the absorption peak derived from the imide bond, is obtained. Next, the specific resin is subjected to a heat treatment at 350° C. and 1 atm for 1 hour, and then the infrared absorption spectrum is measured again to obtain a peak intensity P2 in the vicinity of 1,377 cm⁻¹. Using the obtained peak intensities P1 and P2, the rate of change in imide group value can be determined based on the following expression. The peak intensity P1 is an index indicating an imide group value Im1 before heating, and the peak intensity P2 is an index indicating an imide group value Im2 after heating.

$$Ra te of change ( \% ) in imide group value = ( peak intensity P 2 - peak intensity P 1 ) \times 100 / peak intensity P 1$$

The content of the repeating unit represented by Formula (1-1) is preferably 50% by mass or more, more preferably 70% by mass or more, still more preferably 80% by mass or more, and particularly preferably 90% by mass or more with respect to the total mass of the specific resin. The upper limit of the content is not particularly limited, and may be 100% by mass.

In addition, the specific resin may contain two or more kinds of repeating units represented by Formula (1-1). In that case, it is preferable that the total amount is within the above-described range.

The content of the repeating unit represented by Formula (1-2) with respect to the total mass of the specific resin is preferably 20% by mass or more, more preferably 30% by mass or more, and still more preferably 40% by mass or more. The upper limit of the content is not particularly limited, and may be 100% by mass.

In addition, the specific resin may contain two or more kinds of repeating units represented by Formula (1-2). In that case, it is preferable that the total amount is within the above-described range.

The content of the repeating unit represented by Formula (1-3) with respect to the total mass of the specific resin is preferably 20% by mass or more, more preferably 30% by mass or more, and still more preferably 40% by mass or more. The upper limit of the content is not particularly limited, and may be 100% by mass.

In addition, the specific resin may contain two or more kinds of repeating units represented by Formula (1-1). In that case, it is preferable that the total amount is within the above-described range.

The weight-average molecular weight (Mw) of the specific resin is preferably 3,000 to 100,000.

The lower limit of the above-described Mw is preferably 5,000 or more, more preferably 8,000 or more, and still more preferably 10,000 or more.

The upper limit of the Mw is preferably 50,000 or less, more preferably 40,000 or less, and still more preferably 25,000 or less.

In a case where the weight-average molecular weight is set to 3,000 or more, it is possible to improve the breakage resistance of the film after curing. In order to obtain an organic film having excellent mechanical properties (for example, breaking elongation), the weight-average molecular weight is particularly preferably 5,000 or more.

The number-average molecular weight (Mn) of the specific resin is preferably 1,000 to 40,000, more preferably 2,000 to 30,000, still more preferably 5,000 to 20,000, and particularly preferably 6,000 to 12,000.

The dispersity of the molecular weight of the specific resin is preferably 1.5 or more, more preferably 1.8 or more, and still more preferably 2.0 or more. In the present specification, the dispersity of the molecular weight is a value obtained by calculating “weight-average molecular weight/number-average molecular weight”. The upper limit value of the dispersity of the molecular weight of the specific resin is not particularly specified, but is, for example, preferably 7.0 or less, more preferably 6.5 or less, still more preferably 6.0 or less, even more preferably 4.5 or less, and particularly preferably 3.0 or less.

In a case where the resin composition contains a plurality of kinds of specific resins as the specific resin, it is preferable that the weight-average molecular weight, the number-average molecular weight, and the dispersity of at least one kind of specific resin are in the above ranges. It is also preferable that the weight-average molecular weight, the number-average molecular weight, and the dispersity, calculated by using the plurality of kinds of specific resins as one resin, are within the above ranges.

### Specific Example

Specific examples of the specific resin include polyimides (A-1) to (A-20) in Examples described later, but the present invention is not limited thereto.

### [Content]

The content of the specific resin in the resin composition according to the embodiment of the present invention is preferably 20% by mass or more, more preferably 30% by mass or more, still more preferably 40% by mass or more, even still more preferably 50% by mass or more, and most preferably 60% by mass or more, with respect to the total solid content of the resin composition. In addition, the content of the resin in the resin composition according to the embodiment of the present invention is preferably 99.5% by mass or less, more preferably 99% by mass or less, still more preferably 98% by mass or less, still more preferably 97% by mass or less, and even still more preferably 95% by mass or less, with respect to the total solid content of the resin composition.

The resin composition according to the embodiment of the present invention may contain only one kind of specific resin or may contain two or more kinds thereof. In a case where two or more kinds thereof are contained, the total amount thereof is preferably within the above range.

### <Another Resin>

The resin composition according to the embodiment of the present invention may contain the above-described specific resin and another resin (hereinafter, also simply referred to as “the other resin”) that is different from the specific resin.

Examples of the other resins include resins different from the specific resin and corresponding to polyimide precursors, polyimides, polybenzoxazole precursors, polybenzoxazole, polyamideimide precursors, polyamideimide, aromatic polyether, phenol resin, polyamide, epoxy resin, polysiloxane, resin including a siloxane structure, (meth)acrylic resin, (meth)acrylamide resin, urethane resin, butyral resin, styryl resin, polyether resin, and polyester resin.

Examples of the other polyimide precursors, the other polyimides, the polybenzoxazole precursors, the polybenzoxazoles, the polyamideimide precursors, and the polyamideimides include the compounds described in paragraphs 0017 to 0138 of WO2022/145355A. The above description is incorporated in the present specification.

The aromatic polyether is not particularly limited, but is preferably a polyphenylene ether.

The polyphenylene ether preferably includes a repeating unit represented by Formula (PE).

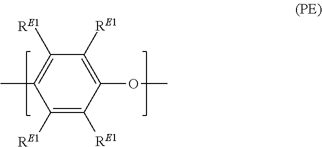

In Formula (PE), R^{E1} represents a hydrogen atom or a substituent. Examples of the substituent include a halogen atom, an alkyl group which may have a substituent, an alkoxy group which may have a substituent, an amino group which may have a substituent, a nitro group, and a carboxy group.

In addition, the polyphenylene ether is also preferably a compound having a polymerizable group.

As the polymerizable group, an epoxy group, an oxetanyl group, an oxazolyl group, a methylol group, an alkoxymethyl group, an acyloxymethyl group, a blocked isocyanate group, or a group having an ethylenically unsaturated bond is preferable, and a group having an ethylenically unsaturated bond is more preferable.

Examples of the group having an ethylenically unsaturated bond include a vinyl group, an allyl group, an isoallyl group, a 2-methylallyl group, a group (for example, a vinylphenyl group) having an aromatic ring that is directly bonded to a vinyl group, and a (meth)acrylamide group, a (meth)acryloyloxy group, where a vinylphenyl group, a (meth)acrylamide group, or a (meth)acryloyloxy group is preferable, a vinylphenyl group or a (meth)acryloyloxy group is more preferable, and a (meth)acryloyloxy group is still more preferable.

In a case where the polyphenylene ether is a compound having a polymerizable group, the position of the polymerizable group is not particularly limited, but for example, a structure in which the polymerizable group is introduced at the terminal of the main chain is preferable.

The polyphenylene ether may include other repeating units. However, the content of the other repeating units is preferably 30% by mass or less, more preferably 20% by mass or less, and still more preferably 10% by mass or less with respect to the total mass of the polyphenylene ether.

The number-average molecular weight of the polyphenylene ether is not particularly limited, but is preferably 500 to 50,000.

The lower limit of the number-average molecular weight is preferably 800 or higher, more preferably 1000 or higher, and still more preferably 1500 or higher.

The upper limit of the number-average molecular weight is preferably 30,000 or less, more preferably 20,000 or less, and still more preferably 10,000 or less.

Specific examples of the polyphenylene ether (PPE) include poly(2,6-dimethyl-1,4-phenylene ether), poly(2-methyl-6-ethyl-1,4-phenylene ether), poly(2-methyl-6-phenyl-1,4-phenylene ether), poly(2,6-dichloro-1,4-phenylene ether), a copolymer of 2,6-dimethylphenol and other phenols (for example, 2,3,6-trimethylphenol, 2-methyl-6-butylphenol, and the like), a polyphenylene ether copolymer obtained by coupling 2,6-dimethylphenol with biphenols or bisphenols, and a polyphenylene ether having a linear structure or a branched structure, which is obtained by heating poly(2,6-dimethyl-1,4-phenylene ether) and the like with phenolic compounds such as bisphenols or trisphenols in a toluene solvent in the presence of an organic peroxide and performing a redistribution reaction, but the present invention is not limited thereto.

In a case where the resin composition according to the embodiment of the present invention contains the other resin, the content of the other resin is preferably 0.01% by mass or more, more preferably 0.05% by mass or more, still more preferably 1% by mass or more, even still more preferably 2% by mass or more, even still more preferably 5% by mass or more, and even further still more preferably 10% by mass or more, with respect to the total solid content of the resin composition.

The content of the other resin in the resin composition according to the embodiment of the present invention is preferably 80% by mass or less, more preferably 75% by mass or less, still more preferably 70% by mass or less, even still more preferably 60% by mass or less, and even further still more preferably 50% by mass or less, with respect to the total solid content of the resin composition.

As one preferred aspect of the resin composition according to the embodiment of the present invention, an aspect in which the content of the other resin is a low content can be adopted. In the above aspect, the content of the other resin is preferably 20% by mass or less, more preferably 15% by mass or less, still more preferably 10% by mass or less, even still more preferably 5% by mass or less, and even further still more preferably 1% by mass or less, with respect to the total solid content of the resin composition. The lower limit of the content is not particularly limited, and it may be any content of 0% by mass or more.

In addition, in a case where the resin composition according to the embodiment of the present invention contains another resin, the content of the specific resin with respect to the total content of the specific resin and the other resin is preferably 40% to 90% by mass, more preferably 50% to 80% by mass, and still more preferably 55% to 70% by mass.

The resin composition according to the embodiment of the present invention may contain only one kind of the other resin or may contain two or more kinds thereof. In a case where two or more kinds thereof are contained, the total amount thereof is preferably within the above range.

### <Polymerizable Compound Having Ethylenically Unsaturated Bond>

The resin composition according to the embodiment of the present invention contains a polymerizable compound having an ethylenically unsaturated bond.

The polymerizable compound having an ethylenically unsaturated bond has a group including an ethylenically unsaturated bond. Examples of the group containing an ethylenically unsaturated bond include a vinyl group, an allyl group, a vinylphenyl group, a (meth)acryloyl group, a maleimide group, and a (meth)acrylamide group.

Among these, a (meth)acryloyl group, a (meth)acrylamide group, or a vinylphenyl group is preferable, and from the viewpoint of reactivity, a (meth)acryloyl group is more preferable.

The polymerizable compound having an ethylenically unsaturated bond is preferably a compound having one or more groups each having an ethylenically unsaturated bond, but more preferably a compound having two or more groups each having an ethylenically unsaturated bond. The polymerizable compound having an ethylenically unsaturated bond may have three or more groups each of which has an ethylenically unsaturated bond.

The compound having two or more groups each having an ethylenically unsaturated bond is preferably a compound having 2 to 15 groups each having an ethylenically unsaturated bond, more preferably a compound having 2 to 10 groups each having an ethylenically unsaturated bond, and still more preferably a compound having 2 to 6 groups each having an ethylenically unsaturated bond.

From the viewpoint of the film hardness of the pattern (cured substance) to be obtained, the resin composition according to the embodiment of the present invention preferably contains a compound having two groups each containing ethylenically unsaturated bonds and a compound having three or more groups each containing ethylenically unsaturated bonds.

The molecular weight of the polymerizable compound having an ethylenically unsaturated bond is preferably 2,000 or less, more preferably 1,500 or less, and still more preferably 900 or less. The lower limit of the molecular weight of the polymerizable compound having an ethylenically unsaturated bond is preferably 100 or more.

Specific examples of the polymerizable compound having an ethylenically unsaturated bond include unsaturated carboxylic acids (for example, acrylic acid, methacrylic acid, itaconic acid, crotonic acid, isocrotonic acid, and maleic acid), and esters and amides thereof, and esters formed from unsaturated carboxylic acids and polyhydric alcohol compounds or amides formed from unsaturated carboxylic acids and polyvalent amine compounds are preferable. In addition, addition reaction products produced by reacting unsaturated carboxylic acid esters or amides, having a nucleophilic substituent such as a hydroxy group, an amino group, or a sulfanyl group, with monofunctional or polyfunctional isocyanates or epoxies, dehydration condensation reaction products produced by reacting the above esters or amides with a monofunctional or polyfunctional carboxylic acid, or the like are also suitably used. In addition, an addition reaction product produced by reacting unsaturated carboxylic acid esters or amides, having an electrophilic substituent such as an isocyanate group or an epoxy group, with monofunctional or polyfunctional alcohols, amines, or thiols, and further, a substitution reaction product produced by reacting unsaturated carboxylic acid esters or amides, having an eliminable substituent such as a halogeno group or a tosyloxy group, with monofunctional or polyfunctional alcohols, amines, or thiols is also suitable. In addition, as other examples, it is also possible to use a group of compounds in which the unsaturated carboxylic acid described above is replaced with an unsaturated phosphonic acid, a vinylbenzene derivative such as styrene, a vinyl ether, an allyl ether, or the like. Regarding the specific examples thereof, reference can be made to the description of paragraphs 0113 to 0122 of JP2016-027357A, the content of which is incorporated in the present specification.

The polymerizable compound having an ethylenically unsaturated bond is also preferably a compound having a boiling point of 100° C. or higher under normal pressure. Examples of the compound having a boiling point of 100° C. or higher under normal pressure include the compounds described in paragraph 0203 of WO2021/112189A. The content thereof is incorporated in the present specification.

Preferred examples of the polymerizable compound having an ethylenically unsaturated bond other than those described above include the radically polymerizable compounds described in paragraphs 0204 to 0208 of WO2021/112189A. The content thereof is incorporated in the present specification.

As the polymerizable compound having an ethylenically unsaturated bond, dipentaerythritol triacrylate (as a commercially available product, KAYARAD D-330 (manufactured by Nippon Kayaku Co., Ltd.), dipentaerythritol tetraacrylate (as a commercially available product, KAYARAD D-320 (manufactured by Nippon Kayaku Co., Ltd.), A-TMMT (manufactured by Shin-Nakamura Chemical Co., Ltd.), dipentaerythritol penta(meth)acrylate (as a commercially available product, KAYARAD D-310 (manufactured by Nippon Kayaku Co., Ltd.), dipentaerythritol hexa(meth)acrylate (as a commercially available product, KAYARAD DPHA (manufactured by Nippon Kayaku Co., Ltd.), A-DPH (manufactured by Shin-Nakamura Chemical Co., Ltd.), or a structure in which a (meth)acryloyl group thereof is bonded through an ethylene glycol residue or a propylene glycol residue is preferable. Oligomer types thereof can also be used.

Examples of the commercially available product of the polymerizable compound having an ethylenically unsaturated bond include SR-494 which is a tetrafunctional acrylate having four ethyleneoxy chains, SR-209, 231, and 239 which are a difunctional methacrylate having four ethyleneoxy chains (all of which are manufactured by Sartomer Company Inc.), DPCA-60 which is a hexafunctional acrylate having six pentyleneoxy chains and TPA-330 which is a trifunctional acrylate having three isobutylene oxy chains (all of which are manufactured by Nippon Kayaku Co., Ltd.), UAS-10 and UAB-140 which are a urethane oligomer (all of which are manufactured by Nippon Paper Industries Co., Ltd.), NK Ester M-40G, NK Ester 4G, NK Ester M-9300, NK Ester A-9300, and UA-7200 (all of which are manufactured by Shin-Nakamura Chemical Co., Ltd.), DPHA-40H (manufactured by Nippon Kayaku Co., Ltd.), UA-306H, UA-306T, UA-306I, AH-600, T-600, and AI-600 (all of which are manufactured by KYOEISHA CHEMICAL Co., Ltd.), and Brenmer PME400 (manufactured by NOF Corporation).

As the polymerizable compound having an ethylenically unsaturated bond, urethane acrylates described in JP1973-041708B (JP-S48-041708B), JP1976-037193A (JP-S51-037193A), JP1990-032293B (JP-H02-032293B), and JP1990-016765B (JP-H02-016765B), or urethane compounds having an ethylene oxide-based skeleton, described in JP1983-049860B (JP-S58-049860B), JP1981-017654B (JP-S56-017654B), JP1987-039417B (JP-S62-039417B), and JP1987-039418B (JP-S62-039418B), are also suitable. As the polymerizable compound having an ethylenically unsaturated bond, the compounds having an amino structure or a sulfide structure in a molecule as described in JP1988-277653A (JP-S63-277653A), JP1988-260909A (JP-S63-260909A), and JP1989-105238A (JP-H01-105238A) can also be used.

The polymerizable compound having an ethylenically unsaturated bond may be a compound having an acid group such as a carboxy group or a phosphoric acid group. The polymerizable compound having an ethylenically unsaturated bond and an acid group is preferably an ester of an aliphatic polyhydroxy compound and an unsaturated carboxylic acid and more preferably a polymerizable compound having an ethylenically unsaturated bond obtained by reacting an unreacted hydroxy group of an aliphatic polyhydroxy compound with a non-aromatic carboxylic acid anhydride to have an acid group. The polymerizable compound having an ethylenically unsaturated bond is particularly preferably a compound in which an aliphatic polyhydroxy compound is pentaerythritol or dipentaerythritol in a polymerizable compound having an ethylenically unsaturated bond and an acid group obtained by reacting an unreacted hydroxy group of the aliphatic polyhydroxy compound with a non-aromatic carboxylic acid anhydride. Examples of the commercially available product thereof include M-510 and M-520 as polybasic acid-modified acrylic oligomers which are manufactured by Toagosei Co., Ltd.

The acid value of the polymerizable compound having an ethylenically unsaturated bond and an acid group is preferably 0.1 to 300 mgKOH/g and more preferably 1 to 100 mgKOH/g. In a case where the acid value of the polymerizable compound having an ethylenically unsaturated bond is within the above-described range, excellent production handling properties are exhibited, and excellent developability is exhibited. In addition, good polymerization properties are exhibited. The acid value is measured in accordance with the description of JIS K 0070: 1992.

As the polymerizable compound having an ethylenically unsaturated bond, a polymerizable compound having an ethylenically unsaturated bond and having at least one selected from the group consisting of a urea bond and a urethane bond (hereinafter, also referred to as a “polymerizable compound U”) is also preferable.

In the present invention, the urea bond is a bond represented by *—NR^{N}—C(═O)—NR^{N}*, where R^{N}'s each independently represent a hydrogen atom or a monovalent organic group, and *'s each represent a bonding site to a carbon atom.

In the present invention, the urethane bond is a bond represented by *—O—C(═O)—NR^{N}—*, where R^{N} represents a hydrogen atom or a monovalent organic group, and *'s each represent a bonding site to a carbon atom.

In a case where the resin composition contains a polymerizable compound U, chemical resistance, resolution, and the like may be improved.

The mechanism by which the above-described effect is obtained is not revealed; however, it is considered that, for example, a part of the polymerizable compound U undergoes thermal decomposition during curing by heating or the like to generate an amine or the like, and the amine or the like accelerates the cyclization of the precursor of the cyclization resin such as the polyimide precursor.

The polymerizable compound U may have only one urea bond or urethane bond, may have one or more urea bonds and one or more urethane bonds, may have two or more urea bonds without having a urethane bond, or may have two or more urethane bonds without having a urea bond.

The total number of urea bonds and urethane bonds in the polymerizable compound U is 1 or more, and it is preferably 1 to 10, more preferably 1 to 4, and still more preferably 1 or 2.

In a case where the polymerizable compound U does not have a urethane bond, the number of urea bonds in the polymerizable compound U is 1 or more, and it is preferably 1 to 10, more preferably 1 to 4, and still more preferably 1 or 2.

In a case where the polymerizable compound U does not have a urea bond, the number of urethane bonds in the polymerizable compound U is 1 or more, and it is preferably 1 to 10, more preferably 1 to 4, and still more preferably 1 or 2.

The radically polymerizable group in the polymerizable compound U is not particularly limited; however, examples thereof include a vinyl group, an allyl group, a (meth)acryloyl group, a (meth)acryloxy group, a (meth)acrylamide group, a vinylphenyl group, and a maleimide group, where a (meth)acryloxy group, a (meth)acrylamide group, a vinylphenyl group, or a maleimide group is preferable, and a (meth)acryloxy group is more preferable.

In a case where the polymerizable compound U has two or more radically polymerizable groups, the structures of the respective radically polymerizable groups may be the same or different from each other.

The number of radically polymerizable groups in the polymerizable compound U may be only one or may be two or more, and it is preferably 1 to 10, more preferably 1 to 6, and particularly preferably 1 to 4.

The radically polymerizable group value (the mass of the compound per 1 mol of the radically polymerizable group) in the polymerizable compound U is preferably 150 to 400 g/mol.

From the viewpoint of the chemical resistance of the cured substance, the lower limit of the above-described radically polymerizable group value is more preferably 200 g/mol or more, still more preferably 210 g/mol or more, even more preferably 220 g/mol or more, even still more preferably 230 g/mol or more, even still more preferably 240 g/mol or more, and particularly preferably 250 g/mol or more.

From the viewpoint of developability, the upper limit of the above-described radically polymerizable group value is more preferably 350 g/mol or less, still more preferably 330 g/mol or less, and particularly preferably 300 g/mol or less.

Among the above, the polymerizable group value of the polymerizable compound U is preferably 210 to 400 g/mol and more preferably 220 to 400 g/mol.

The polymerizable compound U has preferably, for example, a structure represented by Formula (U-1).

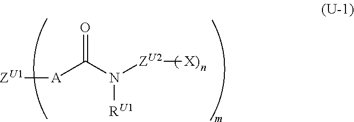

In Formula (U-1), R^{U1} is a hydrogen atom or a monovalent organic group, A is —O—, or —NR^{N}—, R^{N} is a hydrogen atom or a monovalent organic group, Z^{U1} is an m-valent organic group, Z^{U2} is an (n+1)-valent organic group, X is a radically polymerizable group, n is an integer of 1 or more, and m is an integer of 1 or more.

R^{U1} is preferably a hydrogen atom, an alkyl group, or an aromatic hydrocarbon group, and more preferably a hydrogen atom.

R^{N} is preferably a hydrogen atom, an alkyl group, or an aromatic hydrocarbon group, and more preferably a hydrogen atom.

Z^{U1} is preferably a hydrocarbon group, —O—, —C(═O)—, —S—, —S(═O)₂—, —NR^{N}—, or a group in which two or more of these are bonded, and more preferably a hydrocarbon group or a group in which a hydrocarbon group is bonded to at least one group selected from the group consisting of —O—, —C(═O)—, —S—, —S(═O)₂—, and —NR^{N}—.

As the above-described hydrocarbon group, a hydrocarbon group having 20 or less carbon atoms is preferable, a hydrocarbon group having 18 or less carbon atoms is more preferable, and a hydrocarbon group having 16 or less carbon atoms is still more preferable. Examples of the above-described hydrocarbon group include a saturated aliphatic hydrocarbon group, an aromatic hydrocarbon group, and a group represented by bonding of these groups. R^{N} represents a hydrogen atom or a monovalent organic group, and it is preferably a hydrogen atom or a hydrocarbon group, more preferably a hydrogen atom or an alkyl group, and still more preferably a hydrogen atom or a methyl group.

Z^{U2} is preferably a hydrocarbon group, —O—, —C(═O)—, —S—, —S(═O)₂—, —NR^{N}—, or a group in which two or more of these are bonded, and more preferably a hydrocarbon group or a group in which a hydrocarbon group is bonded to at least one group selected from the group consisting of —O—, —C(═O)—, —S—, —S(═O)₂—, and —NR^{N}—.

Examples of the hydrocarbon group include the same ones as those exemplified as Z^{U1}, and the same applies to the preferred aspect thereof.

X is not particularly limited; however, examples thereof include a vinyl group, an allyl group, a (meth)acryloyl group, a (meth)acryloxy group, a (meth)acrylamide group, a vinylphenyl group, and a maleimide group, where a (meth)acryloxy group, a (meth)acrylamide group, a vinylphenyl group, or a maleimide group is preferable, and a (meth)acryloxy group is more preferable.

n is preferably an integer of 1 to 10, more preferably an integer of 1 to 4, still more preferably 1 or 2, and particularly preferably 1.

m is preferably an integer of 1 to 10, more preferably an integer of 1 to 4, and still more preferably 1 or 2.

It is also preferable that the polymerizable compound U has at least one of a hydroxy group, an alkyleneoxy group, an amide group, or a cyano group.

From the viewpoint of the chemical resistance of the cured film to be obtained, the hydroxy group may be an alcoholic hydroxy group or a phenolic hydroxy group; however, it is preferably an alcoholic hydroxy group.

From the viewpoint of the chemical resistance of the cured film to be obtained, the alkyleneoxy group is preferably an alkyleneoxy group having 2 to 20 carbon atoms, more preferably an alkyleneoxy group having 2 to 10 carbon atoms, still more preferably an alkyleneoxy group having 2 to 4 carbon atoms, even still more preferably an ethylene group or a propylene group, and particularly preferably an ethylene group.

The alkyleneoxy group may be contained in the polymerizable compound U as a polyalkyleneoxy group. The number of repetitions of the alkyleneoxy group in this case is preferably 2 to 10 and more preferably 2 to 6.

The amide group refers to a bond represented by —C(═O)—NR^{N}—. R^{N} is as described above. In a case where the polymerizable compound U has an amide group, the polymerizable compound U can include, for example, an amide group as a group represented by R—C(═O)—NR^{N}—* or a group represented by *—C(═O)NR^{N}—. R^{N} represents a hydrogen atom or a monovalent substituent, and it is preferably a hydrogen atom or a hydrocarbon group and more preferably a hydrogen atom, an alkyl group, or an aromatic hydrocarbon group.

The polymerizable compound U may have, in the molecule, two or more structures selected from the group consisting of a hydroxy group, an alkyleneoxy group (however, a polyalkyleneoxy group in a case of constituting a polyalkyleneoxy group), an amide group, and a cyano group; however, an aspect in which only one structure is contained in the molecule is also preferable.

The hydroxy group, the alkyleneoxy group, the amide group, and the cyano group may be present at any position of the polymerizable compound U. However, from the viewpoint of chemical resistance, it is also preferable that the polymerizable compound U is such that at least one selected from the group consisting of the hydroxy group, the alkyleneoxy group, the amide group, and the cyano group, and at least one radically polymerizable group contained in the polymerizable compound U are linked by a linking group (hereinafter, also referred to as a “linking group L2-1”) containing a urea bond or a urethane bond.

In particular, in a case where the polymerizable compound U contains only one radically polymerizable group, it is preferable that the radically polymerizable group contained in the polymerizable compound U and at least one selected from the group consisting of a hydroxy group, an alkyleneoxy group, an amide group, and a cyano group are linked by a linking group including a urea bond or a urethane bond (hereinafter, also referred to as a “linking group L2-2”).

In a case where the polymerizable compound U contains an alkyleneoxy group (however, a polyalkyleneoxy group in a case of constituting a polyalkyleneoxy group) and has the above-described linking group L2-1 or the above-described linking group L2-2, a structure that is bonded to a side of the alkyleneoxy group (however, a polyalkyleneoxy group in a case of constituting a polyalkyleneoxy group) opposite to the linking group L2-1 or the linking group L2-2 is not particularly limited; however, it is preferably a hydrocarbon group, a radically polymerizable group, or a group represented by a combination thereof. As the above-described hydrocarbon group, a hydrocarbon group having 20 or less carbon atoms is preferable, a hydrocarbon group having 18 or less carbon atoms is more preferable, and a hydrocarbon group having 16 or less carbon atoms is still more preferable. Examples of the above-described hydrocarbon group include a saturated aliphatic hydrocarbon group, an aromatic hydrocarbon group, and a group represented by bonding of these groups. In addition, the preferred aspect of the radically polymerizable group is the same as the preferred aspect of the radically polymerizable group in the above-described polymerizable compound U.

In a case where the polymerizable compound U contains an amide group and has the linking group L2-1 or the linking group L2-2, a structure that is bonded to a side of the amide group opposite to the linking group L2-1 or the linking group L2-2 is not particularly limited; however, it is preferably a hydrocarbon group, a radically polymerizable group, or a group represented by a combination thereof. As the above-described hydrocarbon group, a hydrocarbon group having 20 or less carbon atoms is preferable, a hydrocarbon group having 18 or less carbon atoms is more preferable, and a hydrocarbon group having 16 or less carbon atoms is still more preferable. In addition, examples of the above-described hydrocarbon group include a saturated aliphatic hydrocarbon group, an aromatic hydrocarbon group, and a group represented by bonding of these groups. The preferred aspect of the radically polymerizable group is the same as the preferred aspect of the radically polymerizable group in the above-described polymerizable compound U. In addition, in the above aspect, a carbon atom side of the amide group may be bonded to the linking group L2-1 or the linking group L2-2, or a nitrogen atom side of the amide group may be bonded to the linking group L2-1 or the linking group L2-2.

Among these, from the viewpoint of the adhesiveness to the base material, the chemical resistance, and the Cu void suppression, the polymerizable compound U preferably has a hydroxy group.

From the viewpoint of the compatibility with the specific resin and the like, the polymerizable compound U preferably contains an aromatic group.

The aromatic group is preferably directly bonded to a urea bond or a urethane bond contained in the polymerizable compound U. In a case where the polymerizable compound U contains two or more urea bonds or two or more urethane bonds, it is preferable that one of the urea bonds or urethane bonds is directly bonded to the aromatic group.

The aromatic group may be an aromatic hydrocarbon group or an aromatic heterocyclic group and may have a structure in which these groups form a fused ring; however, an aromatic hydrocarbon group is preferable.

The aromatic hydrocarbon group is preferably an aromatic hydrocarbon group having 6 to 30 carbon atoms, more preferably an aromatic hydrocarbon group having 6 to 20 carbon atoms, and still more preferably a group obtained by removing two or more hydrogen atoms from a benzene ring structure.

The aromatic heterocyclic group is preferably a 5-membered or 6-membered aromatic heterocyclic group. Examples of the aromatic heterocyclic ring in such an aromatic heterocyclic group include pyrrole, imidazole, triazole, tetrazole, pyrazole, furan, thiophene, oxazole, isoxazole, thiazole, pyridine, pyrazine, pyrimidine, pyridazine, and triazine. These rings may be further fused with another ring, for example, as in the case of indole or benzimidazole.

The heteroatom contained in the aromatic heterocyclic group is preferably a nitrogen atom, an oxygen atom, or a sulfur atom.

It is preferable that the aromatic group is included in, for example, a linking group that links two or more radically polymerizable groups and links a linking group having a urea bond or a urethane bond, or at least one selected from the group consisting of a hydroxy group, an alkyleneoxy group, an amide group, and a cyano group, to at least one radically polymerizable group contained in the polymerizable compound U.

The number of atoms (linking chain length) between the urea bond or urethane bond and the radically polymerizable group in the polymerizable compound U is not particularly limited; however, it is preferably 30 or less, more preferably 2 to 20, and still more preferably 2 to 10.

In a case where the polymerizable compound U contains two or more urea bonds or urethane bonds in total, the minimum number of atoms (linking chain length) between the urea bond or urethane bond and the radically polymerizable group is sufficient to be within the above-described range in a case where the polymerizable compound U contains two or more radically polymerizable groups, or in a case where the polymerizable compound U contains two or more urea bonds or two or more urethane bonds and two or more radically polymerizable groups.

In the present specification, the phrase “number of atoms (linking chain length) between the urea bond or urethane bond and the polymerizable group” refers to the smallest number of atoms (minimum number of atoms) for linking targets among the number of atoms for atomic chains on a path that links two atoms or atomic groups between the linking targets. For example, in a structure represented by the following Formula, the number of atoms (linking chain length) between the urea bond and the radically polymerizable group (methacryloyloxy group) is 2.

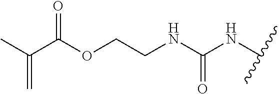 [SMILES: *NC(=O)NCCOC(=O)C(=C)C]

### [Symmetry Axis]

It is also preferable that the polymerizable compound U is a compound having a structure which does not have a symmetry axis.

The fact that the polymerizable compound U does not have a symmetry axis refers to that the polymerizable compound U is a left-right asymmetric compound which does not have an axis that generates the same molecule as the original molecule in a case where the entire compound is rotated. In addition, in a case where the structural formula of the polymerizable compound U is shown on the paper surface, the fact that the polymerizable compound U does not have a symmetry axis refers to that the structural formula of the polymerizable compound U cannot be shown in a form having a symmetry axis.

It is considered that the aggregation of the polymerizable compounds U is suppressed in the composition film in a case where the polymerizable compound U does not have a symmetry axis.

### [Molecular Weight]

The molecular weight of the polymerizable compound U is preferably 100 to 2,000, preferably 150 to 1,500, and more preferably 200 to 900.

A manufacturing method for the polymerizable compound U is not particularly limited; however, the polymerizable compound U can be obtained, for example, by reacting a radically polymerizable compound with a compound having an isocyanate group and a compound having at least one of a hydroxy group or an amino group.

Specific examples of the polymerizable compound U are shown below, but the polymerizable compound U is not limited thereto.

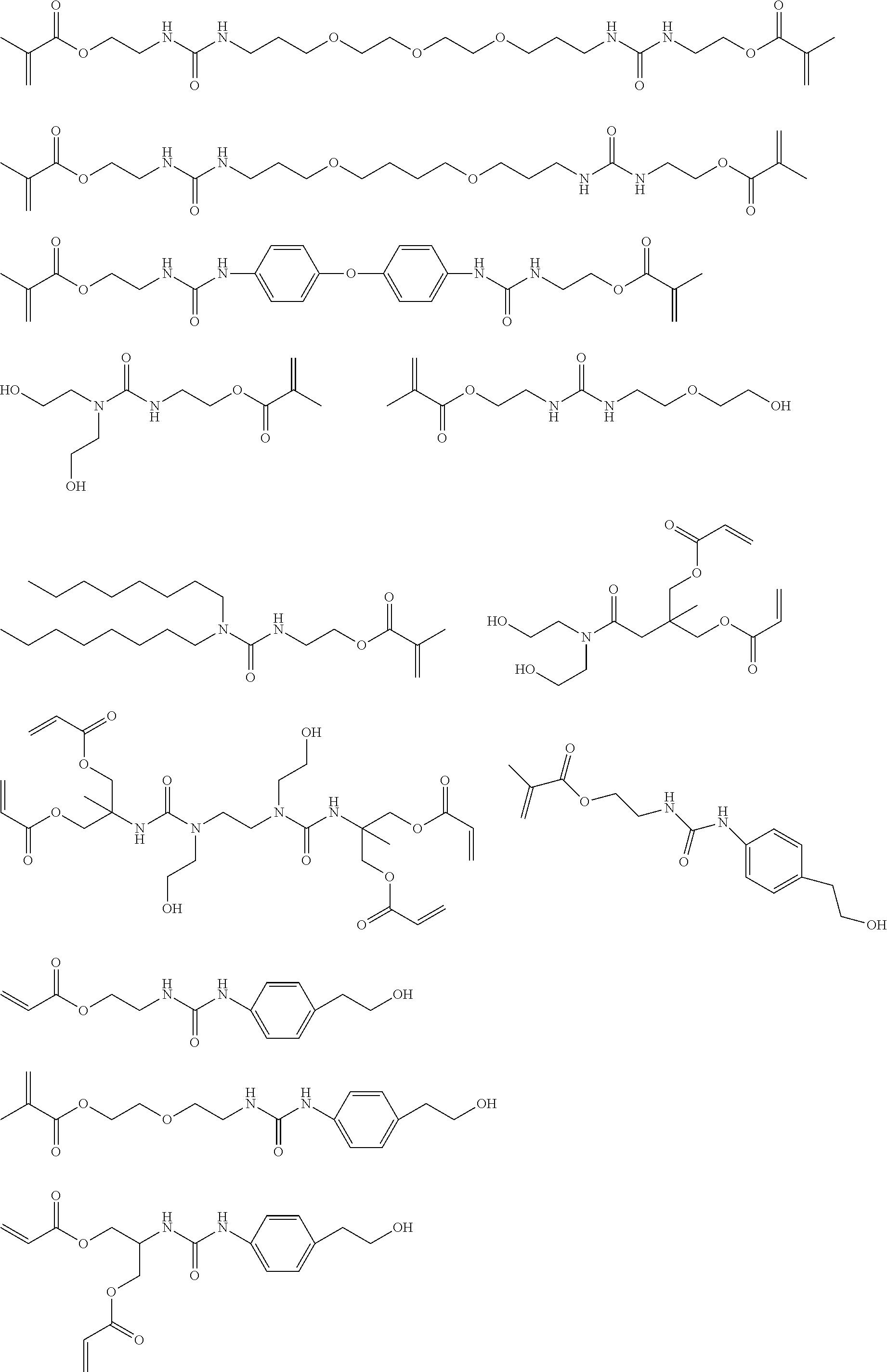

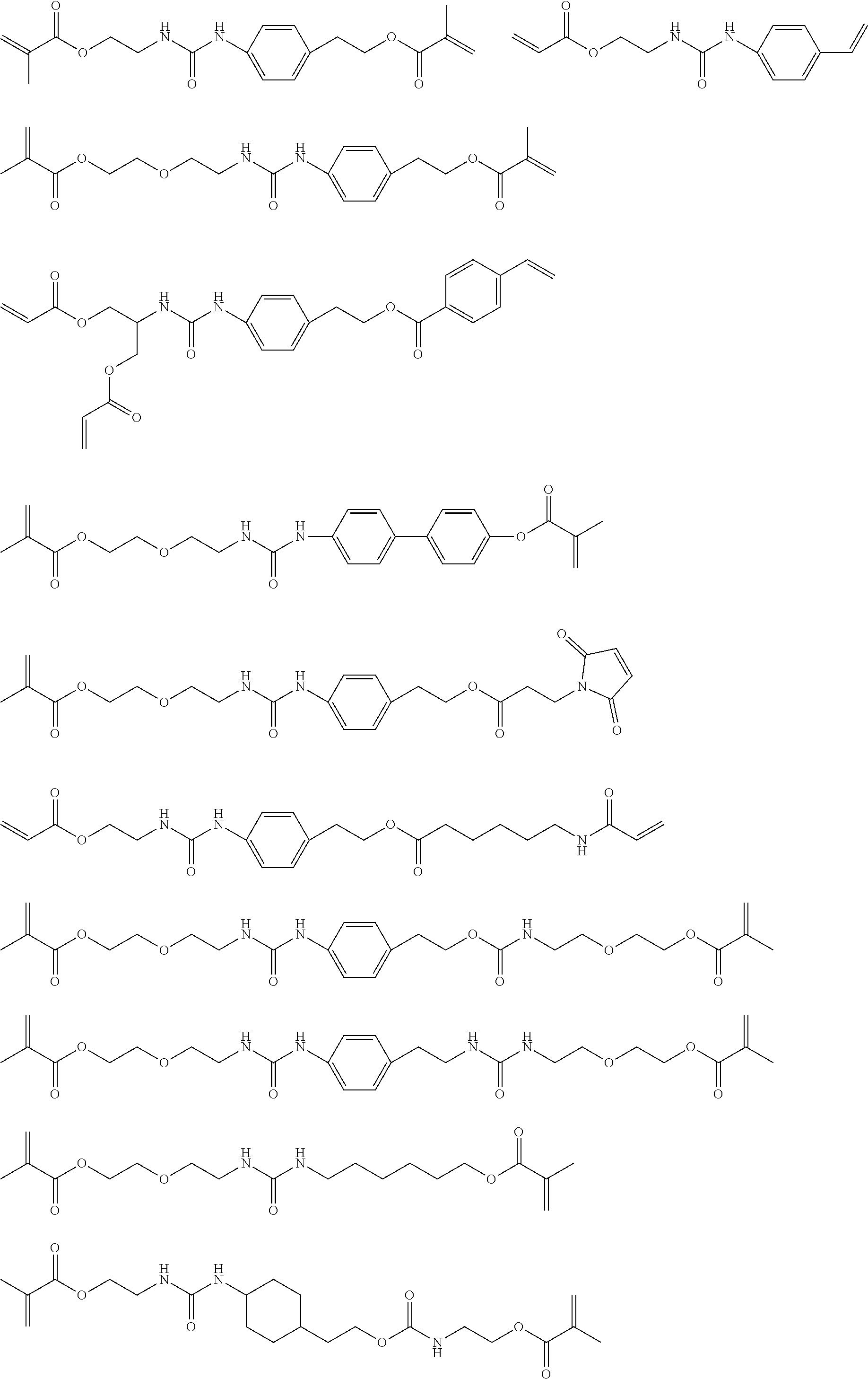

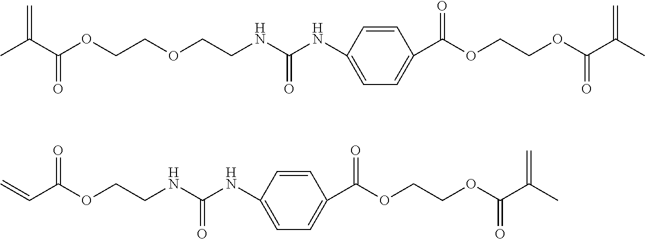

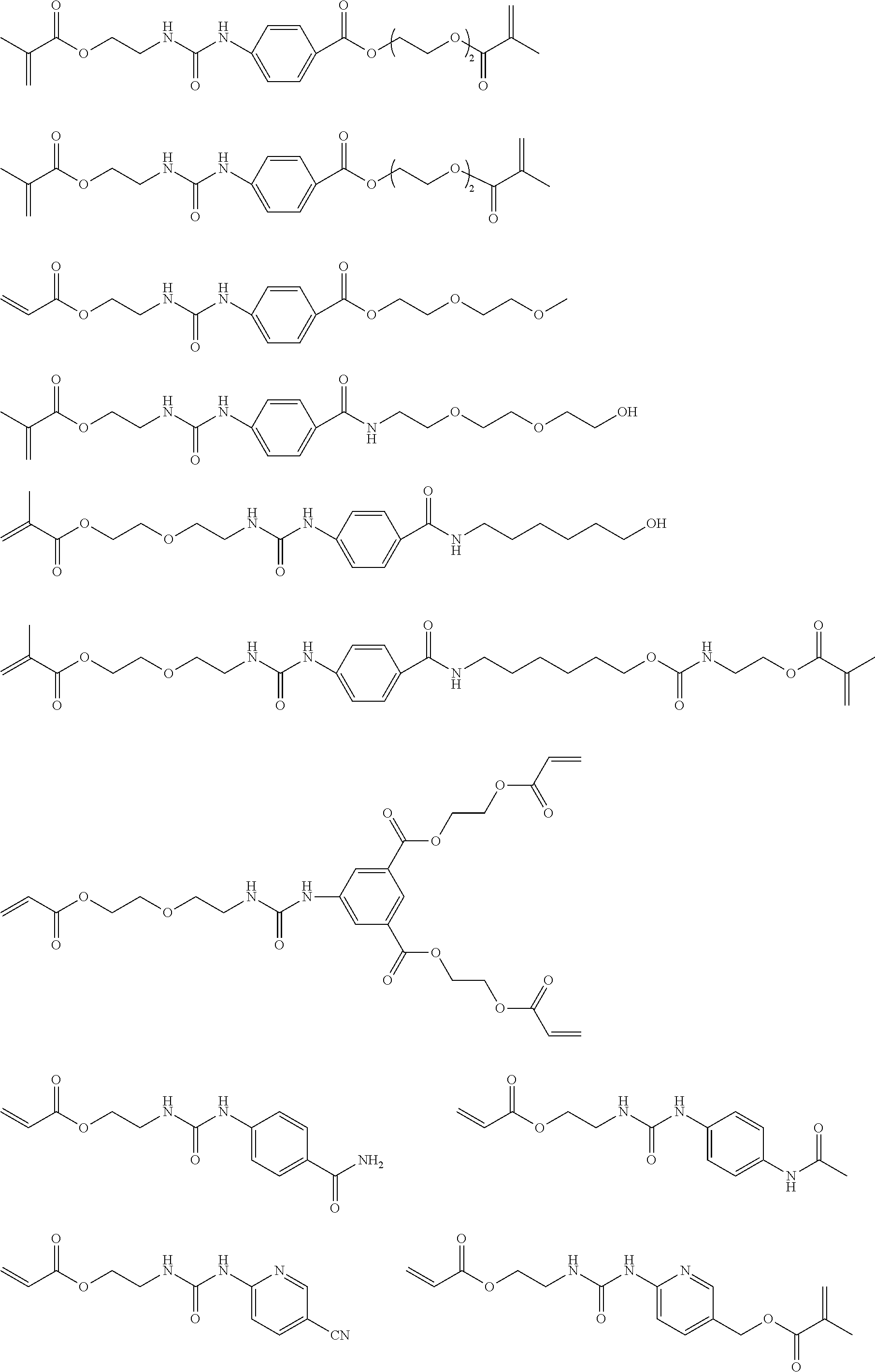

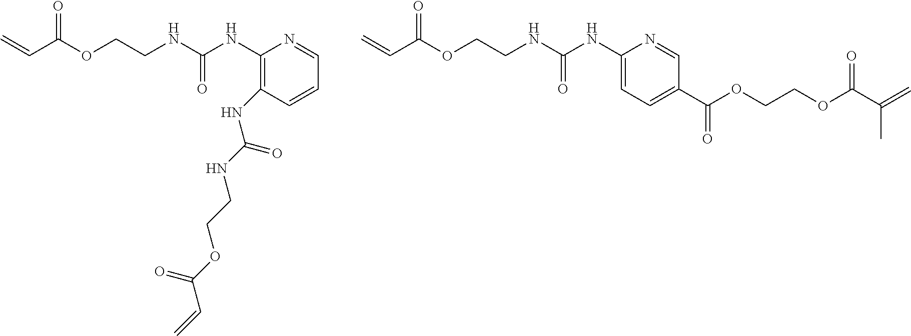

In the resin composition, it is preferable to use difunctional methacrylate or acrylate from the viewpoint of pattern resolution and film elasticity.

As the specific compound, the following compound can be used; triethylene glycol diacrylate, triethylene glycol dimethacrylate, tetraethylene glycol dimethacrylate, tetraethylene glycol diacrylate, polyethylene glycol (PEG) 200 diacrylate, PEG 200 dimethacrylate, PEG 600 diacrylate, PEG 600 dimethacrylate, polytetraethylene glycol diacrylate, polytetraethylene glycol dimethacrylate, dipropylene glycol diacrylate, tripropylene glycol diacrylate, neopentyl glycol diacrylate, neopentyl glycol dimethacrylate, 3-methyl-1,5-pentanediol diacrylate, 1,6-hexanediol diacrylate, 1,6-hexanediol dimethacrylate, dimethylol-tricyclodecanediacrylate, dimethylol-tricyclodecanedimethacrylate, a diacrylate of an ethylene oxide (EO) adduct of bisphenol A, a dimethacrylate of an EO adduct of bisphenol A, a diacrylate of a propylene oxide (PO) adduct of bisphenol A, a dimethacrylate of a PO adduct of bisphenol A, 2-hydroxy-3-acryloyloxypropyl methacrylate, isocyanuric acid EO-modified diacrylate, isocyanuric acid EO-modified dimethacrylate, another difunctional acrylate having a urethane bond, and difunctional methacrylate having a urethane bond. As necessary, two or more of these can be mixedly used.

It is noted that, for example, the PEG 200 diacrylate refers to a polyethylene glycol diacrylate having a polyethylene glycol chain Formula weight of about 200.

In the resin composition according to the embodiment of the present invention, from the viewpoint of suppressing warping of the pattern (cured substance), a polymerizable compound having only one ethylenically unsaturated bond (monofunctional polymerizable compound) can be preferably used as the polymerizable compound having an ethylenically unsaturated bond. As the monofunctional polymerizable compound, (meth)acrylic acid derivatives such as n-butyl (meth)acrylate, 2-ethylhexyl (meth)acrylate, 2-hydroxyethyl (meth)acrylate, butoxyethyl (meth)acrylate, carbitol (meth)acrylate, cyclohexyl (meth)acrylate, benzyl (meth)acrylate, phenoxyethyl (meth)acrylate, N-methylol (meth)acrylamide, glycidyl (meth)acrylate, polyethylene glycol mono(meth)acrylate, and polypropylene glycol mono(meth)acrylate, N-vinyl compounds such as N-vinylpyrrolidone and N-vinylcaprolactam, allyl glycidyl ethers, and the like are preferably used. As the monofunctional polymerizable compound, a compound having a boiling point of 100° C. or higher under normal pressure is also preferable in order to suppress volatilization before exposure.

In addition, examples of the polymerizable compound having two or more ethylenically unsaturated bonds include allyl compounds such as diallyl phthalate and triallyl trimellitate.

The content of the polymerizable compound having an ethylenically unsaturated bond is preferably more than 0% by mass and 60% by mass or less with respect to the total solid content of the resin composition. The lower limit thereof is more preferably 5% by mass or more. The upper limit thereof is more preferably 50% by mass or less and still more preferably 30% by mass or less.

The polymerizable compound having an ethylenically unsaturated bond may be used alone or in combination of two or more kinds thereof. In a case where two or more kinds thereof are used in combination, the total amount thereof is preferably in the above range.

### [Other Polymerizable Compounds]

It is also preferable that the resin composition according to the embodiment of the present invention further contains another polymerizable compound different from the above-described polymerizable compound having an ethylenically unsaturated bond.

The other polymerizable compound refers to a polymerizable compound other than the above-described polymerizable compound having an ethylenically unsaturated bond, where it is preferable that the compound is a compound having a plurality of groups, in the molecule, which promotes a reaction of forming a covalent bond between other compounds in the composition or reaction products thereof, by the photosensitization of the above-described photoacid generator photobase generator, and it is more preferable that the compound is a compound having a plurality of groups, in the molecule, which accelerates a reaction of forming a covalent bond between other compounds in the composition or reaction products thereof, by the action of the acid or the base.

Examples of the other polymerizable compounds include the compounds described in paragraphs 0179 to 0207 of WO2022/145355A. The above description is incorporated in the present specification.

### [Polymerization Initiator]

The resin composition according to the embodiment of the present invention preferably includes a polymerization initiator. The polymerization initiator may be a thermal polymerization initiator or a photopolymerization initiator; however, it is particularly preferable to contain a photopolymerization initiator.

The photopolymerization initiator is preferably a photoradical polymerization initiator. The photoradical polymerization initiator is not particularly limited and can be appropriately selected from publicly known photoradical polymerization initiators. For example, a photoradical polymerization initiator having photosensitivity to rays ranging from the ultraviolet ray range to the visible light range is preferable. In addition, it may be an activator that acts with a sensitizing agent to generate an active radical.

The photoradical polymerization initiator preferably contains at least one compound having a molar absorption coefficient of at least about 50 L·mol⁻¹·cm⁻¹ within a range of a wavelength of about 240 to 800 nm (preferably 330 to 500 nm). The molar absorption coefficient of a compound can be measured using a well-publicly known method. For example, it is preferable to carry out a measurement at a concentration of 0.01 g/L using an ethyl acetate solvent with an ultraviolet-visible spectrophotometer (Cary-5 spectrophotometer manufactured by Varian Medical Systems, Inc.).

As a photoradical polymerization initiator, well-known compounds can be optionally used. Examples thereof include a halogenated hydrocarbon derivative (for example, a compound having a triazine skeleton, a compound having an oxadiazole skeleton, or a compound having a trihalomethyl group), an acylphosphine compound such as an acylphosphine oxide, hexaarylbiimidazole, an oxime compound such as an oxime derivative, an organic peroxide, a thio compound, a ketone compound, an aromatic onium salt, a keto oxime ether, an α-amino ketone compound such as aminoacetophenone, an α-hydroxy ketone compound such as hydroxyacetophenone, an azo-based compound, an azide compound, a metallocene compound, an organic boron compound, and an iron arene complex. With regard to details thereof, reference can be made to the description of paragraphs 0165 to 0182 of JP2016-027357A and paragraphs 0138 to 0151 of WO2015/199219A, the contents of which are incorporated in the present specification. In addition, examples thereof include the compounds described in paragraphs 0065 to 0111 of JP2014-130173A and JP6301489B, the peroxide-based photopolymerization initiator described in MATERIAL STAGE 37 to 60 p, vol. 19, No. 3, 2019, the photopolymerization initiator described in WO2018/221177A, the photopolymerization initiator described in WO2018/110179A, the photopolymerization initiator described in JP2019-043864A, the photopolymerization initiator described in JP2019-044030A, and the peroxide-based initiator described in JP2019-167313A, the contents of which are incorporated in the present specification.

Examples of the ketone compound include compounds described in paragraph 0087 of JP2015-087611A, the content of which is incorporated in the present specification. As a commercially available product thereof, KAYACURE DETX-S (manufactured by Nippon Kayaku Co., Ltd.) is also suitably used.

In one embodiment of the present invention, a hydroxyacetophenone compound, an aminoacetophenone compound, and an acylphosphine compound can be suitably used as the photoradical polymerization initiator. More specifically, for example, the aminoacetophenone-based initiator described in JP1998-291969A (JP-H10-291969A) and the acylphosphine oxide-based initiator described in JP4225898B can be used, the contents of which are incorporated in the present specification.

As the α-hydroxy ketone-based initiator, Omnirad 184, Omnirad 1173, Omnirad 2959, Omnirad 127 (all of which are manufactured by IGM Resins B.V.), IRGACURE 184 (IRGACURE is a registered trademark), DAROCUR 1173, IRGACURE 500, IRGACURE-2959, and IRGACURE 127 (all of which are manufactured by BASF SE) can be used.

As the α-amino ketone-based initiator, Omnirad 907, Omnirad 369, Omnirad 369E, Omnirad 379EG (all of which are manufactured by IGM Resins B.V.), IRGACURE 907, IRGACURE 369, and IRGACURE 379 (all of which are manufactured by BASF SE) can be used.

As the aminoacetophenone-based initiator, the acylphosphine oxide-based initiator, and the metallocene compound, it is also possible to suitably use, for example, the compounds described in paragraphs 0161 to 0163 of WO2021/112189A. The content thereof is incorporated in the present specification.

Examples of the more preferred photoradical polymerization initiator include an oxime compound. In a case where an oxime compound is used, exposure latitude can be more effectively improved. The oxime compound is particularly preferable since the oxime compound has a wide exposure latitude (a wide exposure margin) and also works as a photocuring accelerator.

Specific examples of the oxime compound include the compounds described in JP2001-233842A, the compounds described in JP2000-080068A, the compounds described in JP2006-342166A, the compounds described in J. C. S. Perkin II (1979, pp. 1653-1660), the compounds described in J. C. S. Perkin II (1979, pp. 156-162), the compounds described in Journal of Photopolymer Science and Technology (1995, pp. 202-232), the compounds described in JP2000-066385A, the compounds described in JP2004-534797A, the compounds described in JP2017-019766A, the compounds described in JP6065596B, the compounds described in WO2015/152153A, the compounds described in WO2017/051680A, the compounds described in JP2017-198865A, the compounds described in paragraph Nos. 0025 to 0038 of WO2017/164127A, and the compounds described in WO2013/167515A, the content of which is incorporated in the present specification.

Examples of the preferred oxime compound include compounds having the following structures, 3-(benzoyloxy(imino))butan-2-one, 3-(acetoxy(imino))butan-2-one, 3-(propionyloxy(imino))butan-2-one, 2-(acetoxy(imino))pentan-3-one, 2-(acetoxy(imino))-1-phenylpropan-1-one, 2-(benzoyloxy(imino))-1-phenylpropan-1-one, 3-((4-toluenesulfonyloxy)imino)butan-2-one, and 2-(ethoxycarbonyloxy(imino))-1-phenylpropan-1-one. In the resin composition, it is preferable to use an oxime compound, particularly as a photoradical polymerization initiator. The oxime compound as a photoradical polymerization initiator has a linking group of >C═N—O—C(═O)— in the molecule.

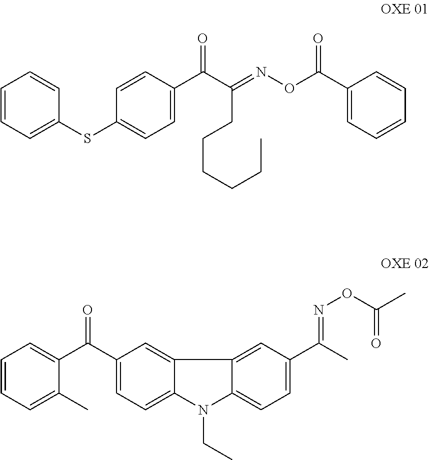 [SMILES: CCCCCC/C(=N\OC(=O)c1ccccc1)C(=O)c1ccc(Sc2ccccc2)cc1 ; CCn1c2ccc(C(=O)c3ccccc3C)cc2c2cc(/C(C)=N/OC(C)=O)ccc21]

Examples of the commercially available product of the oxime compound include IRGACURE OXE 01, IRGACURE OXE 02, IRGACURE OXE 03, and IRGACURE OXE 04 (all of which are manufactured by BASF SE), ADEKA OPTOMER N-1919 (manufactured by ADEKA Corporation, the photoradical polymerization initiator 2 described in JP2012-014052A), TR-PBG-304 and TR-PBG-305 (all of which are manufactured by Changzhou Tronly New Electronic Materials Co., Ltd.), ADEKA ARKLS NCI-730, NCI-831, and ADEKA ARKLS NCI-930 (all of which are manufactured by ADEKA Corporation), DFI-091 (manufactured by DAITO CHEMIX Co., Ltd.), and SpeedCure PDO (manufactured by SARTOMER ARKEMA). In addition, oxime compounds having the following structures can also be used.

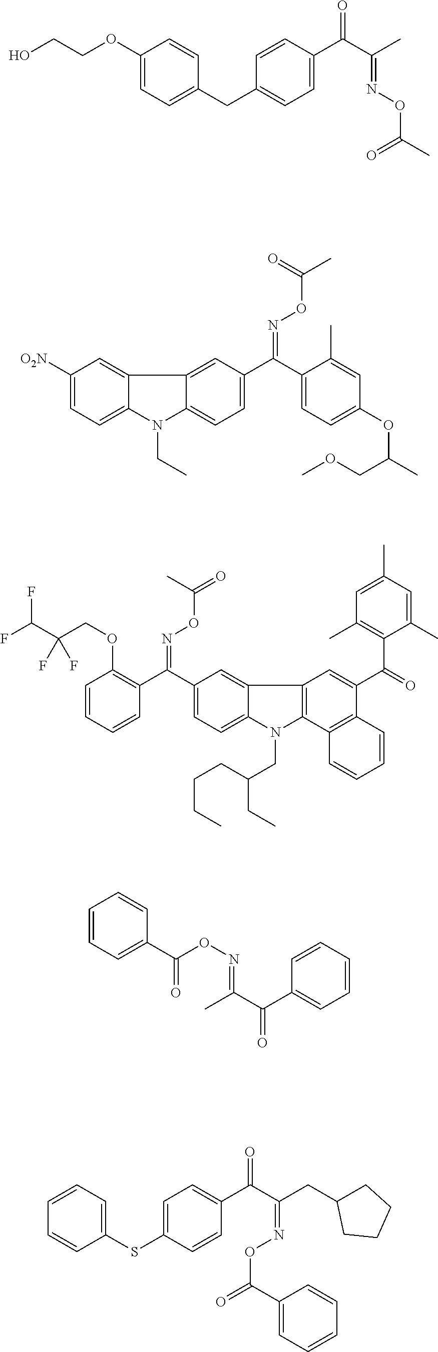 [SMILES: CC(=O)O/N=C(\C)C(=O)c1ccc(Cc2ccc(OCCO)cc2)cc1 ; CCn1c2ccc(/C(=N/OC(C)=O)c3ccc(OC(C)COC)cc3C)cc2c2cc([N+](=O)[O-])ccc21 ; CCCCC(CC)Cn1c2ccc(/C(=N\OC(C)=O)c3ccccc3OCC(F)(F)C(F)F)cc2c2cc(C(=O)c3c(C)cc(C)cc3C)c3ccccc3c21 ; C/C(=N\OC(=O)c1ccccc1)C(=O)c1ccccc1 ; O=C(O/N=C(/CC1CCCC1)C(=O)c1ccc(Sc2ccccc2)cc1)c1ccccc1]

As the photoradical polymerization initiator, it is also possible to use, for example, the oxime compounds having a fluorene ring, which are described in paragraphs 0169 to 0171 of WO2021/112189A, an oxime compound having a skeleton in which at least one benzene ring of a carbazole ring is a naphthalene ring, or an oxime compound having a fluorine atom.

In addition, it is also possible to use the oxime compounds having a nitro group, which are described in paragraphs 0208 to 0210 of WO2021/020359A, an oxime compound having a benzofuran skeleton, or an oxime compound in which a substituent having a hydroxy group is bonded to a carbazole skeleton. The content thereof is incorporated in the present specification.

As the photopolymerization initiator, an oxime compound having an aromatic ring group Ar^{OX1} in which an electron withdrawing group is introduced into an aromatic ring (hereinafter, also referred to as an oxime compound OX) can also be used. Examples of the electron withdrawing group contained in the aromatic ring group Ar^{OX1} include an acyl group, a nitro group, a trifluoromethyl group, an alkylsulfinyl group, an arylsulfinyl group, an alkylsulfonyl group, an arylsulfonyl group, and a cyano group. Here, an acyl group or a nitro group is preferable, and due to the reason that a film having excellent light resistance is easily formed, an acyl group is more preferable, and a benzoyl group is still more preferable. The benzoyl group may have a substituent. The substituent is preferably a halogen atom, a cyano group, a nitro group, a hydroxy group, an alkyl group, an alkoxy group, an aryl group, an aryloxy group, a heterocyclic group, a heterocyclic oxy group, an alkenyl group, an alkylsulfanyl group, an arylsulfanyl group, an acyl group, or an amino group, more preferably an alkyl group, an alkoxy group, an aryl group, an aryloxy group, a heterocyclic oxy group, an alkylsulfanyl group, an arylsulfanyl group, or an amino group, and still more preferably an alkoxy group, an alkylsulfanyl group, or an amino group.

The oxime compound OX is preferably at least one selected from a compound represented by Formula (OX1) or a compound represented by Formula (OX2), and more preferably a compound represented by Formula (OX2).

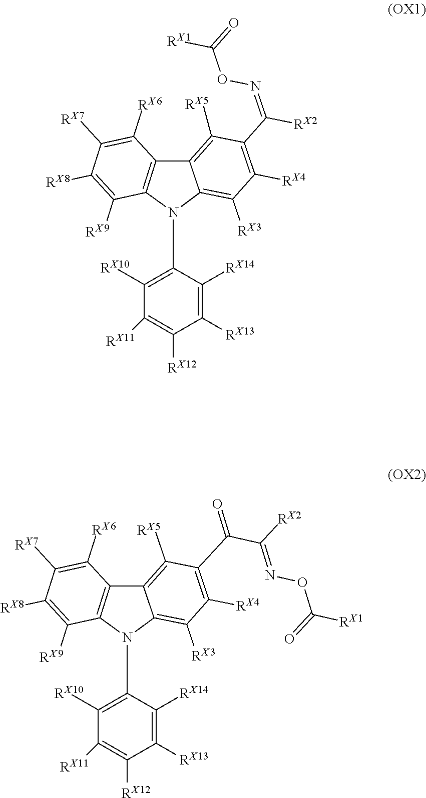

In the formulae, R^{X1} represents an alkyl group, an alkenyl group, an alkoxy group, an aryl group, an aryloxy group, a heterocyclic group, a heterocyclic oxy group, an alkylsulfanyl group, an arylsulfanyl group, an alkylsulfinyl group, an arylsulfinyl group, an alkylsulfonyl group, an arylsulfonyl group, an acyl group, an acyloxy group, an amino group, a phosphinoyl group, a carbamoyl group, or a sulfamoyl group,
R^{X2} represents an alkyl group, an alkenyl group, an alkoxy group, an aryl group, an aryloxy group, a heterocyclic group, or a heterocyclic oxy group, an alkylsulfanyl group, an arylsulfanyl group, an alkylsulfinyl group, an arylsulfinyl group, an alkylsulfonyl group, an arylsulfonyl group, an acyloxy group, or an amino group, and
R^{X3} to R^{X14} each independently represent a hydrogen atom or a substituent.

However, at least one of R^{X10}, . . . , or R^{X14} is an electron withdrawing group.

In the above Formulae, it is preferable that R^{X12} is an electron withdrawing group, and R^{X10}, R^{X11}, R^{X13}, and R^{X14} are a hydrogen atom.

Specific examples of the oxime compound OX include the compounds described in paragraph Nos. 0083 to 0105 of JP4600600B, the content of which is incorporated in the present specification.

Examples of the particularly preferred oxime compound include the oxime compound having a specific substituent described in JP2007-269779A and the oxime compound having a thioaryl group described in JP2009-191061A, the contents of which are incorporated in the present specification.

From the viewpoint of exposure sensitivity, the photoradical polymerization initiator is preferably a compound selected from the group consisting of a trihalomethyltriazine compound, a benzyl dimethyl ketal compound, an α-hydroxy ketone compound, an α-aminoketone compound, an acylphosphine compound, a phosphine oxide compound, a metallocene compound, an oxime compound, a triaryl imidazole dimer, an onium salt compound, a benzothiazole compound, a benzophenone compound, an acetophenone compound and a derivative thereof, a cyclopentadiene-benzene-iron complex and a salt thereof, a halomethyl oxadiazole compound, and a 3-aryl substituted coumarin compound.

In addition, the photoradical polymerization initiator is a trihalomethyltriazine compound, an α-aminoketone compound, an acylphosphine compound, a phosphine oxide compound, a metallocene compound, an oxime compound, a triaryl imidazole dimer, an onium salt compound, a benzophenone compound, or an acetophenone compound. It is more preferably at least one compound selected from the group consisting of a trihalomethyltriazine compound, an α-aminoketone compound, a metallocene compound, an oxime compound, a triaryl imidazole dimer, and a benzophenone compound, and still more preferably a metallocene compound or an oxime compound.

As the photoradical polymerization initiator, the compounds described in paragraphs 0175 to 0179 of WO2021/020359A and the compounds described in paragraphs 0048 to 0055 of WO2015/125469A can also be used, and the contents of which are incorporated in the present specification.

As the photoradical polymerization initiator, a photoradical polymerization initiator which is difunctional or tri- or higher functional may be used. In a case where such a photoradical polymerization initiator is used, two or more radicals are generated from one molecule of the photoradical polymerization initiator, and thus good sensitivity is obtained. Further, in a case where a compound having an asymmetric structure is used, the crystallinity is reduced, the solubility in a solvent or the like is improved, and the compound is hardly precipitated over time, which makes it possible to improve the temporal stability of the resin composition. Specific examples of the photoradical polymerization initiator which is difunctional or tri- or higher functional include dimers of the oxime compounds described in JP2010-527339A, JP2011-524436A, WO2015/004565A, paragraph Nos. 0407 to 0412 of JP2016-532675A, and paragraph Nos. 0039 to 0055 of WO2017/033680A, the compound (E) and compound (G) described in JP2013-522445A, Cmpd 1 to 7 described in WO2016/034963A, the oxime ester photoinitiators described in paragraph No. 0007 of JP2017-523465A, the photoinitiators described in paragraph Nos. 0020 to 0033 of JP2017-167399A, the photopolymerization initiator (A) described in paragraph Nos. 0017 to 0026 of JP2017-151342A, and the oxime ester photoinitiator described in JP6469669B, the contents of which are incorporated in the present specification.

In a case where the resin composition contains a photopolymerization initiator, the content thereof is preferably 0.1% to 30% by mass, more preferably 0.1% to 20% by mass, still more preferably 0.5% to 15% by mass, and even still more preferably 1.0% to 10% by mass with respect to the total solid content of the resin composition. Only one kind of photopolymerization initiator may be contained, or two or more kinds thereof may be contained. In a case where two or more kinds of photopolymerization initiators are contained, the total amount thereof is preferably within the above-described range.

It is noted that since the photopolymerization initiator may also function as a thermal polymerization initiator, crosslinking with the photopolymerization initiator may be further allowed to proceed by heating an oven, a hot plate, or the like.

### [Sensitizing Agent]

The resin composition may contain a sensitizing agent. The sensitizing agent absorbs a specific radioactive ray to be in an electronically excited state. The sensitizing agent in the electronically excited state is brought into contact with a thermal radical polymerization initiator, a photoradical polymerization initiator, or the like, to cause actions such as electron migration, energy transfer, and heat generation. As a result, the thermal radical polymerization initiator and the photoradical polymerization initiator undergo a chemical change and decompose to generate a radical, an acid, or a base.

In addition, as a usable sensitizing agent, a benzophenone-based, a Michler's ketone-based, a coumarin-based, a pyrazole azo-based, an anilino azo-based, a triphenylmethane-based, an anthraquinone-based, an anthracene-based, an anthrapylidene-based, a benzylidene-based, an oxonol-based, a pyrazolotriazole azo-based, a pyridone azo-based, a cyanine-based, a phenothiazine-based, a pyrrolopyrazole azomethine-based, a xanthene-based, a phthalocyanine-based, a benzopyran-based, and an indigo-based compound can be used.

Examples of the sensitizing agent include, Michler's ketone, 4,4′-bis(diethylamino)benzophenone, 2,5-bis(4′-diethylaminobenzal)cyclopentane, 2,6-bis(4′-diethylaminobenzal)cyclohexanone, 2,6-bis(4′-diethylaminobenzal)-4-methylcyclohexanone, 4,4′-bis(dimethylamino)chalcone, 4,4′-bis(diethylamino)chalcone, p-dimethylaminocinnamylidene indanone, p-dimethylamino benzylidene indanone, 2-(p-dimethylaminophenylbiphenylene)-benzothiazole, 2-(p-dimethylaminophenylvinylene)benzothiazole, 2-(p-dimethylaminophenylvinylene)isonaphtothiazole, 1,3-bis(4′-dimethylaminobenzal)acetone, 1,3-bis(4′-diethylaminobenzal)acetone, 3,3′-carbonyl-bis(7-diethylaminocoumarin), 3-acetyl-7-dimethylaminocoumarin, 3-ethoxycarbonyl-7-dimethylaminocoumarin, 3-benzyloxycarbonyl-7-dimethylaminocoumarin, 3-methoxycarbonyl-7-diethylaminocoumarin, 3-ethoxycarbonyl-7-diethylaminocoumarin (ethyl 7-(diethylamino)coumarin-3-carboxylate), N-phenyl-N′-ethylethanolamine, N-phenyldiethanolamine, N-p-tolyldiethanolamine, N-phenylethanolamine, 4-morpholinobenzophenone, isoamyl dimethylaminobenzoate, isoamyl diethylaminobenzoate, 2-mercaptobenzimidazole, 1-phenyl-5-mercaptotetrazole, 2-mercaptobenzothiazole, 2-(p-dimethylaminostyryl)benzoxazole, 2-(p-dimethylaminostyryl)benzothiazole, 2-(p-dimethylaminostyryl)naphtho (1,2-d)thiazole, 2-(p-dimethylaminobenzoyl)styrene, diphenylacetamide, benzanilide, N-methylacetanilide, and 3′,4′-dimethylacetanilide.

In addition, other sensitizing dyes may be used.

For details of the sensitizing dye, reference can be made to the description in paragraphs 0161 to 0163 of JP2016-027357A, the content of which is incorporated in the present specification.

In a case where the resin composition contains a sensitizing agent, the content of the sensitizing agent is preferably 0.01% to 20% by mass, more preferably 0.1% to 15% by mass, and still more preferably 0.5% to 10% by mass with respect to the total solid content of the resin composition. One kind of sensitizing agent may be used alone, or two or more kinds thereof may be used in combination.

### [Chain Transfer Agent]

The resin composition according to the embodiment of the present invention may contain a chain transfer agent. The chain transfer agent is defined, for example, in Polymer Dictionary, 3rd Edition, pp. 683 to 684 (edited by The Society of Polymer Science, 2005). As the chain transfer agent, for example, the following compound is used; a group of compounds having —S—S—, —SO₂—S—, —N—O—, SH, PH, SiH, or GeH in the molecule, or a dithiobenzoate compound, a trithiocarbonate compound, dithiocarbamate, or a xanthate compound, which has a thiocarbonylthio group that is used for the reversible addition fragmentation chain transfer (RAFT) polymerization. These can donate hydrogen to a low active radical to generate a radical or can be oxidized and then deprotonated to generate a radical. In particular, a thiol compound can be preferably used.

In addition, as the chain transfer agent, the compounds described in paragraphs 0152 and 0153 of WO2015/199219A can also be used, the content of which is incorporated in the present specification.

In a case where the resin composition has a chain transfer agent, the content of the chain transfer agent is preferably 0.01 to 20 parts by mass, more preferably 0.1 to 10 parts by mass, and still more preferably 0.5 to 5 parts by mass, with respect to 100 parts by mass of the total solid content of the resin composition. Only one kind of chain transfer agent may be used alone, or two or more kinds thereof may be used. In a case where two or more kinds of chain transfer agents are used, the total thereof is preferably within the above-described range.

In addition, a photoacid generator may be used as the photopolymerization initiator. The photoacid generator is preferably a photoacid generator that generates a radical.

Specifically, the compound is preferably a compound that absorbs light, decomposes the absorbed light to generate a radical, and abstracts hydrogen from a solvent, an acid generator itself, or the like to generate an acid.

Examples of the photoacid generator include a quinone diazide compound, an oxime sulfonate compound, an organic halogenated compound, an organic borate compound, a disulfone compound, and an onium salt, and an onium salt is preferable.

Examples of the onium salt include a diazonium salt, a phosphonium salt, a sulfonium salt, and an iodonium salt.

Further, the onium salt is a salt of a cation having an onium structure and an anion, and the cation and the anion may be or may not be bonded through a covalent bond.

That is, the onium salt may be an intramolecular salt having a cation moiety and an anion moiety in the same molecular structure, or may be an intermolecular salt in which a cation molecule and an anion molecule, which are separate molecules, are ionically bonded; however, an intermolecular salt is preferred. Further, in the composition according to the embodiment of the present invention, the cation moiety or the cation molecule and the anion moiety or the anion molecule may be bonded by the ionic bonding or be dissociated from each other.

### [Sulfonium Salt]

In the present invention, the sulfonium salt means a salt of a sulfonium cation and an anion.

### —Sulfonium Cation—

The sulfonium cation is preferably a tertiary sulfonium cation and more preferably a triaryl sulfonium cation.

In addition, the sulfonium cation is preferably a cation represented by Formula (103).

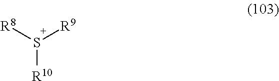

In Formula (103), R⁸ to R¹⁰ each independently represent a hydrocarbon group.

R⁸ to R¹⁰ are each independently preferably an alkyl group or an aryl group, more preferably an alkyl group having 1 to 10 carbon atoms or an aryl group having 6 to 12 carbon atoms, still more preferably an aryl group having 6 to 12 carbon atoms, and even still more preferably a phenyl group.

R⁸ to R¹⁰ may have a substituent, and examples of the substituent include a hydroxy group, an aryl group, an alkoxy group, an aryloxy group, an arylcarbonyl group, an alkylcarbonyl group, an alkoxycarbonyl group, an aryloxycarbonyl group, and an acyloxy group. Among these, R⁸ to R¹⁰ preferably have an alkyl group or an alkoxy group as the substituent, more preferably have a branched alkyl group or an alkoxy group, and still more preferably have a branched alkyl group having 3 to 10 carbon atoms or an alkoxy group having 1 to 10 carbon atoms.

R⁸ to R¹⁰ may be the same group or different groups; however, from the viewpoint of synthesis compatibility, R⁸ to R¹⁰ are preferably the same group.

### —Anion—

The anion is not particularly limited, and may be selected in consideration of the acid generated. Examples of the anion include boron-based anions such as B(C₆F₅)₄⁻ and BF₄⁻, phosphorus-based anions such as (Rf)ₙPF₆₋ₙ⁻, PF₃(C₂F₅)₃⁻ and PF₆⁻, antimony-based anions such as SbF₆⁻, and other carboxylate anions, sulfonate anions, and the like.

### [Iodonium Salt]

In the present invention, the iodonium salt means a salt of an iodonium cation and an anion. Examples of the anion include the same anions as those in the sulfonium salt described above, and the same applies to the preferred aspect.

### —Iodonium Cation—

The iodonium cation is preferably a diaryl iodonium cation.

In addition, the iodonium cation is preferably a cation represented by Formula (104).

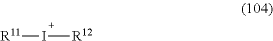

In Formula (104), R¹¹ and R¹² each independently represent a hydrocarbon group.

R¹¹ and R¹² are each independently preferably an alkyl group or an aryl group, more preferably an alkyl group having 1 to 10 carbon atoms or an aryl group having 6 to 12 carbon atoms, still more preferably an aryl group having 6 to 12 carbon atoms, and even still more preferably a phenyl group.

R¹¹ and R¹² may have a substituent, and examples of the substituent include a hydroxy group, an aryl group, an alkoxy group, an aryloxy group, an arylcarbonyl group, an alkylcarbonyl group, an alkoxycarbonyl group, an aryloxycarbonyl group, and an acyloxy group. Among these, R¹¹ to R¹² preferably have an alkyl group or an alkoxy group as the substituent, more preferably have a branched alkyl group or an alkoxy group, and still more preferably have a branched alkyl group having 3 to 10 carbon atoms or an alkoxy group having 1 to 10 carbon atoms.

R¹¹ and R¹² may be the same group or different groups; however, from the viewpoint of synthesis compatibility, R¹¹ and R¹² are preferably the same group.

### [Phosphonium Salt]

In the present invention, the phosphonium salt means a salt of a phosphonium cation and an anion. Examples of the anion include the same anions as those in the sulfonium salt described above, and the same applies to the preferred aspect.

### —Phosphonium Cation—

The phosphonium cation is preferably a quaternary phosphonium cation, and examples thereof include a tetraalkylphosphonium cation and a triarylmonoalkylphosphonium cation.

In addition, the phosphonium cation is preferably a cation represented by Formula (105).

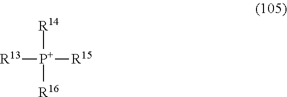

In Formula (105), R¹³ to R¹⁶ each independently represent a hydrogen atom or a hydrocarbon group.

R¹³ to R¹⁶ are each independently preferably an alkyl group or an aryl group, more preferably an alkyl group having 1 to 10 carbon atoms or an aryl group having 6 to 12 carbon atoms, still more preferably an aryl group having 6 to 12 carbon atoms, and even still more preferably a phenyl group.

R¹³ to R¹⁶ may have a substituent, and examples of the substituent include a hydroxy group, an aryl group, an alkoxy group, an aryloxy group, an arylcarbonyl group, an alkylcarbonyl group, an alkoxycarbonyl group, an aryloxycarbonyl group, and an acyloxy group. Among these, R¹³ to R¹⁶ preferably have an alkyl group or an alkoxy group as the substituent, more preferably have a branched alkyl group or an alkoxy group, and still more preferably have a branched alkyl group having 3 to 10 carbon atoms or an alkoxy group having 1 to 10 carbon atoms.

R¹³ to R¹⁶ may be the same group or different groups; however, from the viewpoint of synthesis compatibility, R¹³ to R¹⁶ are preferably the same group.

The content of the photoacid generator is preferably 0.1% to 20% by mass, more preferably 0.5% to 18% by mass, still more preferably 0.5% to 10% by mass, even more preferably 0.5% to 3% by mass, and even still more preferably 0.5% to 1.2% by mass, with respect to the total solid content of the resin composition.

One kind of photoacid generator may be used alone, or a plurality of kinds thereof may be used in combination. In the case of the combination of a plurality of kinds, it is preferable that the total amount thereof is within the above range.

In addition, in order to impart photosensitivity to a desired light source, it is also preferable to use a sensitizing agent in combination.

In addition, one of the preferred aspects of the present invention is that the resin composition according to the embodiment of the present invention contains two or more kinds of polymerization initiators as the polymerization initiator.

Specifically, the resin composition according to the embodiment of the present invention preferably contains a photopolymerization initiator and a thermal polymerization initiator described later, or contains the above-described photoradical polymerization initiator and the above-described photoacid generator.

By containing the photopolymerization initiator and the thermal polymerization initiator described later, pattern formation by exposure is possible, and radical polymerization is also likely to proceed during curing in the heating step described later, and thus the performance such as chemical resistance may be improved.

In the content ratio in a case where the photopolymerization initiator and the thermal polymerization initiator described later are contained, the content of the thermal polymerization initiator is preferably 20% to 70% by mass and more preferably 30% to 60% by mass with respect to the total content of the photopolymerization initiator and the thermal polymerization initiator.

The performance such as resolution may be improved by containing a photoradical polymerization initiator and a photoacid generator.

In the content ratio in a case where the photopolymerization initiator and the photoacid generator are contained, the content of the photoacid generator is preferably 20% to 70% by mass, and more preferably 30% to 60% by mass with respect to the total content of the photopolymerization initiator and the photoacid generator.

### [Thermal Polymerization Initiator]

Examples of the thermal polymerization initiator include a thermal radical polymerization initiator. The thermal radical polymerization initiator is a compound that generates a radical by heat energy and initiates or accelerates a polymerization reaction of a compound having polymerization properties. In a case where a thermal radical polymerization initiator is added, the polymerization reaction of the resin and the polymerizable compound can be allowed to proceed, and thus the solvent resistance can be further improved.

Specific examples of thermal radical polymerization initiator include compounds described in paragraphs 0074 to 0118 of JP2008-063554A, the content of which is incorporated in the present specification.

In a case where a thermal polymerization initiator is contained, the content thereof is preferably 0.1% to 30% by mass, more preferably 0.1% to 20% by mass, and still more preferably 0.5% to 15% by mass with respect to the total solid content of the resin composition. The resin composition may contain only one kind of thermal polymerization initiator, or may contain two or more kinds thereof. In a case where two or more kinds of thermal polymerization initiators are contained, the total amount thereof is preferably within the above-described range.

### <Base Generator>

The resin composition according to the embodiment of the present invention may contain a base generator. Here, the base generator is a compound that is capable of generating a base under a physical or chemical action. Examples of the preferred base generator include a thermal-base generator and a photobase generator.

In particular, in a case where the resin composition contains a precursor of a cyclization resin, it is preferable that the resin composition contains a base generator. In a case where the resin composition contains a thermal-base generator, it is possible to, for example, accelerate the cyclization reaction of the precursor by heating, whereby the mechanical properties and chemical resistance of the cured substance are improved and for example, the performance as an interlayer insulating film for a re-distribution layer, included in a semiconductor package, is improved.

The base generator may be an ionic base generator or may be a nonionic base generator. Examples of the base that is generated from the base generator include a secondary amine and a tertiary amine.

The base generator is not particularly limited, and a publicly known base generator can be used. Examples of the publicly known base generator include a carbamoyloxime compound, a carbamoylhydroxylamine compound, a carbamic acid compound, a formamide compound, an acetamide compound, a carbamate compound, a benzylcarbamate compound, a nitrobenzylcarbamate compound, a sulfonamide compound, an imidazole derivative compound, an aminimide compound, a pyridine derivative compound, an α-aminoacetophenone derivative compound, a quaternary ammonium salt derivative compound, an iminium salt, a pyridinium salt, an α-lactone ring derivative compound, a phthalimide derivative compound, and an acyloxyimino compound.

Specific examples of the nonionic base generator include the compounds described in paragraphs 0249 to 0275 of WO2022/145355A. The above description is incorporated in the present specification.

Examples of the base generator include the following compounds; however, the base generator is not limited thereto.

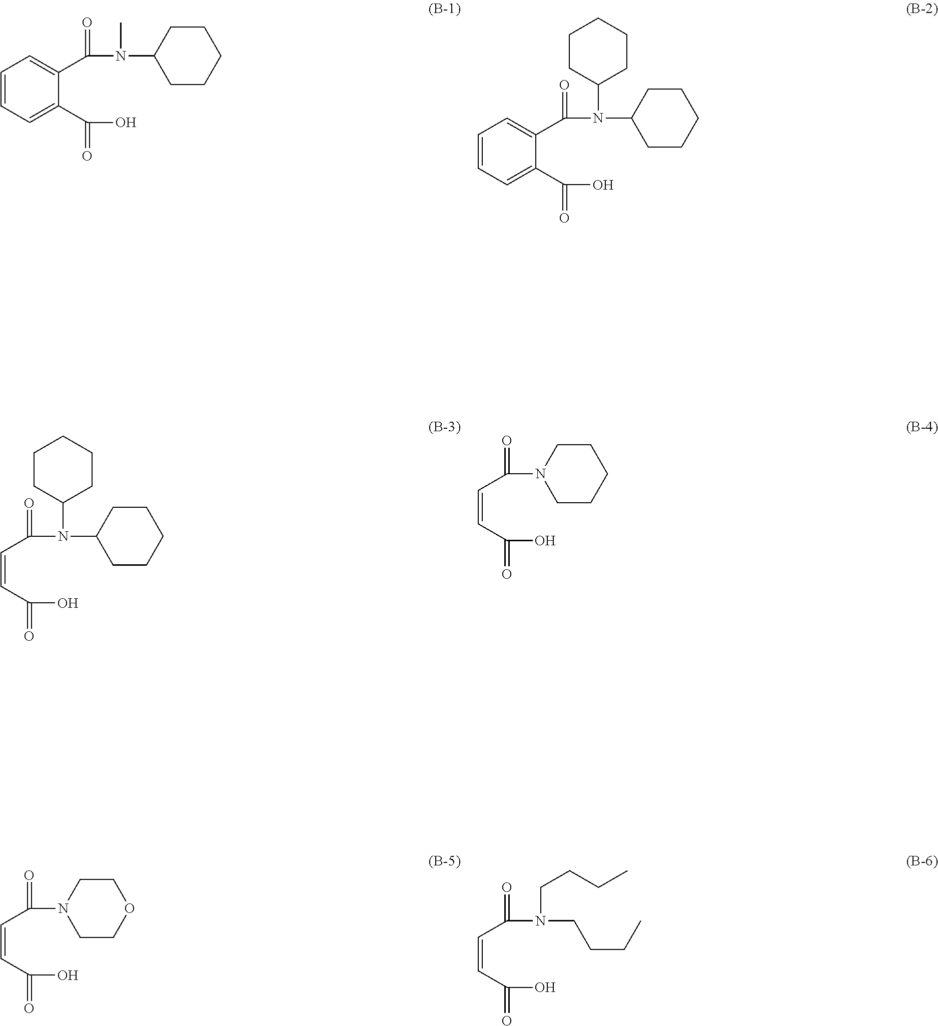 [SMILES: CN(C(=O)c1ccccc1C(=O)O)C1CCCCC1 ; O=C(O)c1ccccc1C(=O)N(C1CCCCC1)C1CCCCC1 ; O=C(O)/C=C\C(=O)N(C1CCCCC1)C1CCCCC1 ; O=C(O)/C=C\C(=O)N1CCCCC1 ; O=C(O)/C=C\C(=O)N1CCOCC1 ; CCCCN(CCCC)C(=O)/C=C\C(=O)O]

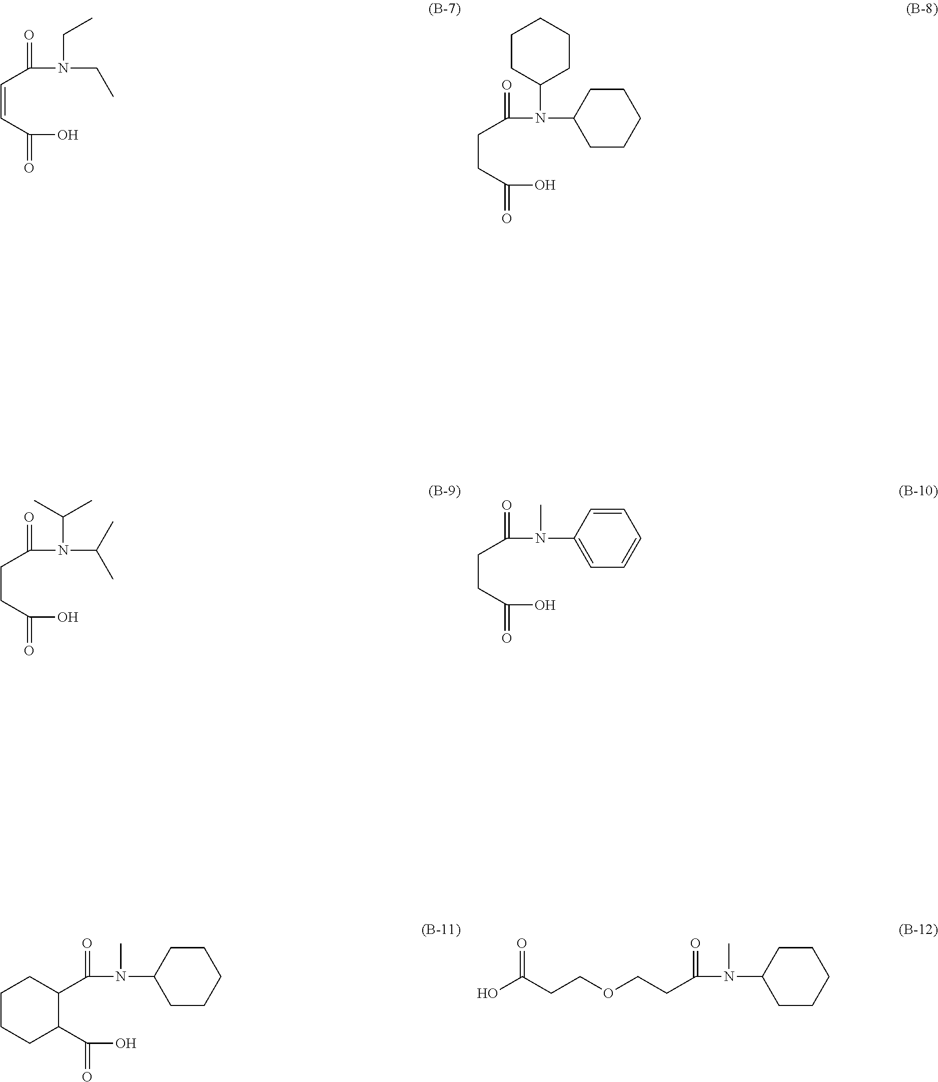 [SMILES: CCN(CC)C(=O)/C=C\C(=O)O ; O=C(O)CCC(=O)N(C1CCCCC1)C1CCCCC1 ; CC(C)N(C(=O)CCC(=O)O)C(C)C ; CN(C(=O)CCC(=O)O)c1ccccc1 ; CN(C(=O)C1CCCCC1C(=O)O)C1CCCCC1 ; CN(C(=O)CCOCCC(=O)O)C1CCCCC1]

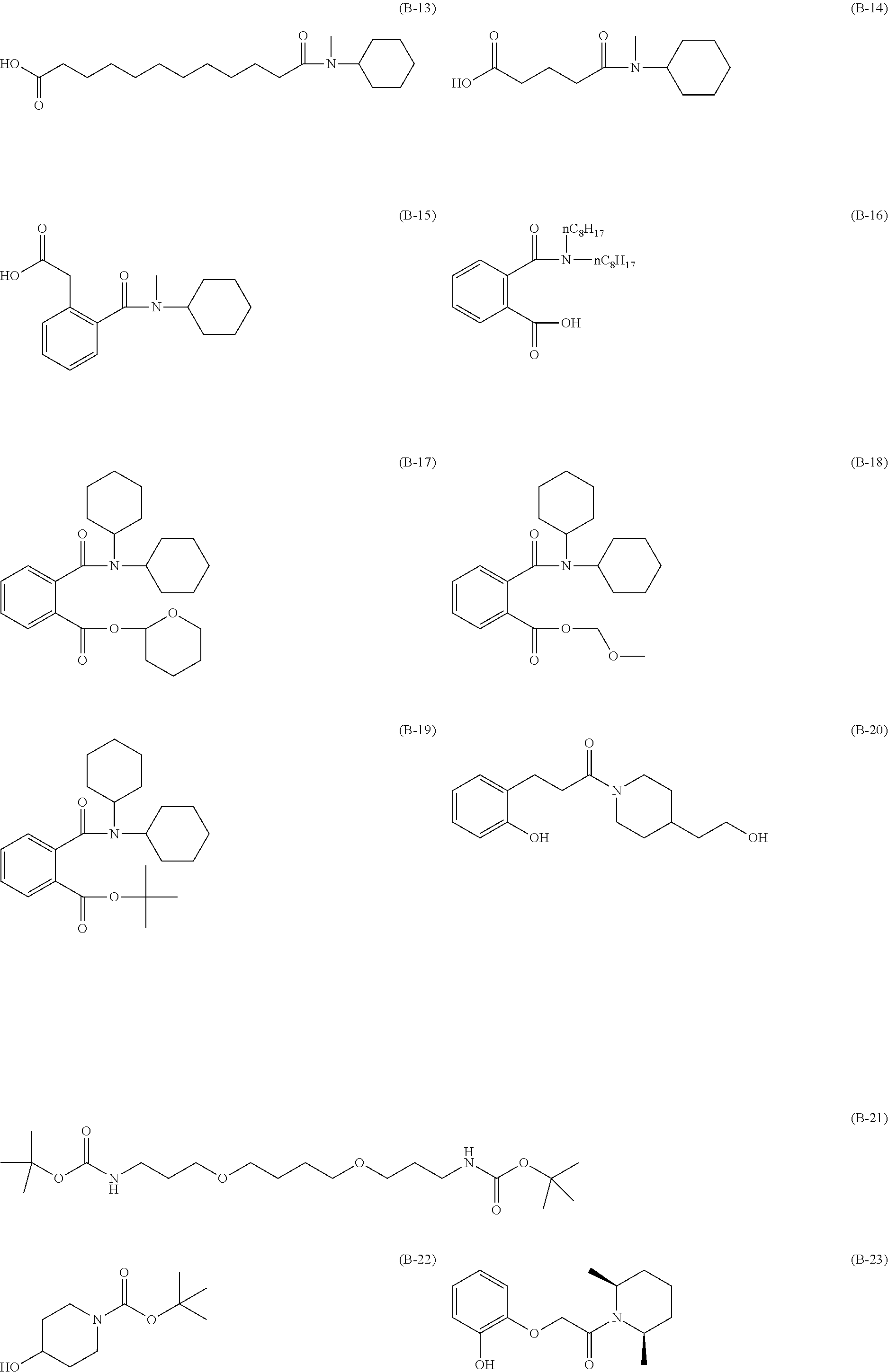

The molecular weight of the nonionic base generator is preferably 800 or less, more preferably 600 or less, and still more preferably 500 or less. The lower limit thereof is preferably 100 or more, more preferably 200 or more, and still more preferably 300 or more.

Examples of the specific preferred compound of the ionic base generator include the compounds described in paragraphs 0148 to 0163 of WO2018/038002A.

Specific examples of the ammonium salt include the following compounds; however, the ammonium salt is not limited thereto.

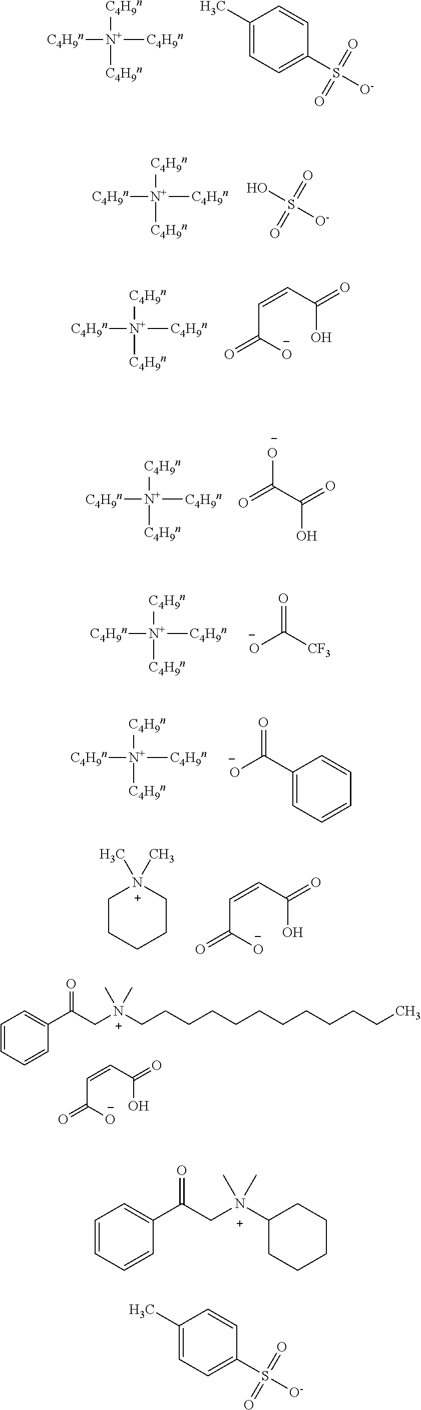

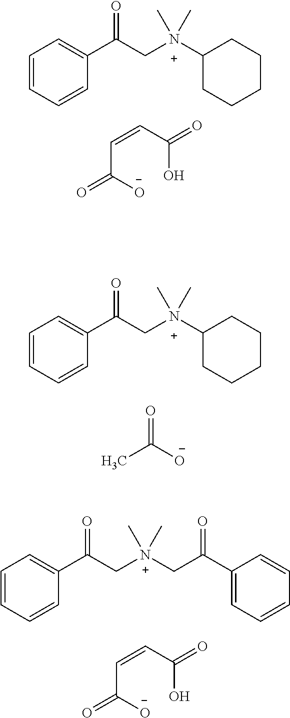

Specific examples of the iminium salt include the following compounds; however, the iminium salt is not limited thereto.

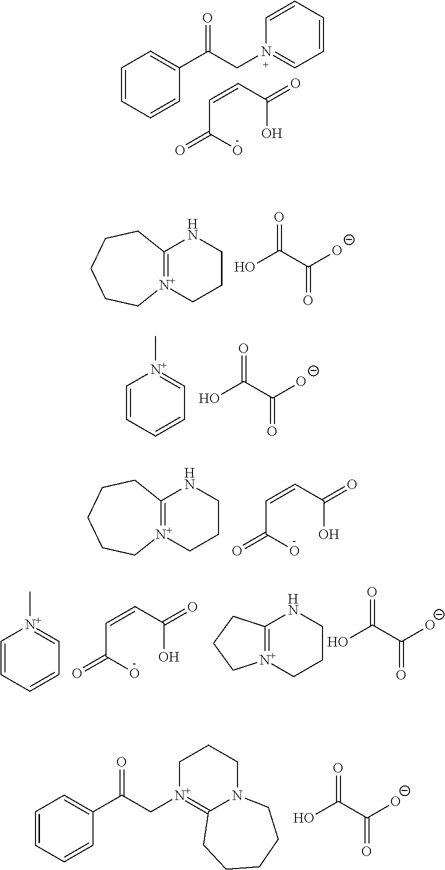

In addition, from the viewpoint of storage stability and generation of a base by deprotection during curing, the base generator is preferably an amine in which an amino group is protected by a t-butoxycarbonyl group.

Examples of the amine compound protected by a t-butoxycarbonyl group include ethanolamine, 3-amino-1-propanol, 1-amino-2-propanol, 2-amino-1-propanol, 4-amino-1-butanol, 2-amino-1-butanol, 1-amino-2-butanol, 3-amino-2,2-dimethyl-1-propanol, 4-amino-2-methyl-1-butanol, valinol, 3-amino-1,2-propanediol, 2-amino-1,3-propanediol, tyramine, norephedrine, 2-amino-1-phenyl-1,3-propanediol, 2-aminocyclohexanol, 4-aminocyclohexanol, 4-aminocyclohexanethanol, 4-(2-aminoethyl)cyclohexanol, N-methylethanolamine, 3-(methylamino)-1-propanol, 3-(isopropylamino)propanol, N-cyclohexylethanolamine, α-[2-(methylamino)ethyl]benzyl alcohol, diethanolamine, diisopropanolamine, 3-pyrrolidinol, 2-pyrrolidinomethanol, 4-hydroxypiperidine, 3-hydroxypiperidine, 4-hydroxy-4-phenylpiperidine, 4-(3-hydroxyphenyl)piperidine, 4-piperidinemethanol, 3-piperidinemethanol, 2-piperidinemethanol, 4-piperidinetanol, 2-piperidinetanol, 2-(4-piperidyl)-2-propanol, 1,4-butanol bis(3-aminopropyl)ether, 1,2-bis(2-aminoethoxy)ethane, 2,2′-oxybis(ethylamine), 1,14-diamino-3,6,9,12-tetraoxatetradecane, 1-aza-15-crown 5-ether, diethylene glycol bis(3-aminopropyl)ether, 1,11-diamino-3,6,9-trioxaundecane, or a compound in which an amino group of an amino acid and a derivative thereof is protected by a t-butoxycarbonyl group, but the present invention is not limited to these examples.

In a case where the resin composition contains a base generator, the content of the base generator is preferably 0.1 to 50 parts by mass with respect to 100 parts by mass of the resin in the resin composition. The lower limit thereof is more preferably 0.3 parts by mass or more and still more preferably 0.5 parts by mass or more. The upper limit thereof is more preferably 30 parts by mass or less, still more preferably 20 parts by mass or less, even still more preferably 10 parts by mass or less, even further still more preferably 5 parts by mass or less, and particularly preferably 4 parts by mass or less.

One kind or two or more kinds of base generators can be used. In a case where two or more kinds thereof are used, the total amount is preferably within the above-described range.

### <Solvent>

The resin composition according to the embodiment of the present invention preferably contains a solvent.

As the solvent, any publicly known solvent can be used. The solvent is preferably an organic solvent. Examples of the organic solvent include compounds such as esters, ethers, ketones, cyclic hydrocarbons, sulfoxides, amides, ureas, and alcohols.

Suitable examples of the esters include ethyl acetate, n-butyl acetate, isobutyl acetate, hexyl acetate, amyl formate, isoamyl acetate, butyl propionate, isopropyl butyrate, ethyl butyrate, butyl butyrate, methyl lactate, ethyl lactate, γ-butyrolactone, ε-caprolactone, δ-valerolactone, γ-valerolactone, alkyl alkyloxyacetate (for example, methyl alkyloxyacetate, ethyl alkyloxyacetate, and butyl alkyloxyacetate (for example, methyl methoxyacetate, ethyl methoxyacetate, butyl methoxyacetate, methyl ethoxyacetate, and ethyl ethoxyacetate)), 3-alkyloxypropionic acid alkyl esters (for example, methyl 3-alkyloxypropionate, and ethyl 3-alkyloxypropionate (for example, methyl 3-methoxypropionate, ethyl 3-methoxypropionate, methyl 3-ethoxypropionate, and ethyl 3-ethoxypropionate)), 2-alkyloxypropionic acid alkyl esters (for example, methyl 2-alkyloxypropionate, ethyl 2-alkyloxypropionate, and propyl 2-alkyloxypropionate (for example, methyl 2-methoxypropionate, ethyl 2-methoxypropionate, propyl 2-methoxypropionate, methyl 2-ethoxypropionate, and ethyl 2-ethoxypropionate)), methyl 2-alkyloxy-2-methylpropionate and ethyl 2-alkyloxy-2-methylpropionate (for example, methyl 2-methoxy-2-methylpropionate and ethyl 2-ethoxy-2-methylpropionate), methyl pyruvate, ethyl pyruvate, propyl pyruvate, methyl acetoacetate, ethyl acetoacetate, methyl 2-oxobutanoate, ethyl 2-oxobutanoate, ethyl hexanoate, ethyl heptanoate, dimethyl malonate, and diethyl malonate.

Suitable examples of ethers include ethylene glycol dimethyl ether, diethylene glycol dimethyl ether, diethylene glycol diethyl ether, diethylene glycol ethyl methyl ether, diethylene glycol butyl methyl ether, triethylene glycol dimethyl ether, tetraethylene glycol dimethyl ether, tetrahydrofuran, ethylene glycol monomethyl ether, ethylene glycol monoethyl ether, methyl cellosolve acetate, ethyl cellosolve acetate, diethylene glycol monomethyl ether, diethylene glycol monoethyl ether, diethylene glycol monobutyl ether, propylene glycol monomethyl ether, propylene glycol dimethyl ether, propylene glycol monomethyl ether acetate, propylene glycol monoethyl ether acetate, ethylene glycol monobutyl ether, ethylene glycol monobutyl ether acetate, diethylene glycol ethyl methyl ether, propylene glycol monopropyl ether acetate, and dipropylene glycol dimethyl ether.

Suitable examples of the ketones include methyl ethyl ketone, cyclohexanone, cyclopentanone, 2-heptanone, 3-heptanone, 3-methylcyclohexanone, levoglucosenone, and dihydrolevoglucosenone.

Suitable examples of the cyclic hydrocarbon include aromatic hydrocarbons such as toluene, xylene, and anisole, and cyclic terpenes such as limonene.

Suitable examples of the sulfoxides include dimethyl sulfoxide.

Suitable examples of the amide include N-methyl-2-pyrrolidone, N-ethyl-2-pyrrolidone, N-cyclohexyl-2-pyrrolidone, N,N-dimethylacetamide, N,N-dimethylformamide, N,N-dimethylisobutylamide, 3-methoxy-N,N-dimethylpropionamide, 3-butoxy-N,N-dimethylpropionamide, N-formylmorpholine, and N-acetylmorpholine.

Suitable examples of the urea include N,N,N′,N′-tetramethylurea and 1,3-dimethyl-2-imidazolidinone.

Examples of the alcohols include methanol, ethanol, 1-propanol, 2-propanol, 1-butanol, 1-pentanol, 1-hexanol, benzyl alcohol, ethylene glycol monomethyl ether, 1-methoxy-2-propanol, 2-ethoxyethanol, diethylene glycol monoethyl ether, diethylene glycol monohexyl ether, triethylene glycol monomethyl ether, propylene glycol monoethyl ether, propylene glycol monomethyl ether, polyethylene glycol monomethyl ether, polypropylene glycol, tetraethylene glycol, ethylene glycol monobutyl ether, ethylene glycol monobenzyl ether, ethylene glycol monophenyl ether, methylphenyl carbinol, n-amyl alcohol, methylamyl alcohol, and diacetone alcohol.

From the viewpoint of improving the properties of a coated surface or the like, it is also preferable to mix two or more kinds of solvents.

In the present invention, the solvent is preferably one solvent selected from methyl 3-ethoxypropionate, ethyl 3-ethoxypropionate, ethyl cellosolve acetate, ethyl lactate, diethylene glycol dimethyl ether, butyl acetate, methyl 3-methoxypropionate, 2-heptanone, cyclohexanone, cyclopentanone, γ-butyrolactone, γ-valerolactone, 3-methoxy-N,N-dimethylpropionamide, toluene, dimethyl sulfoxide, ethyl carbitol acetate, butyl carbitol acetate, N-methyl-2-pyrrolidone, propylene glycol methyl ether, and propylene glycol methyl ether acetate, as well as levoglucosenone and dihydrolevoglucosenone, or a mixed solvent composed of two or more of these. A combined use of dimethyl sulfoxide and γ-butyrolactone, a combined use of dimethyl sulfoxide and γ-valerolactone, a combined use of 3-methoxy-N,N-dimethylpropionamide and γ-butyrolactone, a combined use of 3-methoxy-N,N-dimethylpropionamide and dimethyl sulfoxide, or, a combined use of N-methyl-2-pyrrolidone and ethyl lactate is particularly preferable. An aspect in which an amount of about 1% to 10% by mass of toluene with respect to the total mass of the solvent is further added to these combinedly used solvents is also one of the preferred aspects of the present invention.

In particular, from the viewpoint of the storage stability or the like of the resin composition, an aspect in which γ-valerolactone is included as the solvent is also one of the preferred aspects of the present invention. In such an aspect, the content of γ-valerolactone with respect to the total mass of the solvent is preferably 50% by mass or more, more preferably 60% by mass or more, and still more preferably 70% by mass or more. In addition, the upper limit of the above-described content is not particularly limited, and it may be 100% by mass. The above-described content may be determined in consideration of the solubility or the like of the component contained in the resin composition, such as the specific resin.

In addition, in a case where dimethyl sulfoxide and γ-valerolactone are used in combination, with respect to the total mass of the solvent, it is preferable to contain 60% to 90% by mass of γ-valerolactone and 10% to 40% by mass of dimethyl sulfoxide, it is more preferable to contain 70% to 90% by mass of γ-valerolactone and 10% to 30% by mass of dimethyl sulfoxide, and it is still more preferable to contain 75% to 85% by mass of γ-valerolactone and 15% to 25% by mass of dimethyl sulfoxide.

Regarding the content of the solvent, the amount of the solvent is such that, from the viewpoint of coatability, the concentration of the total solid contents of the resin composition according to the embodiment of the present invention is preferably 5% to 80% by mass, more preferably 5% to 75% by mass, still more preferably 10% to 70% by mass, and even still more preferably 20% to 70% by mass. The content of the solvent may be adjusted depending on the desired thickness of the coating film and the coating method. In a case where two or more kinds of solvents are contained, the total thereof is preferably within the above-described range.

### <Metal Adhesiveness Improving Agent>

From the viewpoint of improving adhesiveness to a metal material used for an electrode, a wire, or the like, the resin composition according to the embodiment of the present invention preferably contains a metal adhesiveness improving agent. Examples of the metal adhesiveness improving agent include a silane coupling agent having an alkoxysilyl group, an aluminum-based auxiliary adhesive agent, a titanium-based auxiliary adhesive agent, a compound having a sulfonamide structure and a compound having a thiourea structure, a phosphoric acid derivative compound, a 0-ketoester compound, and an amino compound.

### [Silane Coupling Agent]

Examples of the silane coupling agent include the compounds described in paragraph 0316 of WO2021/112189A and the compounds described in paragraphs 0067 to 0078 of JP2018-173573A, the contents of which are incorporated in the present specification. In addition, it is also preferable to use two or more kinds of different silane coupling agents as described in paragraphs 0050 to 0058 of JP2011-128358A. It is also preferable to use the following compound as the silane coupling agent. In the following Formulae, Me represents a methyl group, and Et represents an ethyl group. In addition, examples of R shown below include a structure derived from a blocking agent in a blocked isocyanate group. The blocking agent may be selected depending on the desorption temperature; however, examples thereof include an alcohol compound, a phenol compound, a pyrazole compound, a triazole compound, a lactam compound, and an active methylene compound. It is preferably caprolactam or the like, for example, from the viewpoint of setting the desorption temperature to 160° C. to 180° C. Examples of the commercially available product of such a compound include X-12-1293 (manufactured by Shin-Etsu Chemical Co., Ltd.).

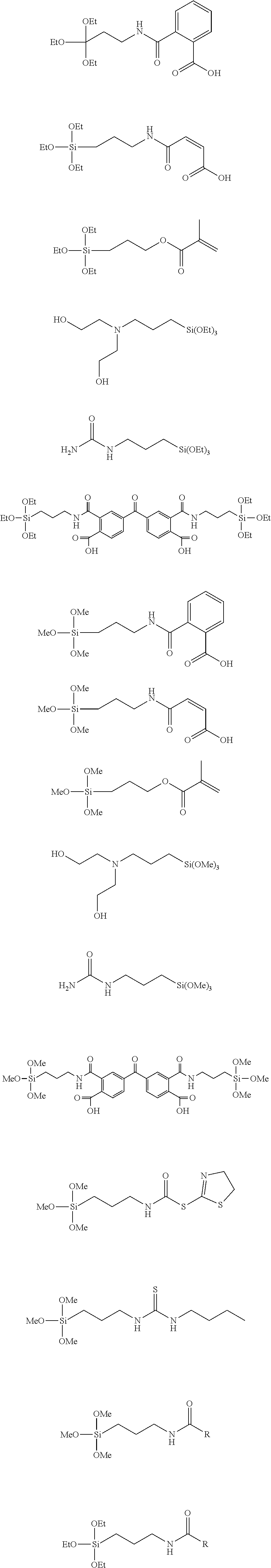

Examples of the other silane coupling agent include vinyltrimethoxysilane, vinyltriethoxysilane, 2-(3,4-epoxycyclohexyl)ethyltrimethoxysilane, 3-glycidoxypropylmethyldimethoxysilane, 3-glycidoxypropyltrimethoxysilane, 3-glycidoxypropylmethyldiethoxysilane, 3-glycidoxypropyltriethoxysilane, p-styryltrimethoxysilane, 3-methacryloxypropylmethyldimethoxysilane, 3-methacryloxypropyltrimethoxysilane, 3-methacryloxypropylmethyldiethoxysilane, 3-methacryloxypropyltriethoxysilane, 3-acryloxypropyltrimethoxysilane, N-2-(aminoethyl)-3-aminopropylmethyldimethoxysilane, N-2-(aminoethyl)-3-aminopropyltrimethoxysilane, 3-aminopropyltrimethoxysilane, 3-aminopropyltriethoxysilane, 3-triethoxysilyl-N-(1,3-dimethyl-butylidene)propylamine, N-phenyl-3-aminopropyltrimethoxysilane, tris-(trimethoxysilylpropyl)isocyanurate, 3-ureidopropyltrialkoxysilane, 3-mercaptopropylmethyldimethoxysilane, 3-mercaptopropyltrimethoxysilane, 3-isocyanatepropyltriethoxysilane, and 3-trimethoxysilylpropylsuccinic acid anhydride. These can be used alone or in a combination of two or more thereof.

In addition, an oligomer type compound having a plurality of alkoxysilyl groups can also be used as the silane coupling agent.

Examples of such an oligomer type compound include a compound containing a repeating unit represented by Formula (S-1).

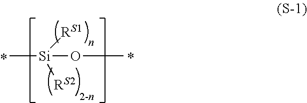

In Formula (S-1), R^{S1} represents a monovalent organic group, R^{S2} represents a hydrogen atom, a hydroxy group, or an alkoxy group, and n represents an integer of 0 to 2.

R^{S1} preferably has a structure including a polymerizable group. Examples of the polymerizable group include a group having an ethylenically unsaturated bond, an epoxy group, an oxetanyl group, a benzoxazolyl group, a blocked isocyanate group, and an amino group. Examples of the group having an ethylenically unsaturated bond include a vinyl group, an allyl group, an isoallyl group, a 2-methylallyl group, a group (for example, a vinylphenyl group) having an aromatic ring that is directly bonded to a vinyl group, and a (meth)acrylamide group, a (meth)acryloyloxy group, where a vinylphenyl group, a (meth)acrylamide group, or a (meth)acryloyloxy group is preferable, a vinylphenyl group or a (meth)acryloyloxy group is more preferable, and a (meth)acryloyloxy group is still more preferable.

R^{S2} is preferably an alkoxy group, and more preferably a methoxy group or an ethoxy group.

n represents an integer of 0 to 2, and it is preferably 1.

Here, structures of a plurality of repeating units represented by Formula (S-1) may be the same, where the plurality of repeating units are contained in the oligomer type compound.

Here, among a plurality of repeating units represented by Formula (S-1), which are contained in the oligomer type compound, it is preferable that at least one repeating unit has n of 1 or 2, it is more preferable that at least two repeating units have n of 1 or 2, and it is still more preferable that at least two repeating units have n of 1.

As such an oligomer type compound, a commercially available product can be used, and examples thereof include KR-513 (manufactured by Shin-Etsu Chemical Co., Ltd.).

### [Aluminum-Based Auxiliary Adhesive Agent]

Examples of the aluminum-based auxiliary adhesive agent include aluminum tris(ethyl acetoacetate), aluminum tris(acetyl acetate), and ethyl acetoacetate aluminum diisopropylate.

As another metal adhesiveness improving agent, the compounds described in paragraphs 0046 to 0049 of JP2014-186186A, and the sulfide-based compounds described in paragraphs 0032 to 0043 of JP2013-072935A can also be used, and the contents of which are incorporated in the present specification.

The content of the metal adhesiveness improving agent is preferably 0.01 to 30 parts by mass, more preferably 0.1 to 10 parts by mass, and still more preferably 0.5 to 5 parts by mass with respect to 100 parts by mass of the specific resin. In a case where the content is set to be equal to or higher than the above lower limit value, good adhesiveness between a pattern and a metal layer is exhibited, and in a case where the content is set to be equal to or lower than the above upper limit value, good heat resistance of the pattern and good mechanical characteristics are exhibited. Only one kind of metal adhesiveness improving agent may be used, or two or more kinds thereof may be used. In a case where two or more kinds thereof are used, the total content thereof is preferably within the above-described range.

### <Migration Suppressing Agent>

The resin composition according to the embodiment of the present invention preferably further contains a migration suppressing agent. For example, in a case where the resin composition is applied to a metal layer (or a metal wiring line) to form a film, it is possible to effectively suppress the migration of metal ions derived from the metal layer (or the metal wiring line) into the film, in a case where a migration suppressing agent is contained.

The migration suppressing agent is not particularly limited; however, examples thereof include a compound having a heterocyclic ring (a pyrrole ring, a furan ring, a thiophene ring, an imidazole ring, an oxazole ring, a thiazole ring, a pyrazole ring, an isoxazole ring, an isothiazole ring, a tetrazole ring, a pyridine ring, a pyridazine ring, a pyrimidine ring, a pyrazine ring, a piperidine ring, a piperazine ring, a morpholine ring, a 2H-pyran ring and a 6H-pyran ring, or a triazine ring), a compound having thioureas and a sulfanyl group, a hindered phenol-based compound, a salicylic acid derivative-based compound, and a hydrazide derivative-based compound. In particular, it is possible to preferably use a triazole-based compound such as 1,2,4-triazole, benzotriazole, 3-amino-1,2,4-triazole, or 3,5-diamino-1,2,4-triazole, or a tetrazole-based compound such as 1H-tetrazole, 5-phenyltetrazole or 5-amino-1H-tetrazole.

Among these, the resin composition according to the embodiment of the present invention preferably contains an azole compound.

The azole compound is a compound containing an azole structure, and the azole structure refers to a 5-membered ring structure including a nitrogen atom as a ring member, and is preferably a 5-membered ring structure including two or more nitrogen atoms as a ring member. Specific examples of the azole structure include an imidazole structure, a triazole structure, and a tetrazole structure. These structures may form a polycycle by fusion with another ring structure, such as benzimidazole or benzotriazole.

In addition, as the compound having an azole structure, a compound in which a group represented by Formula (R-1) or Formula (R-2) is directly bonded to the azole structure is also preferable.

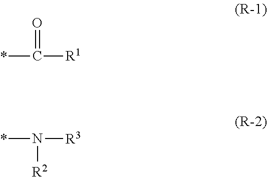

In Formula (R-1), R¹ represents a monovalent organic group, and * represents a bonding site to an azole structure.

In Formula (R-2), R² represents a hydrogen atom or a monovalent organic group, R³ represents a monovalent organic group, and * represents a bonding site to the azole structure.

In Formula (R-1), R¹ is preferably a hydrocarbon group or a group represented by a bond between a hydrocarbon group and at least one group selected from the group consisting of —O—, —C(═O)—, —S—, —S(═O)₂—, and —NR^{N}—. R^{N} is as described above.

The above-described hydrocarbon group is preferably an aliphatic hydrocarbon group, an aromatic hydrocarbon group, or a group represented by a combination thereof.

In addition, the total number of carbon atoms in R¹ is preferably 1 to 30, more preferably 2 to 25, and still more preferably 3 to 20.

In R¹, the bonding site to the carbonyl group in Formula (R-1) is preferably a hydrocarbon group or —NR^{N}—.

In Formula (R-1), * represents a bonding site to an azole structure, and is preferably a bonding site to a carbon atom which is a ring member of the azole structure.

In Formula (R-2), R² is preferably a hydrogen atom.

In a case where R² is a monovalent organic group, R² is preferably a hydrocarbon group or a group represented by a bond between a hydrocarbon group and at least one group selected from the group consisting of —O—, —C(═O)—, —S—, —S(═O)₂—, and —NR^{N}—. R^{N} is as described above.

The above-described hydrocarbon group is preferably an aliphatic hydrocarbon group, an aromatic hydrocarbon group, or a group represented by a combination thereof.

In addition, in a case where R² is a monovalent organic group, the total number of carbon atoms is preferably 1 to 30, more preferably 2 to 25, and still more preferably 3 to 20.

In a case where R² is a monovalent organic group, a bonding site to a nitrogen atom in Formula (R-2) in R² is preferably a hydrocarbon group or —C(═O)—.

In Formula (R-2), R³ is preferably a hydrocarbon group or a group represented by a bond between a hydrocarbon group and at least one group selected from the group consisting of —O—, —C(═O)—, —S—, —S(═O)₂—, and —NR^{N}—. R^{N} is as described above.

The above-described hydrocarbon group is preferably an aliphatic hydrocarbon group, an aromatic hydrocarbon group, or a group represented by a combination thereof.

In addition, in a case where R³ is a monovalent organic group, the total number of carbon atoms is preferably 1 to 30, more preferably 2 to 25, and still more preferably 3 to 20.

In R³, the bonding site to the nitrogen atom in Formula (R-2) is preferably a hydrocarbon group or —C(═O)—.

In Formula (R-2), * represents a bonding site to an azole structure, and is preferably a bonding site to a carbon atom which is a ring member of the azole structure.

As the migration suppressing agent, an ion trap agent that captures an anion such as a halogen ion can also be used.

The rust inhibitors described in paragraph 0094 of JP2013-015701A, the compounds described in paragraphs 0073 to 0076 of JP2009-283711A, the compounds described in paragraph 0052 of JP2011-059656A, the compounds described in paragraphs 0114, 0116, and 0118 of JP2012-194520A, the compounds described in paragraph 0166 of WO2015/199219A, or the like can be used as the other migration suppressing agents, the contents of which are incorporated in the present specification.

Specific examples of the migration suppressing agent include the following compounds.

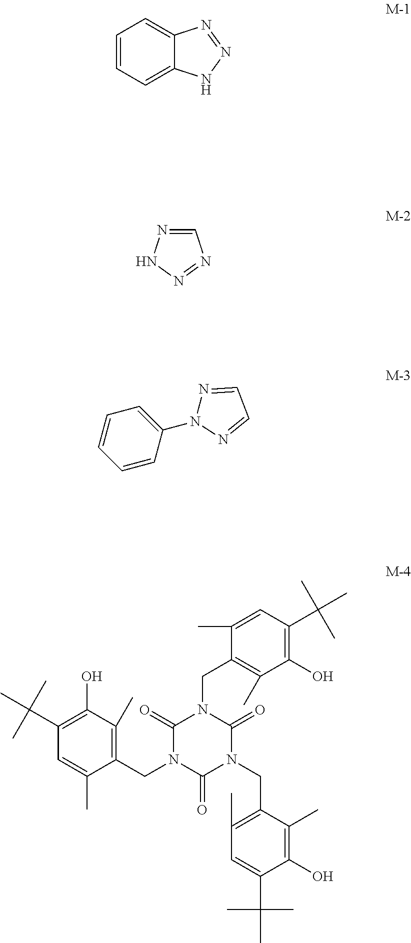 [SMILES: c1ccc2[nH]nnc2c1 ; c1nn[nH]n1 ; c1ccc(-n2nccn2)cc1 ; Cc1cc(C(C)(C)C)c(O)c(C)c1Cn1c(=O)n(Cc2c(C)cc(C(C)(C)C)c(O)c2C)c(=O)n(Cc2c(C)cc(C(C)(C)C)c(O)c2C)c1=O]

In a case where the resin composition according to the embodiment of the present invention contains the migration suppressing agent, the content of the migration suppressing agent is preferably 0.01% to 5.0% by mass, more preferably 0.05% to 2.0% by mass, and still more preferably 0.1% to 1.0% by mass, with respect to the total solid content of the resin composition.

Only one kind of migration suppressing agent may be used alone, or two or more kinds thereof may be used. In a case where two or more kinds of migration suppressing agents are used, the total thereof is preferably within the above-described range.

### <Light Absorbing Agent>

The resin composition according to the embodiment of the present invention also preferably contains a compound (a light absorbing agent) having a small absorbance at an exposure wavelength thereof upon exposure.

Examples of the light absorbing agent include compounds described in paragraphs 0159 to 0183 of WO2022/202647A and compounds described in paragraphs 0088 to 0108 of JP2019-206689A. The content thereof is incorporated in the present specification.

The content of the light absorbing agent with respect to the total solid content of the resin composition according to the embodiment of the present invention is not particularly limited, but is preferably 0.1% to 20% by mass, more preferably 0.5% to 10% by mass, and still more preferably 1% to 5% by mass.

### <Polymerization Inhibitor>

The resin composition according to the embodiment of the present invention preferably contains a polymerization inhibitor. Examples of the polymerization inhibitor include a phenol-based compound, a quinone-based compound, an amino-based compound, an N-oxyl-free radical-based compound, a nitro-based compound, a nitroso-based compound, a heteroaromatic ring-based compound, and a metal compound.

Specific examples of the compound of the polymerization inhibitor include the compounds described in paragraph 0310 of WO2021/112189A, p-hydroquinone, o-hydroquinone, a 4-hydroxy-2,2,6,6-tetramethylpiperidine-1-oxyl free radical, phenoxazine, and 1,4,4-trimethyl-2,3-diazabicyclo[3.2.2]nona-2-en-N,N-dioxide. The content thereof is incorporated in the present specification.

In a case where the resin composition according to the embodiment of the present invention has a polymerization inhibitor, the content of the polymerization inhibitor is preferably 0.01% to 20% by mass, more preferably 0.02% to 15% by mass, and still more preferably 0.05% to 10% by mass, with respect to the total solid content of the resin composition.

Only one kind of polymerization inhibitor may be used, or two or more kinds thereof may be used. In a case where two or more kinds of polymerization inhibitors are used, the total thereof is preferably within the above-described range.

### <Other Additives>

The resin composition according to the embodiment of the present invention may contain various additives as necessary, for example, a surfactant, a higher fatty acid derivative, a thermal polymerization initiator, inorganic particles, an ultraviolet absorbing agent, an organic titanium compounds, an antioxidant, a photoacid generator, an aggregation inhibitor, a phenol-based compound, another polymer compound, a plasticizer, and other auxiliary agents (for example, an anti-foaming agent, and a flame retardant) within the scope in which the effect of the present invention is obtained. By appropriately containing these components, properties such as film properties can be adjusted. The details of the components can be found in, for example, paragraph “0183” and subsequent paragraphs of JP2012-003225A (corresponding to paragraph 0237 of US2013/0034812A) and paragraphs 0101 to 0104 and 0107 to 0109 of JP2008-250074A, the contents of which are incorporated in the present specification. In a case where these additives are blended, the total content thereof is preferably 3% by mass or less of the solid content of the resin composition according to the embodiment of the present invention.

### [Inorganic Particle]

Specific examples of the inorganic particle include calcium carbonate, calcium phosphate, silica, kaolin, talc, titanium dioxide, alumina, barium sulfate, calcium fluoride, lithium fluoride, zeolite, molybdenum sulfide, and glass.

The average particle diameter of the inorganic particles is preferably 0.01 to 2.0 μm, more preferably 0.02 to 1.5 μm, still more preferably 0.03 to 1.0 μm, and particularly preferably 0.04 to 0.5 μm.

The average particle diameter of the inorganic particles is the primary particle diameter and the volume average particle diameter. The volume average particle diameter can be measured by, for example, a dynamic light scattering method with Nanotrac WAVE II EX-150 (manufactured by Nikkiso Co., Ltd.).

In a case where the above measurement is difficult, the measurement can also be carried out by a centrifugal sedimentation light transmission method, an X-ray transmission method, or a laser diffraction/light scattering method.

### [Organic Titanium Compound]

In a case where the resin composition contains an organic titanium compound, it is possible to form a resin layer having excellent chemical resistance even in a case where curing is carried out at a low temperature.

Examples of the usable organic titanium compound include those in which an organic group is bonded to a titanium atom through a covalent bond or an ionic bond.

Specific examples of the organic titanium compound are described in I) to VII) below.

I) Titanium chelate compounds: a titanium chelate compound having two or more alkoxy groups is more preferable since the resin composition has good storage stability and a good curing pattern is obtained. Specific examples thereof include titanium bis(triethanolamine)diisopropoxide, titanium di(n-butoxide) bis(2,4-pentanedionate), titanium diisopropoxide bis(2,4-pentanedionate), titanium diisopropoxide bis(tetramethylheptandionate), and titanium diisopropoxide bis(ethyl acetoacetate).

II) Tetraalkoxytitanium compounds: examples thereof include titanium tetra(n-butoxide), titanium tetraethoxide, titanium tetra(2-ethylhexoxide), titanium tetraisobutoxide, titanium tetraisopropoxide, titanium tetramethoxide, titanium tetramethoxypropoxide, titanium tetramethylphenoxide, titanium tetra(n-nonyloxide), titanium tetra(n-propoxide), titanium tetrastearyloxide, and titanium tetrakis[bis{2,2-(aryloxymethyl)butoxide}].

III) Titanocene compounds: examples thereof include pentamethylcyclopentadienyl titanium trimethoxide, bis(η5-2,4-cyclopentadien-1-yl)bis(2,6-difluorophenyl)titanium, and bis(η5-2,4-cyclopentadien-1-yl)bis(2,6-difluoro-3-(1H-pyrrol-1-yl)phenyl)titanium.

IV) Monoalkoxytitanium compounds: examples thereof include titanium tris(dioctyl phosphate)isopropoxide, and titanium tris(dodecylbenzene sulfonate)isopropoxide.

V) Titanium oxide compounds: examples thereof include titanium oxide bis(pentanedionate), titanium oxide bis(tetramethylheptandionate), and phthalocyanine titanium oxide.

VI) Titanium tetraacetylacetonate compounds: examples thereof include titanium tetraacetylacetonate.

VII) Titanate coupling agents: examples thereof include isopropyltridodecylbenzenesulfonyl titanate.

Among these, it is preferable that the organic titanium compound is at least one compound selected from the group consisting of the above-described I) titanium chelate compound, II) tetraalkoxytitanium compound, and III) titanocene compound, from the viewpoint of more favorable chemical resistance. In particular, titanium diisopropoxide bis(ethyl acetoacetate), titanium tetra(n-butoxide), or bis(η5-2,4-cyclopentadiene-1-yl) bis(2,6-difluoro-3-(1H-pyrrole-1-yl)phenyl)titanium is preferable.

In addition, it is also preferable that a compound represented by Formula (T-1) is contained as the organic titanium compound or instead of the organic titanium compound.

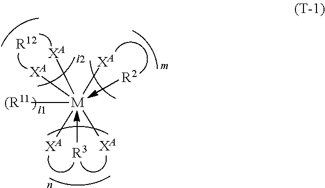

In Formula (T-1), M is titanium, zirconium, or hafnium, l1 is an integer of 0 to 2, l2 is 0 or 1, l1+l2×2 is an integer of 0 to 2, m is an integer of 0 to 4, n is an integer of 0 to 2, l1+l2+m+n×2=4 is satisfied, R¹¹'s are each independently a substituted or unsubstituted cyclopentadienyl group, a substituted or unsubstituted alkoxy group, or a substituted or unsubstituted phenoxy group, R¹² is a substituted or unsubstituted hydrocarbon group, R²'s are each independently a group including a structure represented by Formula (T-2), R³'s are each independently a group including a structure represented by Formula (T-2), and X^{A}'s are each independently an oxygen atom or a sulfur atom.

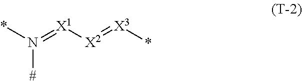

In Formula (T-2), X¹ to X³ each independently represent —C(—*)═ or —N═, *'s each represent a bonding site to another structure, and #represents a bonding site to a metal atom.

In Formula (T-1), it is preferable that M represents titanium from the viewpoint of the storage stability of the composition.

An aspect in which l1 and l2 in Formula (T-1) are 0 is also one of the preferred aspects of the present invention.

In Formula (T-1), m is preferably 2 or 4, and more preferably 2.

In Formula (T-1), n is preferably 1 or 2, and more preferably 1.

Here, in Formula (T-1), it is also preferable that l1 and l2 are 0 and m is 0, 2, or 4.

In Formula (T-1), R¹¹ is preferably a substituted or unsubstituted cyclopentadienyl ligand from the viewpoint of the stability of the specific metal complex.

In addition, the cyclopentadienyl group, the alkoxy group, and the phenoxy group as R¹¹ may be substituted; however, an aspect in which these are unsubstituted is also one of the preferred aspects of the present invention.

In Formula (T-1), R¹² is preferably a hydrocarbon group having 1 to 20 carbon atoms and more preferably a hydrocarbon group having 2 to 10 carbon atoms.

The hydrocarbon group as R¹² may be any of an aliphatic hydrocarbon group or an aromatic hydrocarbon group; however, it is preferably an aromatic hydrocarbon group.

The aliphatic hydrocarbon group may be a saturated aliphatic hydrocarbon group or an unsaturated aliphatic hydrocarbon group; however, it is preferably a saturated aliphatic hydrocarbon group.

The aromatic hydrocarbon group is preferably an aromatic hydrocarbon group having 6 to 20 carbon atoms, more preferably an aromatic hydrocarbon group having 6 to 10 carbon atoms, and still more preferably a phenylene group.

The substituent as R¹² is preferably a monovalent substituent, and examples thereof include a halogen atom. In addition, in a case where R¹² is an aromatic hydrocarbon group, it may have an alkyl group as a substituent.

Among these, in Formula (T-1), R¹² is preferably an unsubstituted phenylene group. In addition, the phenylene group as R¹² is preferably a 1,2-phenylene group.

In Formula (T-1), in a case where m is 2 or more and two or more R²'s contained, the structures of the two or more R²'s may be the same or different from each other.

In Formula (T-1), in a case where n is 2 or more and two or more R³'s contained, the structures of the two or more R³'s may be the same or different from each other.

In Formula (T-2), X¹ to X³ each independently represent —C(—*)═ or —N═. It is preferable that at least one thereof represents —C(—*)═, and it is more preferable that at least two thereof represent —C(—*)═.

Specific examples of the compound represented by Formula (T-1) include compounds corresponding to I-5 to I-8 in Examples, but the present invention is not limited thereto.

In a case of containing an organic titanium compound, the content thereof is preferably 0.05 to 10 parts by mass and more preferably 0.1 to 5 parts by mass with respect to 100 parts by mass of the specific resin. In a case where the content thereof is 0.05 parts by mass or more, the heat resistance and the chemical resistance of the cured pattern to be obtained are further improved, and in a case where the content thereof is 10 parts by mass or less, the storage stability of the composition is more excellent.

In a case of containing an organic titanium compound, the content thereof is preferably 0.05 to 10 parts by mass and more preferably 0.1 to 2 parts by mass with respect to 100 parts by mass of the specific resin. In a case where the content thereof is 0.05 parts by mass or more, the heat resistance and the chemical resistance of the cured pattern to be obtained are further improved, and in a case where the content thereof is 10 parts by mass or less, the storage stability of the composition is more excellent.

Examples of the other additives include the compounds described in paragraphs 0316 to 0358 of WO2022/145355A. The above description is incorporated in the present specification.

### <Characteristics of Resin Composition>

The viscosity of the resin composition according to the embodiment of the present invention can be adjusted by adjusting the concentration of solid contents of the resin composition. From the viewpoint of the coating film thickness, it is preferably 1,000 mm²/s to 12,000 mm²/s, more preferably 2,000 mm²/s to 10,000 mm²/s, and still more preferably 2,500 mm²/s to 8,000 mm²/s. Within the above range, it is easy to obtain a coating film having high uniformity. In a case of being 1,000 mm²/s or more, it is easy to carry out coating at a film thickness to be required as, for example, an insulating film for re-distribution of a wiring line, and in a case of being 12,000 mm²/s or less, a coating film having an excellent coating surface shape is obtained.

### <Restrictions on Substances Contained in Resin Composition>

The moisture content of the resin composition according to the embodiment of the present invention is preferably less than 2.0% by mass, more preferably less than 1.5% by mass, and still more preferably less than 1.0% by mass. In a case of being less than 2.0%, the storage stability of the resin composition is improved.

Examples of the method of maintaining the moisture content include adjusting the humidity under storage conditions and reducing the void ratio of the storage container during storage.

From the viewpoint of insulating properties, the metal content of the resin composition according to the embodiment of the present invention is preferably less than 5 parts per million (ppm) by mass, more preferably less than 1 ppm by mass, and still more preferably less than 0.5 ppm by mass. Examples of the metal include sodium, potassium, magnesium, calcium, iron, copper chromium, and nickel, however, a metal contained as a complex of an organic compound and a metal is excluded. In a case where a plurality of metals are contained, the total of these metals is preferably within the above-described range.

In addition, as a method of reducing metal impurities which are unintentionally contained in the resin composition according to the embodiment of the present invention, a method of selecting a raw material containing a low metal content as the raw material that constitutes the resin composition according to the embodiment of the present invention, a method of filtering a raw material constituting the resin composition according to the embodiment of the present invention, a method of distilling under the conditions in which the inside of the device is lined with polytetrafluoroethylene or the like to suppress the contamination as little as possible, and the like can be mentioned.

In the resin composition according to the embodiment of the present invention, in a case of considering the use application as a semiconductor material, the content of halogen atoms is preferably less than 500 ppm by mass, more preferably less than 300 ppm by mass, and still more preferably less than 200 ppm by mass, from the viewpoint of wire corrosiveness. Among these, in a case of being present in a halogen ion state, the content is preferably less than 5 ppm by mass, more preferably less than 1 ppm by mass, and still more preferably less than 0.5 ppm by mass. Examples of the halogen atom include a chlorine atom and a bromine atom. It is preferable that the total content of the chlorine atom and the bromine atom, or the total content of the chlorine ion and the bromine ion is within the above-described range.

Preferred examples of the method of adjusting the content of halogen atoms include ion exchange treatment.

A storage container publicly known in the related art can be used as a storage container for the resin composition according to the embodiment of the present invention. As the storage container, for the intended purpose of suppressing the incorporation of impurities into the raw materials and the resin composition according to the embodiment of the present invention, a multilayer bottle in which an inner wall of a container is composed of six kinds of six layers of resin, and a bottle with six kinds of resin being made as a seven-layer structure are preferably used. Examples of such a container include the container described in JP2015-123351A.

### <Cured Substance of Resin Composition>

In a case of curing the resin composition according to the embodiment of the present invention, it is possible to obtain a cured substance of the resin composition.

The cured substance according to the embodiment of the present invention is a cured substance formed by curing the resin composition.

The curing of the resin composition is preferably carried out by heating. The heating temperature is more preferably 120° C. to 400° C., still more preferably 140° C. to 380° C., and particularly preferably 170° C. to 350° C. The form of the cured substance of the resin composition is not particularly limited and can be selected depending on the use application, where the form includes a film shape, a rod shape, a spherical shape, a pellet shape, and the like. In the present invention, the cured substance preferably has a film shape. The shape of the cured substance can also be selected depending on the use application by the pattern processing of the resin composition, where the use application includes the formation of a protective film on a wall surface, formation of via holes for conduction, adjustment of impedance, electrostatic capacity, or internal stress, impartment of heat radiation function, and the like. The film thickness of the cured substance (the film consisting of the cured substance) is preferably 0.5 m or more and 150 m or less.

The shrinkage ratio of the resin composition according to the embodiment of the present invention after curing is preferably 50% or less, more preferably 45% or less, and still more preferably 40% or less. Here, the shrinkage ratio refers to a percentage of a change in the volume of the resin composition before and after curing, and it can be calculated according to the following expression.

$$Shrinkage ratio [ \% ] = 100 - ( volume after curing / volume before curing ) \times 100$$

### <Characteristics of Cured Substance of Resin Composition>

The imidization reaction rate of the cured substance of the resin composition according to the embodiment of the present invention is preferably 70% or more, more preferably 80% or more, and still more preferably 90% or more. In a case of being 70% or more, a cured substance having excellent mechanical properties may be obtained.

The breaking elongation of the cured substance of the resin composition according to the embodiment of the present invention is preferably 30% or more, more preferably 40% or more, and still more preferably 50% or more.

The glass transition temperature (Tg) of the cured substance of the resin composition according to the embodiment of the present invention is preferably 180° C. or higher, more preferably 210° C. or higher, and still more preferably 230° C. or higher.

### <Preparation of Resin Composition>

The resin composition according to the embodiment of the present invention can be prepared by mixing the above-described components. The mixing method is not particularly limited, and mixing can be carried out by methods publicly known in the related art.

Examples of the mixing method include mixing with a stirring blade, mixing with a ball mill, and mixing by rotating a tank.

The temperature during the mixing is preferably 10° C. to 30° C., and more preferably 15° C. to 25° C.

It is preferable to carry out filtration using a filter for the intended purpose of removing foreign substances such as dust and fine particles in the resin composition according to the embodiment of the present invention. The filter pore diameter is, for example, preferably 5 μm or less, more preferably 1 μm or less, still more preferably 0.5 μm or less, and even still more preferably 0.1 μm or less. The material of the filter is preferably polytetrafluoroethylene, polyethylene, or nylon. In a case where the material of the filter is polyethylene, it is more preferable to use high density polyethylene (HDPE). As the filter, a filter which has been washed with an organic solvent in advance may be used. In the filtration step using the filter, a plurality of kinds of filters may be connected in series or in parallel and used. In a case where a plurality of kinds of filters are used, filters having different pore diameters or different materials may be used in combination. Examples of the connection aspect include an aspect in which an HDPE filter having a pore diameter of 1 μm is connected in series as the first stage and an HDPE filter having a pore diameter of 0.2 μm is connected in series as the second stage. In addition, various materials may be filtered a plurality of times. In a case of being filtered a plurality of times, circulation filtration may be used. In addition, filtration may be carried out under pressure. In a case of carrying out pressurization to carry out filtration, the pressure for pressurization is, for example, preferably 0.01 MPa or more and 1.0 MPa or less, more preferably 0.03 MPa or more and 0.9 MPa or less, still more preferably 0.05 MPa or more and 0.7 MPa or less, and even still more preferably 0.05 MPa or more and 0.5 MPa or less.

In addition to filtration using a filter, impurity removal treatment using an adsorbing material may be carried out. The filtration using a filter and the impurity removal treatment using an adsorbing material may be combined. As the adsorbing material, a publicly known adsorbing material can be used. Examples thereof include an inorganic adsorbing material such as silica gel and zeolite and an organic adsorbing material such as activated carbon.

After filtration using a filter, a step of placing a bottle filled with the resin composition under reduced pressure to carry out degassing may be provided.

### (Manufacturing Method for Cured Substance)

The manufacturing method for a cured substance according to the embodiment of the present invention preferably includes a film forming step of applying a resin composition onto a base material to form a film.

The manufacturing method for a cured substance more preferably includes the above-described film forming step, an exposure step of selectively exposing the film formed by the film forming step, and a development step of developing the film exposed by the exposure step using a developer to form a pattern.

It is particularly preferable that the manufacturing method for a cured substance includes the film forming step, the exposure step, the development step, and at least one of a heating step of heating a pattern obtained by the development step or a post-development exposure step of exposing the pattern obtained by the development step.

In addition, it is also preferable that the manufacturing method for a cured substance includes the above-described film forming step and a step of heating the film.

Hereinafter, details of each step will be described.

### <Film Forming Step>

The resin composition according to the embodiment of the present invention can be applied onto a base material, thereby being used in a film forming step of forming a film.

The manufacturing method for a cured substance according to the embodiment of the present invention preferably includes a film forming step of applying a resin composition onto a base material to form a film.

### [Base Material]

The kind of the base material can be appropriately determined depending on the use application and is not particularly limited. Examples of the base material include a base material for semiconductor production, such as silicon, silicon nitride, polysilicon, silicon oxide, or amorphous silicon, quartz, glass, an optical film, a ceramic material, a vapor-deposited film, a magnetic film, a reflective film, a metal base material (for example, it may be any one of a base material formed from a metal or a base material having a metal layer formed by plating, vapor deposition, or the like) such as Ni, Cu, Cr, or Fe, paper, spin-on-glass (SOG), a thin film transistor (TFT) array base material, a mold base material, and an electrode plate of a plasma display panel (PDP). In particular, the base material is preferably a base material for semiconductor production, and more preferably a silicon base material, a Cu base material, or a mold base material.

A layer such as an intimate attachment layer made of hexamethyl disilazane (HMDS) or the like, or an oxide layer may be provided on the surface of these base materials.

The shape of the base material is not particularly limited, and it may be a circular shape or may be a rectangular shape.

In a case where the base material has a circular shape, the size of the base material is, for example, preferably a diameter of 100 to 450 mm and more preferably 200 to 450 mm. In a case of a rectangular shape, the length of the short side is, for example, preferably 100 to 1,000 mm and more preferably 200 to 700 mm.

As the base material, for example, a base material having a plate shape and preferably a base material (a substrate) having a panel shape are used.

In a case where the resin composition is applied to form a film on a surface of a resin layer (for example, a layer consisting of a cured substance) or on a surface of a metal layer, the resin layer or the metal layer serves as the base material.

The means for applying the resin composition onto a base material is preferably coating.

Specific examples of the means for application include a dip coating method, an air knife coating method, a curtain coating method, a wire bar coating method, a gravure coating method, an extrusion coating method, a spray coating method, a spin coating method, a slit coating method, and an ink jet method. From the viewpoint of the uniformity of the film thickness, a spin coating method, a slit coating method, a spray coating method, or an ink jet method is more preferable, and from the viewpoint of the uniformity of the film thickness and the viewpoint of productivity, a spin coating method or a slit coating method is more preferable. A film having a desired thickness can be obtained by adjusting the concentration of solid contents of the resin composition and application conditions according to the means to be applied. In addition, the coating method can be appropriately selected depending on the shape of the base material. In a case where a circular base material such as a wafer is used, a spin coating method, a spray coating method, an ink jet method, or the like is preferable, and in a case where a rectangular base material is used, a slit coating method, a spray coating method, an ink jet method, or the like is preferable. For example, the spin coating method can be applied at a rotation speed of 500 to 3,500 rpm for about 10 seconds to 3 minutes.

In addition, it is also possible to apply a method of transferring a coating film formed in advance on a temporary support by the above-described coating method, onto a base material.

Regarding the transfer method, the production methods described in paragraphs 0023 and 0036 to 0051 of JP2006-023696A and paragraphs 0096 to 0108 of JP2006-047592A can also be suitably used.

In addition, a step of removing an unnecessary film at the end part of the base material may be carried out. Examples of such a step include edge bead rinsing (EBR) and back rinsing.

A pre-wetting step of applying various solvents onto the base material before applying the resin composition onto the base material to improve the wettability of the base material and then applying the resin composition may be adopted.

### <Drying Step>

The above film may be subjected to a step (a drying step) of drying the film (or the layer) formed for removing the solvent, after the film forming step (the layer forming step).

That is, the manufacturing method for a cured substance according to the embodiment of the present invention may include a drying step of drying the film formed by the film forming step.

It is preferable that the drying step is carried out after the film forming step and before the exposure step.

The drying temperature of the film in the drying step is preferably 50° C. to 150° C., more preferably 70° C. to 130° C., and still more preferably 90° C. to 110° C. In addition, the drying may be carried out by reducing the pressure. Examples of the drying time include 30 seconds to 20 minutes, and the drying time is preferably 1 minute to 10 minutes and more preferably 2 minutes to 7 minutes.

### <Exposure Step>

The film may be subjected to an exposure step of selectively exposing the film.

The manufacturing method for a cured substance may include an exposure step of selectively exposing the film formed by the film forming step.

The selective exposure means that a part of the film is exposed. In addition, by selectively exposing the film, an exposed region (an exposed portion) and an unexposed region (a non-exposed portion) are formed in the film.

The exposure amount is not particularly limited as long as the resin composition according to the embodiment of the present invention can be cured; however, it is, for example, preferably 50 to 10,000 mJ/cm² and more preferably 200 to 8,000 mJ/cm² in terms of conversion of exposure energy at a wavelength of 365 nm.

The exposure wavelength can be appropriately determined in a range of 190 to 1,000 nm and preferably in a range of 240 to 550 nm.

Examples of the exposure wavelength, which are mentioned in the relationship with the light source, include (1) a semiconductor laser (wavelength: 830 nm, 532 nm, 488 nm, 405 nm, 375 nm, 355 nm, or the like); (2) a metal halide lamp; (3) a high pressure mercury lamp, a g-line (wavelength: 436 nm), an h-line (wavelength: 405 nm), an i-line (wavelength: 365 nm), or broadband light (three wavelengths of the g, h, and i-line); (4) an excimer laser, a KrF excimer laser (wavelength: 248 nm), an ArF excimer laser (wavelength: 193 nm), or an F₂ excimer laser (wavelength: 157 nm); (5) an extreme ultraviolet ray: EUV (wavelength: 13.6 nm); (6) an electron beam; and (7) a second harmonic wave of 532 nm and a third harmonic wave of 355 nm of a YAG laser. Regarding the resin composition according to the embodiment of the present invention, exposure with a high pressure mercury lamp is particularly preferable, and from the viewpoint of exposure sensitivity, exposure with an i-line is more preferable.

The exposure method is not particularly limited as long as at least a part of the film consisting of the resin composition according to the embodiment of the present invention is exposed; however, examples thereof include exposure using a photo mask and exposure by a laser direct imaging method.

### <Post-Exposure Heating Step>

The film may be subjected to a step of carrying out heating after the exposure (a post-exposure heating step).

That is, the manufacturing method for a cured substance according to the embodiment of the present invention may include a post-exposure heating step of heating the film exposed in the exposure step.

The post-exposure heating step can be carried out after the exposure step and before the development step.

The heating temperature in the post-exposure heating step is preferably 50° C. to 140° C. and more preferably 60° C. to 120° C.

The heating time in the post-exposure heating step is preferably 30 seconds to 300 minutes and more preferably 1 minute to 10 minutes.

In the post-exposure heating step, the temperature rising rate from the temperature at the start of heating to the maximum heating temperature is preferably 1 to 12° C./min, more preferably 2 to 10° C./min, and still more preferably 3 to 10° C./min.

In addition, the temperature rising rate may be appropriately changed during heating.

The heating means in the post-exposure heating step is not particularly limited, and a publicly known hot plate, oven, infrared heater, or the like can be used.

In addition, it is also preferable to carry out the heating in an atmosphere having a low oxygen concentration by allowing an inert gas such as nitrogen, helium, argon, or the like to flow.

### <Development Step>

The exposed film may be subjected to a development step of carrying out development using a developer to form a pattern.

That is, the manufacturing method for a cured substance according to the embodiment of the present invention may include a development step of developing the film exposed in the exposure step using a developer to form a pattern.

By carrying out the development, one of the exposed portion and the non-exposed portion of the film is removed, and a pattern is formed.

Here, the development in which the non-exposed portion of the film is removed by the development step is referred to as negative-tone development, and the development in which the exposed portion of the film is removed by the development step is referred to as positive-tone development.

### [Developer]

Examples of the developer that is used in the development step include a developer containing an alkaline aqueous solution or an organic solvent.

In a case where the developer is an alkaline aqueous solution, examples of the basic compound that can be contained in the alkaline aqueous solution include inorganic alkalis, primary amines, secondary amines, tertiary amines, and a quaternary ammonium salt. The basic compound is preferably tetramethylammonium hydroxide (TMAH), potassium hydroxide, sodium carbonate, sodium hydroxide, sodium silicate, sodium metasilicate, ammonia, ethylamine, n-propylamine, diethylamine, di-n-butylamine, triethylamine, methyldiethylamine, dimethylethanolamine, triethanolamine, tetraethylammonium hydroxide, tetrapropylammonium hydroxide, tetrabutylammonium hydroxide, tetrapentylammonium hydroxide, tetrahexylammonium hydroxide, tetraoctylammonium hydroxide, ethyltrimethylammonium hydroxide, butyltrimethylammonium hydroxide, methyltriamylammonium hydroxide, dibutyldipentylammonium hydroxide, dimethylbis(2-hydroxyethyl)ammonium hydroxide, trimethylphenylammonium hydroxide, trimethylbenzylammonium hydroxide, triethylbenzylammonium hydroxide, pyrrole, or piperidine, and it is more preferably TMAH. The content of the basic compound in the developer is preferably 0.01% to 10% by mass, more preferably 0.1% to 5% by mass, and still more preferably 0.3% to 3% by mass in the total mass of the developer.

In a case where the developer contains an organic solvent, the compounds described in paragraph 0387 of WO2021/112189A can be used as the organic solvent. The content thereof is incorporated in the present specification. In addition, suitable examples of the alcohols include methanol, ethanol, propanol, isopropanol, butanol, pentanol, octanol, diethylene glycol, propylene glycol, methyl isobutyl carbinol, and triethylene glycol, and suitable examples of the amides include N-methylpyrrolidone, N-ethylpyrrolidone, and dimethylformamide.

In a case where the developer contains an organic solvent, one kind of organic solvent can be used, or two or more kinds thereof can be mixedly used. In the present invention, in particular, a developer containing at least one selected from the group consisting of cyclopentanone, 7-butyrolactone, dimethyl sulfoxide, N-methyl-2-pyrrolidone, and cyclohexanone is preferable, a developer containing at least one selected from the group consisting of cyclopentanone, 7-butyrolactone, and dimethyl sulfoxide is more preferable, and a developer containing cyclopentanone is particularly preferable.

In a case where the developer contains an organic solvent, the content of the organic solvent with respect to the total mass of the developer is preferably 50% by mass or more, more preferably 70% by mass or more, still more preferably 80% by mass or more, and particularly preferably 90% by mass or more. In addition, the above content may be 100% by mass.

The developer may further contain another component.

Examples of the other component include a publicly known surfactant and a publicly known anti-foaming agent.

### [Method of Supplying Developer]

The method of supplying a developer is not particularly limited as long as a desired pattern can be formed, and it includes a method of immersing a base material on which a film has been formed in a developer, puddle development of supplying a developer to a film formed on a base material using a nozzle, and a method of continuously supplying a developer. The kind of nozzle is not particularly limited, and examples thereof include a straight nozzle, a shower nozzle, and a spray nozzle.

From the viewpoint of the permeability of the developer, the removability of the non-image area, and the manufacturing efficiency, a method of supplying a developer with a straight nozzle or a method of continuously supplying a developer with a spray nozzle is preferable, and from the viewpoint of the permeability of the developer into the image area, a method of supplying a developer with a spray nozzle is more preferable.

In addition, after the continuous supply by a straight nozzle, the base material is spun to remove the developer from the base material, and then the developer is continuously supplied by the straight nozzle again after the spin drying, a step of spinning the base material to remove the developer from the base material may be adopted, and this step may be repeated a plurality of times.

Examples of the method of supplying a developer in the development step include a step of continuously supplying a developer to a base material, a step of keeping a developer in a substantially stationary state on a base material, a step of vibrating a developer on a base material by ultrasonic waves or the like, and a step obtained by combining these steps.

The development time is preferably 10 seconds to 10 minutes and more preferably 20 seconds to 5 minutes. The temperature of the developer during development is not particularly determined; however, it is preferably 10° C. to 45° C. and more preferably 18° C. to 30° C.

In the development step, washing (rinsing) of the pattern with a rinsing liquid may be further carried out after the treatment with the developer. In addition, a method such as supplying a rinsing liquid before the developer which is in contact with the pattern is completely dried may be adopted.

### [Rinsing Liquid]

In a case where the developer is an alkaline aqueous solution, it is possible to use, for example, water as the rinsing liquid. In a case where the developer is a developer containing an organic solvent, it is possible to use as the rinsing liquid, for example, a solvent (for example, water, an organic solvent different from the organic solvent contained in the developer) different from the solvent contained in the developer.

Examples of the organic solvent in a case where the rinsing liquid contains an organic solvent include the same organic solvents as the organic solvents exemplified in the above-described case where the developer contains an organic solvent.

The organic solvent contained in the rinsing liquid is preferably an organic solvent different from the organic solvent contained in the developer, and it is more preferably an organic solvent having a solubility of the pattern, which is lower than that of the organic solvent contained in the developer.

In a case where the rinsing liquid contains an organic solvent, one kind of organic solvent can be used, or two or more kinds thereof can be mixedly used. The organic solvent is preferably cyclopentanone, γ-butyrolactone, dimethyl sulfoxide, N-methylpyrrolidone, cyclohexanone, PGMEA, or PGME, more preferably cyclopentanone, γ-butyrolactone, dimethyl sulfoxide, PGMEA, or PGME, and still more preferably cyclohexanone or PGMEA.

In a case where the rinsing liquid contains an organic solvent, the organic solvent is preferably 50% by mass or more, more preferably 70% by mass or more, and still more preferably 90% by mass or more with respect to the total mass of the rinsing liquid. In addition, the organic solvent may be 100% by mass with respect to the total mass of the rinsing liquid.

The rinsing liquid may further contain another component.

Examples of the other component include a publicly known surfactant and a publicly known anti-foaming agent.

### [Method of Supplying Rinsing Liquid]

The method of supplying a rinsing liquid is not particularly limited as long as a desired pattern can be formed and includes a method of immersing a base material in a rinsing liquid, a method of supplying a rinsing liquid to a base material by liquid filling, a method of supplying a rinsing liquid to a base material with a shower, and a method of continuously supplying a rinsing liquid to a base material by means such as a straight nozzle.

From the viewpoint of the permeability of the rinsing liquid, the removability of non-image area, and the manufacturing efficiency, there is a method of supplying a rinsing liquid with a shower nozzle, a straight nozzle, a spray nozzle, or the like, and a method of continuously supplying a rinsing liquid with a spray nozzle is preferable. From the viewpoint of the permeability of the rinsing liquid into the image area, a method of supplying a rinsing liquid with a spray nozzle is more preferable. The kind of nozzle is not particularly limited, and examples thereof include a straight nozzle, a shower nozzle, and a spray nozzle.

That is, the rinsing step is preferably a step of supplying, with a straight nozzle, or continuously supplying a rinsing liquid to the exposed film, and it is more preferably a step of supplying a rinsing liquid with a spray nozzle.

In the method of supplying a rinsing liquid in the rinsing step, a step of continuously supplying a rinsing liquid to a base material, a step of keeping a rinsing liquid in a substantially stationary state on a base material, a step of vibrating a rinsing liquid on the base material by ultrasonic waves or the like, and a step obtained by combining these steps can be adopted.

The rinsing time is preferably 10 seconds to 10 minutes and more preferably 20 seconds to 5 minutes. The temperature of the rinsing liquid during rinsing is not particularly determined; however, it is preferably 10° C. to 45° C. and more preferably 18° C. to 30° C.

### <Heating Step>

The pattern obtained by the development step (a pattern after the rinsing in a case where the rinsing step is carried out) may be subjected to a heating step of heating the pattern obtained by the development.

That is, the manufacturing method for a cured substance according to the embodiment of the present invention may include a heating step of heating the pattern obtained by the development step.

In addition, the manufacturing method for a cured substance according to the embodiment of the present invention may include a pattern obtained by another method without carrying out the development step, or a heating step of heating a film obtained by the film forming step.

In the heating step, the resin such as the polyimide precursor is cyclized to be a resin such as polyimide.

In addition, the crosslinking of unreacted crosslinkable groups in the specific resin or a polymerizable compound other than the specific resin also proceeds.

The heating temperature (the maximum heating temperature) in the heating step is preferably 50° C. to 450° C., more preferably 150° C. to 350° C., still more preferably 150° C. to 250° C., even still more preferably 160° C. to 250° C., and particularly preferably 160° C. to 230° C.

The heating step is preferably a step of accelerating the cyclization reaction of the polyimide precursor in the pattern under the action of the base or the like generated from the base generator by heating.

The heating in the heating step is preferably carried out at a temperature rising rate of 1 to 12° C./min from the temperature at the start of heating to the maximum heating temperature. The temperature rising rate is more preferably 2 to 10° C./min and still more preferably 3 to 10° C./min. In a case where the above temperature rising rate is set to 1° C./min or higher, the excessive volatilization of the acid or solvent can be prevented while securing productivity, and in a case where the above temperature rising rate is to 12° C./min or lower, the residual stress of the cured substance can be relaxed.

In addition, in a case of an oven that enables rapid heating, the heating is preferably carried out at a temperature rising rate of 1 to 8° C./sec from the temperature at the start of heating to the maximum heating temperature, more preferably 2 to 7° C./sec, and still more preferably 3 to 6° C./sec.

The temperature at the start of heating is preferably 20° C. to 150° C., more preferably 20° C. to 130° C., and still more preferably 25° C. to 120° C. The temperature at the start of heating refers to a temperature at which the step of heating to the maximum heating temperature is started. For example, in a case where the resin composition according to the embodiment of the present invention is applied on a base material and then dried, the temperature at the start of heating is the temperature of the film (the layer) after drying, and for example, it is preferable to raise the temperature from a temperature lower by 30° C. to 200° C. than the boiling point of the solvent contained in the resin composition.

The heating time (the heating time at the maximum heating temperature) is preferably 5 to 360 minutes, more preferably 10 to 300 minutes, and still more preferably 15 to 240 minutes.

In particular, in a case of forming a multilayered laminate, the heating temperature is preferably 30° C. or higher, more preferably 80° C. or higher, still more preferably 100° C. or higher, and particularly preferably 120° C. or higher, from the viewpoint of adhesiveness between layers.

The upper limit of the heating temperature is preferably 350° C. or lower, more preferably 250° C. or lower, and still more preferably 240° C. or lower.

The heating may be carried out stepwise. For example, a step in which the temperature is raised from 25° C. to 120° C. at 3° C./min, held at 120° C. for 60 minutes, raised from 120° C. to 180° C. at 2° C./min, and held at 180° C. for 120 minutes, may be carried out. In addition, it is also preferable to carry out the treatment while carrying out irradiation with ultraviolet rays as described in U.S. Pat. No. 9,159,547B. By such a pretreatment step, it is possible to improve the properties of the film. The pretreatment step may be carried out for a short time of about 10 seconds to 2 hours and more preferably 15 seconds to 30 minutes. The pretreatment step may be carried out as a step of two or more stages, for example, a first stage pretreatment step may be carried out in a range of 100° C. to 150° C., and then a second stage pretreatment step may be carried out in a range of 150° C. to 200° C.

Further, cooling may be carried out after heating, and the cooling rate, in this case, is preferably 1 to 5° C./min.

From the viewpoint of preventing the decomposition of the specific resin, it is preferable that the heating step is carried out in an atmosphere of a low oxygen concentration, for example, by allowing an inert gas such as nitrogen, helium, argon, or the like to flow, or carrying out heating under reduced pressure. The oxygen concentration is preferably 50 ppm (volume ratio) or lower, and more preferably 20 ppm (volume ratio) or lower.

The heating means in the heating step is not particularly limited; however, examples thereof include a hot plate, an infrared furnace, an electric heating oven, a hot air oven, and an infrared oven.

### <Post-Development Exposure Step>

The pattern obtained by the development step (a pattern after the rinsing in a case where the rinsing step is carried out) may be subjected to a post-development exposure step of exposing the pattern after the development step, instead of the heating step or in addition to the heating step.

That is, the manufacturing method for a cured substance according to the embodiment of the present invention may include a post-development exposure step of exposing the pattern obtained by the development step. The manufacturing method for a cured substance according to the embodiment of the present invention may include the heating step and the post-development exposure step or may include only one of the heating step and the post-development exposure step.

In the post-development exposure step, it is possible to accelerate, for example, a reaction in which the cyclization of a polyimide precursor or the like proceeds by photosensitization of a photobase generator, a reaction in which the elimination of an acid-decomposable group proceeds by photosensitization of a photoacid generator.

In the post-development exposure step, it is sufficient that at least a part of the pattern obtained in the development step is exposed; however, it is preferable that the whole of the above pattern is exposed.

The exposure amount in the post-development exposure step is preferably 50 to 20,000 mJ/cm² and more preferably 100 to 15,000 mJ/cm² in terms of conversion of exposure energy at the wavelength at which the photosensitive compound has a sensitivity.

The post-development exposure step can be carried out using, for example, the light source in the above-described exposure step, and it is preferable to use broadband light.

### <Metal Layer Forming Step>

The pattern (preferably a pattern that has been subjected to at least one of the heating step or the post-development exposure step) obtained by the development step may be subjected to a metal layer forming step of forming a metal layer on the pattern.

That is, it is preferable that the manufacturing method for a cured substance according to the embodiment of the present invention includes a metal layer forming step of forming a metal layer on the pattern (preferably a pattern that has been subjected to at least one of the heating step or the post-development exposure step) obtained by the development step.

For the metal layer, existing metal kinds can be used without particular limitations. Examples thereof include copper, aluminum, nickel, vanadium, titanium, chromium, cobalt, gold, tungsten, tin, silver, and an alloy including these metals, where copper or aluminum is more preferable, and copper is still more preferable.

The forming method for the metal layer is not particularly limited, and the existing method can be applied. For example, the methods described in JP2007-157879A, JP2001-521288A, JP2004-214501A, JP2004-101850A, U.S. Pat. No. 7,888,181B2, and U.S. Pat. No. 9,177,926B2 can be used. For example, photolithography, physical vapor deposition method (PVD), chemical vapor phase growth method (CVD), lift-off, electrolytic plating, electroless plating, etching, printing, and a method obtained by combining these may be conceivable. More specific examples of the forming method for the metal layer include a patterning method obtained by combining sputtering, photolithography, and etching, and a patterning method combining photolithography and electrolytic plating. Examples of the preferred aspect of the plating include electrolytic plating using a copper sulfate plating liquid or a copper cyanide plating liquid.

The thickness of the metal layer at the thickest portion is preferably 0.01 to 50 m and more preferably 1 to 10 m.

### <Use Application>

Examples of the field to which the manufacturing method for a cured substance according to the embodiment of the present invention or the cured substance according to the embodiment of the present invention can be applied include an insulating film of an electronic device, an interlayer insulating film for a re-distribution layer, and a stress buffer film. In addition, a sealing film, a substrate material (a base film or cover lay of a flexible print substrate, an interlayer insulating film), or such an insulating film in a use application for mounting as described above, which is patterned by etching, is mentioned. For these use applications, for example, Science & Technology Co., Ltd., “High functionality and applied technology of polyimide” April 2008, Technical library CMC TL “Basics and development of polyimide materials” supervised by Masaaki Kakimoto, published in November 2011, “Latest Polyimide Basics and Applications” edited by Japan Polyimide & Aromatic Polymers Study Group, NTS Inc., August 2010, or the like can be referred to.

The manufacturing method for a cured substance according to the embodiment of the present invention and the cured substance according to the embodiment of the present invention can also be used for the production of board surfaces such as an offset board surface or a screen board surface, for etching of molded parts, for the production of protective lacquers and dielectric layers in electronics, in particular, microelectronics.

### (Laminate and Manufacturing Method for Laminate)

A laminate according to the embodiment of the present invention refers to a structure body having a plurality of layers consisting of the cured substance according to the embodiment of the present invention.

The laminate is a laminate including two or more layers consisting of a cured substance, and it may be a laminate in which three or more layers are laminated.

At least one of the two or more layers consisting of a cured substance, which are included in the laminate, is a layer consisting of the cured substance according to the embodiment of the present invention, and from the viewpoint of suppressing the shrinkage of the cured substance, the deformation of the cured substance due to the shrinkage, or the like, it is also preferable that all the layers consisting of a cured substance which are included in the laminate are layers consisting of the cured substance according to the embodiment of the present invention.

That is, it is preferable that the manufacturing method for a laminate according to the embodiment of the present invention includes the manufacturing method for a cured substance according to the embodiment of the present invention, and it is more preferable to include repeating, a plurality of times, the manufacturing method for a cured substance according to the embodiment of the present invention.

In the laminate according to the embodiment of the present invention, an aspect in which two or more layers of layers consisting of a cured substance are included and a metal layer is provided between any of the layers consisting of the cured substance is preferable. The metal layer is preferably formed in the metal layer forming step.

That is, it is preferable that the manufacturing method for a laminate according to the embodiment of the present invention further includes a metal layer forming step of forming a metal layer on a layer consisting of the cured substance, between the manufacturing methods for a cured substance which are carried out a plurality of times. The preferred aspect of the metal layer forming step is as described above.

Examples of the preferred laminate include a laminate including at least a layer structure in which three layers of a layer consisting of a first cured substance, a metal layer, and a layer consisting of a second cured substance are laminated in order.

It is preferable that both the layer consisting of the first cured substance and the layer consisting of the second cured substance are layers consisting of the cured substance according to the embodiment of the present invention. The resin composition according to the embodiment of the present invention which is used for forming a layer consisting of the first cured substance and the resin composition according to the embodiment of the present invention which is used for forming a layer consisting of the second cured substance may have the same composition or may have compositions different from each other. The metal layer in the laminate according to the embodiment of the present invention is preferably used as the metal wiring line of the re-distribution layer or the like.

### <Laminating Step>

The manufacturing method for a laminate according to the embodiment of the present invention preferably further includes a laminating step.

The laminating step is a series of steps including carrying out again, in the following order on the surface of the pattern (the resin layer) or the metal layer, at least one of (a) the film forming step (the layer forming step), (b) the exposure step, (c) the development step, or (d) the heating step and the post-development exposure step. However, the aspect thereof may be such that at least one of (a) the film forming step or (d) the heating step and the post-development exposure step is repeated. In addition, (e) the metal layer forming step may be included after at least one of the heating step or the post-development exposure step of (d). It is needless to say that the laminating step may further include appropriately the above-described drying step or the like.

In a case where another laminating step is further carried out after the laminating step, a surface activation treatment step may be further carried out after the exposure step, the heating step, or the metal layer forming step. Examples of the surface activation treatment include plasma treatment. Details of the surface activation treatment will be described later.

The laminating step is preferably carried out 2 to 20 times and more preferably 2 to 9 times.

For example, a configuration having resin layers of 2 or more layers and 20 or fewer layers, such as a resin layer/a metal layer/a resin layer/a metal layer/a resin layer/a metal layer, is preferable, and a configuration having resin layers of 2 or more layers and 9 or fewer layers is still more preferable.

In the above layers, the compositions, shapes, film thicknesses, and the like may be the same or may be different from each other.

In the present invention, an aspect in which a metal layer is provided, and then furthermore, a cured substance (a resin layer) of the resin composition according to the embodiment of the present invention is formed to cover the metal layer is particularly preferable. Specific examples thereof include an aspect in which (a) the film forming step, (b) the exposure step, (c) the development step, (d) at least one of the heating step or the post-development exposure step, (e) the metal layer forming step are repeated in this order, and an aspect in which (a) the film forming step, (d) at least one of the heating step or the post-development exposure step, and (e) the metal layer forming step are repeated in order. By alternately carrying out the laminating step of laminating the resin composition layer (the resin layer) according to the embodiment of the present invention and the metal layer forming step, the resin composition layer (the resin layer) according to the embodiment of the present invention and the metal layer can be alternately laminated.

### (Surface Activation Treatment Step)

The manufacturing method for a laminate according to the embodiment of the present invention preferably includes a surface activation treatment step of subjecting at least a part of the metal layer or a part of the resin composition layer to a surface activation treatment.

The surface activation treatment step is usually carried out after the metal layer forming step (preferably, after at least one of the heating step or the post-development exposure step). However, after the development step, the metal layer forming step may be carried out after the resin composition layer is subjected to the surface activation treatment step.

Only at least a part of the metal layer may be subjected to the surface activation treatment, only at least a part of the resin composition layer after the exposure may be subjected to the surface activation treatment, or both at least a part of the metal layer and at least a part of the resin composition layer after the exposure may be subjected to the surface activation treatment. It is preferable to carry out the surface activation treatment on at least a part of the metal layer, and it is preferable to carry out the surface activation treatment on a part or whole of the region of the metal layer having a surface on which the resin composition layer is formed. In a case where a surface of the metal layer is subjected to the surface activation treatment in this manner, it is possible to improve the adhesiveness to the resin composition layer (film) to be provided on the surface thereof.

It is preferable that the surface activation treatment is carried out on a part or whole of the resin composition layer (the resin layer) after the exposure. In a case where a surface of the resin composition layer is subjected to the surface activation treatment in this manner, it is possible to improve the adhesiveness to a metal layer or a resin layer to be provided on the surface that has been subjected to the surface activation treatment. In particular, in a case where the resin composition layer is cured, such as in a case where negative-tone development is carried out, it is less likely to be damaged by the surface treatment, and thus the adhesiveness is likely to be improved.

The surface activation treatment can be carried out, for example, according to the method described in paragraph 0415 of WO2021/112189A. The content thereof is incorporated in the present specification.

### (Semiconductor Device and Manufacturing Method Therefor)

The present invention also discloses a semiconductor device, which includes the cured substance according to the embodiment of the present invention or the laminate.

In addition, the present invention also discloses a manufacturing method for a semiconductor device, which includes the manufacturing method for a cured substance according to the embodiment of the present invention or the manufacturing method for a laminate according to the embodiment of the present invention.

As the specific examples of the semiconductor device using the resin composition according to the embodiment of the present invention for forming an interlayer insulating film for a re-distribution layer, the description in paragraphs 0213 to 0218 and the description of FIG. 1 of JP2016-027357A can be referred to, the content of which is incorporated in the present specification.

### (Diamine Compound)

The diamine compound according to the embodiment of the present invention is a compound represented by Formula (4-1).

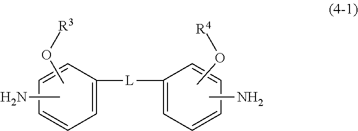

In Formula (4-1), R³ and R⁴ each independently represent a group having an ethylenically unsaturated bond, at least one of R³ or R⁴ has an aromatic hydrocarbon group, and L represents a single bond, —C(CH₃)₂—, or —C(CF₃)₂—.

In Formula (4-1), preferred aspects of R³ and R⁴ are the same as the preferred aspects of R¹ and R² in Formula (2-1) described above.

Among these, in Formula (4-1), it is preferable that R³ and R⁴ each independently have a vinylphenyl group.

The diamine according to the embodiment of the present invention can be obtained, for example, by reacting a diamine having a phenolic hydroxyl group represented by Formula (4-C) with a compound which contains a group having an ethylenically unsaturated bond and reacts with a phenolic hydroxyl group, such as benzyl halide substituted with a group having an ethylenically unsaturated bond or an isocyanate compound containing a group having an ethylenically unsaturated bond.

However, the synthesis method is not limited as long as the compound having the structure represented by Formula (4-1) is obtained.

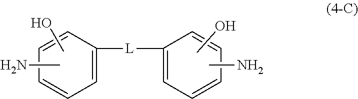

In Formula (4-C), L has the same definition as L in Formula (4-1) described above, and the same applies to the preferred aspect thereof.

### EXAMPLES

Hereinafter, the present invention will be described in detail using examples. Materials, using amounts, proportions, treatment details, treatment content, and the like shown in the following examples can be appropriately changed without departing from the gist of the present invention. Accordingly, the scope of the present invention is not limited to the following specific examples. Unless otherwise specified, “parts” and “%” are based on mass.

### (Synthesis of Monomer)

### Synthesis Example AA-1

375 mL of dimethylformamide was mixed with 48.65 g (225 mmol) of 3,3′-dihydroxybenzidine in a 1 L flask. 98.21 g (450 millimoles) of di-tert-butyl carbonate was added dropwise under ice cooling. Stirring was carried out at a temperature of 60° C. for 5 hours after completion of the dropwise addition. After the completion of the reaction, the mixture was cooled to room temperature, 35 mg of 2,2,6,6-tetramethylpiperidine 1-oxyl free radical, 68.68 g (450 mmol) of p-chloromethylstyrene, 74.63 g (540 mmol) of potassium carbonate, and 8.96 g (54.0 mmol) of potassium iodide were added thereto, and the mixture was stirred at a temperature of 60° C. for 3 hours. After completion of the reaction, filtration was carried out by a suction filtration operation, and the filtrate was added dropwise to 500 mL of water. Since white crystals were precipitated, the precipitated solid was collected by suction filtration. The obtained white solid was recrystallized and purified at 60° C. using 1000 mL of acetone. 125 g of the following intermediate AA-1a (yield: 85.6%) was obtained.

The structure of AA-1a is shown below. The following structure was confirmed from the ¹H-NMR spectrum.

¹H-NMR (BRUKER, AVANCE NEO 400): δ (ppm, DMSO-d6) 8.04-7.94 (s, 2H), 7.75-7.64 (d, 2H), 7.56-7.42 (m, 8H), 7.27-7.20 (d, 2H), 7.19-7.12 (d, 2H), 6.79-6.64 (2H), 5.89-5.77 (2H), 5.30-5.15 (6H), 1.49-1.43 (s, 18H)

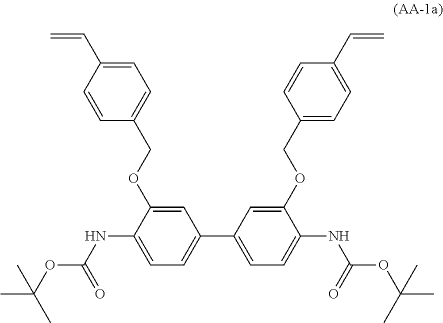 [SMILES: C=Cc1ccc(COc2cc(-c3ccc(NC(=O)OC(C)(C)C)c(OCc4ccc(C=C)cc4)c3)ccc2NC(=O)OC(C)(C)C)cc1]

75.0 g (115.6 mmol) of (AA-1a) and 500 mL of methylene chloride were mixed in a 1 L flask. 131.8 g (1156 mmol) of trifluoroacetic acid was added thereto at room temperature, and the mixture was stirred at a temperature of 40° C. for 5 hours. After completion of the reaction, 250 mL of methanol and then 117.0 g (1156 mmol) of triethylamine were added dropwise under ice cooling. Since pale yellow crystals were precipitated, the precipitated solid was collected by suction filtration. Suspension washing was carried out with 750 mL of methanol to obtain 40.5 g (yield: 73%) of (AA-1). The structure of AA-1 is shown below. The following structure was confirmed from the ¹H-NMR spectrum.

¹H-NMR (BRUKER, AVANCE NEO 400): δ (ppm, DMSO-d6) 7.53-7.45 (s, 8H), 7.05-6.98 (d, 2H), 6.92-6.85 (d, 2H), 6.79-6.63 (4H), 5.89-5.78 (d, 2H), 5.29-5.22 (d, 2H), 5.20-5.13 (s, 4H), 4.92-4.64 (4H)

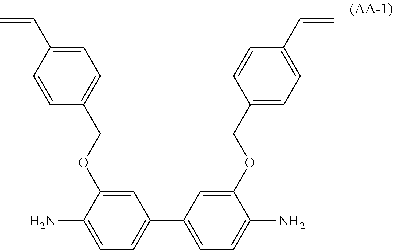 [SMILES: C=Cc1ccc(COc2cc(-c3ccc(N)c(OCc4ccc(C=C)cc4)c3)ccc2N)cc1]

### Synthesis Examples AA-2, AA-3, AA-4, AA-6, and AA-7

AA-2, AA-3, AA-4, AA-6, and AA-7 were obtained in the same method as in Synthesis Example AA-1, except that 3,3′-dihydroxybenzidine was appropriately changed. The structures of the respective compounds are shown below. The following structure was confirmed from the ¹H-NMR spectrum.

### Synthesis Example AA-5

AA-5 was obtained in the same method, except that in Synthesis Example AA-1, p-chloromethylstyrene was replaced with Karenz MOI (manufactured by Showa Denko K.K.). The structure of AA-5 is shown below. The following structure was confirmed from the ¹H-NMR spectrum.

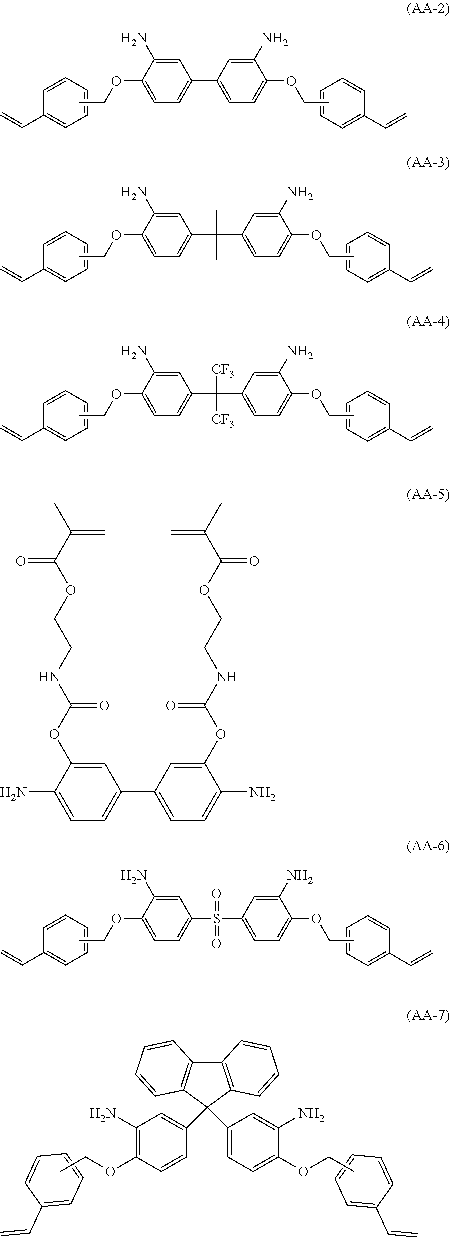

### Synthesis Example AT-1

AT-1 was obtained in the same method as in Synthesis Example AA-1, except that 3,3′-dihydroxybenzidine was replaced with p-methoxyphenol.

The structure of AT-1 is shown below. The following structure was confirmed from the ¹H-NMR spectrum.

¹H-NMR (BRUKER, AVANCE NEO 400): δ (ppm, DMSO-d6) 7.52-7.42 (d, 2H), 7.42-7.32 (d, 2H), 6.80-6.63 (3H), 6.54-6.43 (d, 2H), 5.92-5.77 (1H), 5.30-5.19 (1H), 4.95-4.89 (s, 2H), 4.68-4.54 (2H)

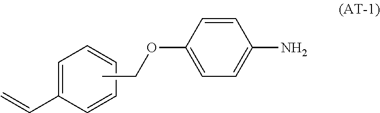

### (Synthesis of Polymer)

### Synthesis Example A-1: Synthesis of Polyimide (A-1)

30.0 g (57.64 mmol) of 4,4′-(4,4′-isopropylidenediphenoxy)diphtalic acid anhydride and 0.08 g of 2,2,6,6-tetramethylpiperidine 1-oxyl free radical were dissolved in 120 g of N-methylpyrrolidone (NMP) to obtain a solution. Subsequently, 9.93 g (24.2 mmol) of 2,2-bis[4-(4-aminophenoxy)phenyl]propane, 10.86 g (24.2 mmol) of AA-1, and 1.30 g (5.76 mmol) of AT-1 were dissolved in 100 g of NMP, and the solution was added dropwise to the above-described solution over 1 hour at a temperature of 0° C. to 10° C., and stirred at 25° C. for 60 minutes. Then, 18.2 g of pyridine and 14.7 g of acetic anhydride were added thereto, and the mixture was reacted at 80° C. for 4 hours. After completion of the reaction, the mixture was cooled to 25° C. and diluted with 200 g of tetrahydrofuran. Subsequently, the reaction solution was added dropwise to a mixed solution of 2.0 L of methanol and 0.5 L of water, and the mixture was stirred for 15 minutes and a polyimide resin was filtered. Next, the resin was subjected to reslurrying with 1 L of water, filtered, reslurried again with 1 L of methanol, filtered, and dried at 40° C. for 10 hours under reduced pressure. Subsequently, the resin dried as described above was dissolved in 250 g of tetrahydrofuran, 40 g of an ion exchange resin (MB-1: manufactured by ORGANO CORPORATION) was added thereto, the mixture was stirred for 4 hours, the ion exchange resin was filtered and removed, and then the polyimide resin was precipitated in 2 L of methanol and stirred for 15 minutes. The polyimide resin was filtered and acquired, and dried at 45° C. for 1 day under reduced pressure to obtain a polyimide resin (A-1). The obtained polyimide (A-1) had a weight-average molecular weight of 25,000 and a number-average molecular weight of 9,960. The polyimide (A-1) is a resin having a repeating unit represented by Formula (A-1). The structure of the repeating unit was determined from a ¹H-NMR spectrum. In the following structures, the subscripts of the repeating units represent the content molar ratio of the respective repeating units. The phenolic hydroxyl group value (content of phenolic hydroxyl group) of A-1 was 0.010 mmol/g, and ΔIm=2.1%.

The phenolic hydroxyl group value was calculated under the following measurement conditions.

### —Measurement Conditions—

¹H-NMR (BRUKER, AVANCE NEO 400)
Analysis software: TopSpin 4.0.7
Solvent: DMSO-d6
Internal standard: 1,3,5-trimethoxybenzene

In ¹H-NMR, a proton of a phenolic hydroxyl group was detected as a specific peak near δ (DMSO-d6)=10 ppm. In addition, the peak was detected at a specific peak at 6.1 ppm, which is the internal standard 1,3,5-trimethoxybenzene.

ΔIm is a rate of change in imide group value calculated by the following expression before and after heating at 350° C. and 1 atm for 1 hour.

$$Rate of change ( Δ Im , \% ) = ( Im 2 - Im 1 ) \times 10 \; 0 / Im 1$$

Im1: an imide group value before heating (mmol/g)
Im2: an imide group value (mmol/g) after heating at 350° C. and 1 atm for 1 hour

Specifically, the infrared absorption spectrum of the specific resin was measured, and a peak intensity P1 in the vicinity of 1,377 cm⁻¹, which is the absorption peak derived from the imide bond, was obtained. Next, the specific resin was subjected to a heat treatment at 350° C. and 1 atm for 1 hour, and then the infrared absorption spectrum was measured again to obtain a peak intensity P2 in the vicinity of 1,377 cm⁻¹. Using the obtained peak intensities P1 and P2, the rate of change in imide group value was determined based on the following expression. The peak intensity P1 is an index indicating an imide group value Im1 before heating, and the peak intensity P2 is an index indicating an imide group value Im2 after heating.

$$Ra te of change ( \% ) in imide group value = ( peak intensity P 2 - peak intensity P 1 ) \times 100 / peak intensity P 1$$

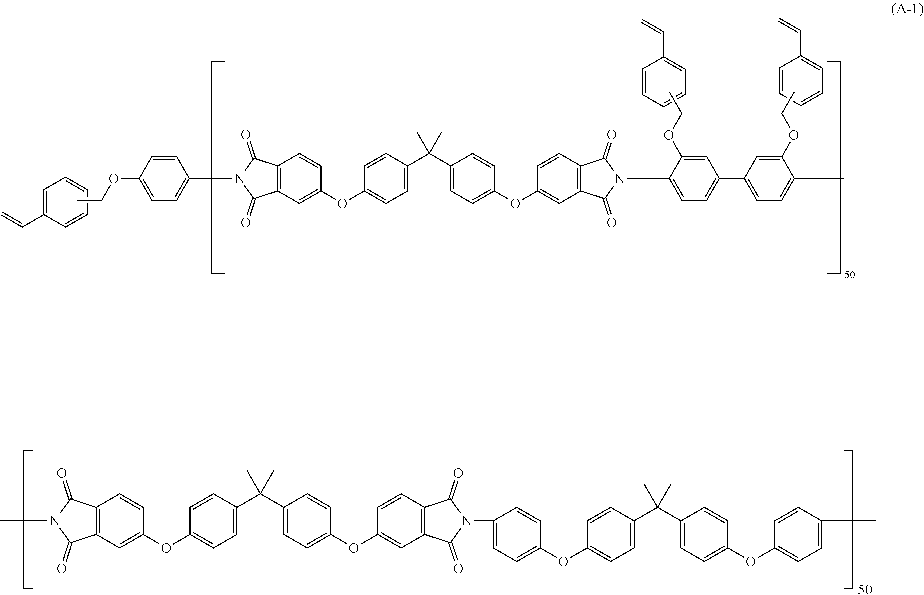

### Synthesis Example A-2: Synthesis of Polyimides (A-2 to 15)

Polyimides (A-2) to (A-15) were synthesized by the same method as that for the polyimide (A-1), except that the raw materials used were appropriately changed.

The polyimides (A-2) to (A-15) are resins having repeating units represented by Formulae (A-2) to (A-15), respectively. The structure of each repeating unit was determined from a ¹H-NMR spectrum. In the following structures, the description of the ratio indicates the content molar ratio of each structure. In addition, the weight-average molecular weight and the number-average molecular weight of these resins are shown in the following table.

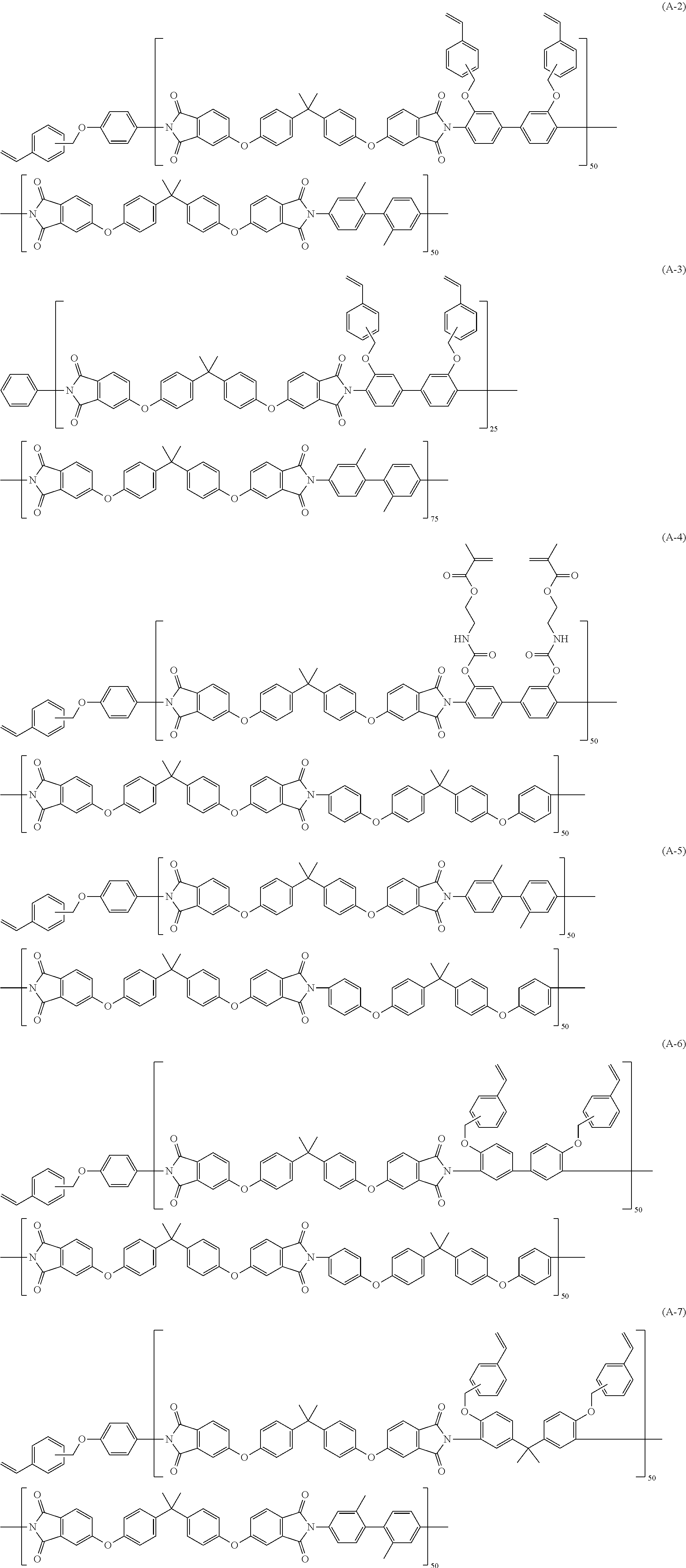

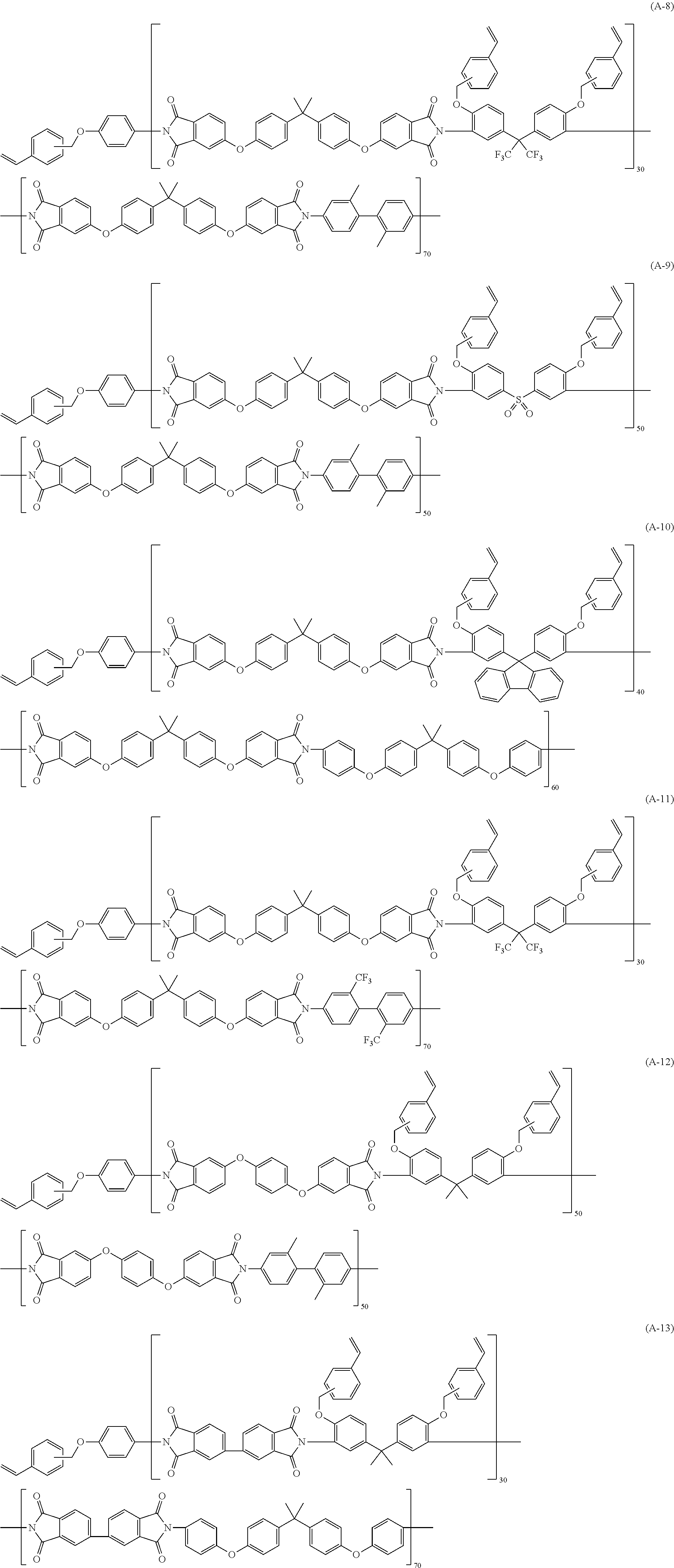

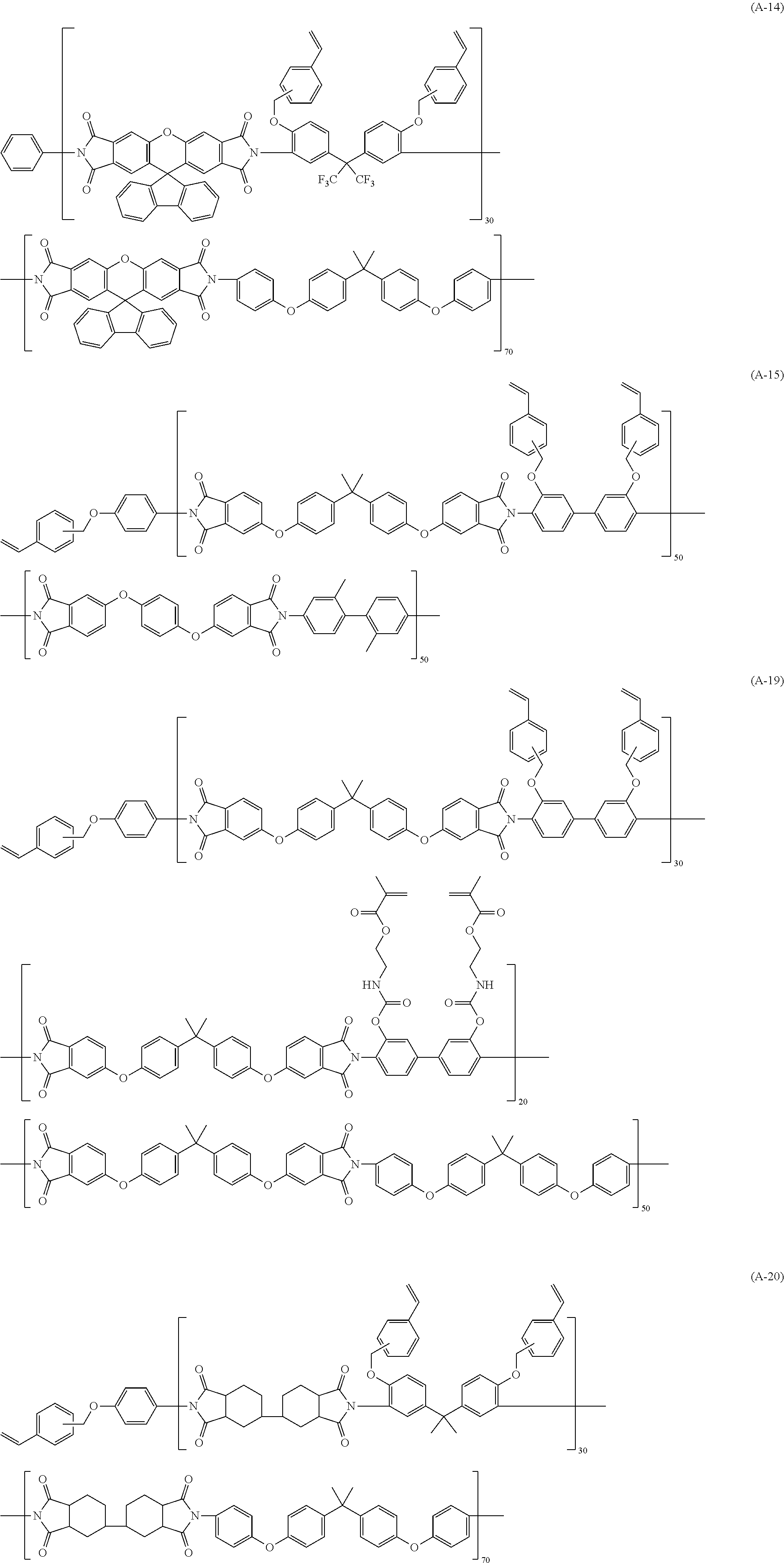

TABLE 1

| | | | Phenolic hydroxyl | |
|---|---|---|---|---|
| | Weight-average | Number-average | group value | ΔIm |
| Resin | molecular weight | molecular weight | (mmol/g) | (%) |
| A-2 | 20,000 | 8,060 | 0.015 | 1.0 |
| A-3 | 22,000 | 9,130 | 0.030 | 1.7 |
| A-4 | 21,000 | 8,800 | 0.040 | 1.5 |
| A-5 | 23,000 | 9,020 | 0.020 | 2.0 |
| A-6 | 21,000 | 8,940 | 0.075 | 2.0 |
| A-7 | 19,000 | 8,150 | 0.010 | 1.0 |
| A-8 | 22,500 | 9,180 | 0.015 | 1.5 |
| A-9 | 21,000 | 8,400 | 0.033 | 2.5 |
| A-10 | 28,500 | 10,900 | 0.080 | 2.8 |
| A-11 | 22,000 | 9,160 | 0.025 | 1.0 |
| A-12 | 19,500 | 8,230 | 0.015 | 1.5 |
| A-13 | 51,000 | 17,250 | 0.055 | 5.2 |
| A-14 | 19,000 | 7,450 | 0.105 | 1.3 |
| A-15 | 21,000 | 9,250 | 0.035 | 1.5 |
| A-19 | 24,500 | 10,100 | 0.040 | 1.5 |
| A-20 | 20,300 | 8,500 | 0.050 | 1.3 |

### Synthesis Example A-16: Synthesis of Polyimide (A-16)

A polyimide (A-16) was synthesized in the same method as the polyimide (A-1), except that in Synthesis Example 1, 10.86 g (24.2 mmol) of AA-1 was replaced with a mixture of 10.32 g (23.0 mmol) of AA-1 and 0.26 g (1.2 mmol) of 3,3′-dihydroxybenzidine. The obtained polyimide (A-16) had a weight-average molecular weight of 23,000 and a number-average molecular weight of 9,150.

The phenolic hydroxyl group value was 0.230 mmol/g, and ΔIm=2.0%.

### Synthesis Examples A-17 and A-18: Synthesis of Polyimides (A-17 and A-18)

Polyimides (A-17) and (A-18) were synthesized by the same method as that for the polyimide (A-1), except that 18.2 g of pyridine and 14.7 g of acetic anhydride were added in Synthesis Example 1, and the reaction was carried out at 80° C. for 4 hours, which was changed to 1 hour (A-17) or 2 hours (A-18). The obtained polyimide (A-17) had a weight-average molecular weight of 23,300, a number-average molecular weight of 9,250, a phenolic hydroxyl group value of 0.015 mmol/g, and ΔIm=25.5%. The polyimide (A-18) had a weight-average molecular weight of 24,700, a number-average molecular weight of 9,850, a phenolic hydroxyl group value of 0.010 mmol/g, and ΔIm=20.7%.

### Comparative Synthesis Example AC-1: Synthesis of Polyimide (AC-1)

24.74 g (40 mmol) of 4-[4-(1,3-dioxoisobenzofuran-5-ylcarbonyloxy)-2,3,5-trimethylphenyl]-2,3,6-trimethylphenyl 1,3-dioxoisobenzofuran-5-carboxylate was dissolved in 70 g of N-methylpyrrolidone (NMP) to obtain a solution. Subsequently, 6.45 g (17.6 mmol) of 2,2-bis(3-amino-4-hydroxyphenyl)hexafluoropropane and 5.64 g (17.6 mmol) of 4,4′-diamino-2,2′-bis(trifluoromethyl)biphenyl were dissolved in 50 g of NMP, and the solution was added dropwise to the above-described solution over 1 hour at a temperature of 10° C. to 25° C., stirred at 25° C. for 30 minutes, 10 g of toluene was added thereto, the mixture was reacted at 200° C. for 4 hours while nitrogen was flowing, and the mixture was cooled to 25° C. Subsequently, 4.97 g (35.2 mmol) of 2-isocyanatoethyl acrylate and 0.08 g of 2,2,6,6-tetramethylpiperidine 1-oxyl free radical were added thereto, the mixture was reacted at 95° C. for 15 hours, cooled to 25° C., and diluted with 120 g of tetrahydrofuran. Subsequently, the reaction solution was added dropwise to a mixed solution of 1.8 L of methanol and 0.6 L of water, and the mixture was stirred for 15 minutes and a polyimide resin was filtered. Next, the resin was subjected to reslurrying with 1 L of water, filtered, reslurried again with 1 L of methanol, filtered, and dried at 40° C. for 8 hours under reduced pressure. Subsequently, the resin dried as described above was dissolved in 250 g of tetrahydrofuran, 40 g of an ion exchange resin (MB-1: manufactured by ORGANO CORPORATION) was added thereto, the mixture was stirred for 4 hours, the ion exchange resin was filtered and removed, and then the polyimide resin was precipitated in 2 L of methanol and stirred for 15 minutes. The polyimide resin was filtered and acquired, and dried at 45° C. for 1 day under reduced pressure to obtain polyimide (AC-1). The polyimide (AC-1) is a resin having a repeating unit represented by Formula (AC-1). The structure of the repeating unit was determined from a ¹H-NMR spectrum. The obtained polyimide (AC-1) had a weight-average molecular weight of 19,500 and a number-average molecular weight of 7,450.

The phenolic hydroxyl group value was 0.370 mmol/g, and ΔIm=3.1%.

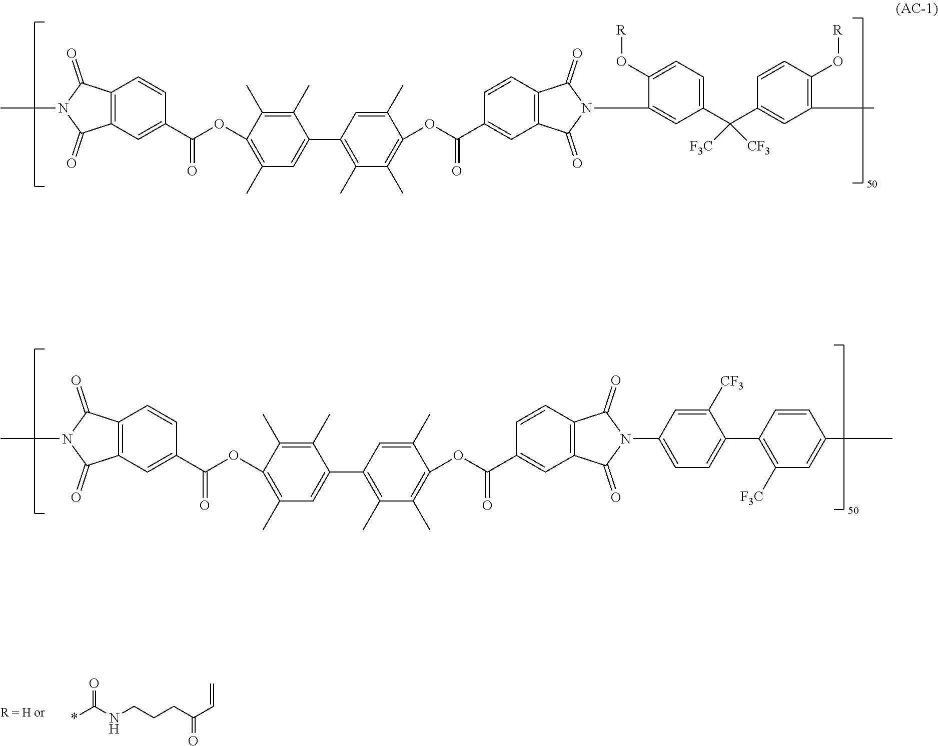

### Examples and Comparative Examples

In each of Examples, the components shown in the table were mixed to obtain each of the resin compositions. Further, in each Comparative Example, components shown in the table were mixed to obtain each of the comparative compositions.

Specifically, the content of each component described in the table other than the solvent was set to the amount (in terms of parts by mass) described in the column of “Parts by mass” of each column of the table.

The content of the solvent was set to an amount at which the concentration of solid contents of the composition was “Concentration of solid contents (% by mass)” in the table. In addition, the ratio of the amount of each solvent used was the content ratio (mass ratio) described in the column of “Ratio” in the table.

The obtained resin composition and the comparative composition were filtered under pressure using a filter made of polytetrafluoroethylene, having a micropore width of 0.8 m.

In addition, in the table, the description of “-” indicates that the corresponding component is not contained in the composition.

TABLE 2

| | | | | Comparative | Comparative | | | | | |
|---|---|---|---|---|---|---|---|---|---|---|
| | | | Example | Example | Example | Example | Example | Example | Example | Example |
| | | | 1 | 1 | 2 | 2 | 3 | 4 | 5 | 6 |
| Formulation | Resin | Mw | 25000 | 19500 | 20000 | 22000 | 25000 | 21000 | 20000 | 22000 |
| | | Type | A-1 | AC-1 | A-2 | A-5 | A-17 | A4 | A-2 | A-3 |
| | | Parts | 82.5 | 82.5 | 85 | 82.5 | 81.5 | 81.5 | 83 | 82.5 |
| | | by mass | | | | | | | | |
| | Polymerizable | Type | B-1 | B-1 | — | B-2 | B-1 | B-1 | B-1 | B-1 |
| | compound | Parts | 7.5 | 7.5 | — | 7.5 | 7.5 | 7.5 | 8.5 | 7.5 |
| | | by mass | | | | | | | | |
| | Polymerization | Type | C-1 | C-1 | C-1 | C-1 | C-2 | C-1 | C-3 | C-1 |
| | initiator | Pats | 3.5 | 3.5 | 6.5 | 3.5 | 3.5 | 3.5 | 3.5 | 3.5 |
| | | by mass | | | | | | | | |
| | Thermal-base | Type | D-1 | — | — | — | D-3 | D-2 | — | — |
| | generator | Parts | 1 | — | — | — | 1 | 1 | — | — |
| | | by mass | | | | | | | | |
| | Polymerization | Type | E-1 | E-1 | E-1 | E-2 | E-1 | E-1 | E-3 | E-1 |
| | inhibitor | Parts | 0.5 | 0.5 | 0.5 | 0.5 | 0.5 | 0.5 | 0.5 | 0.5 |
| | | by mass | | | | | | | | |
| | Silane coupling | Type | G-1 | G-1 | G1 | G-1 | G-1 | G-1 | G-2 | G-1 |
| | agent | Parts | 4.5 | 4.5 | 5.5 | 4.5 | 4.5 | 4.5 | 4.5 | 4.5 |
| | | by mass | | | | | | | | |
| | Migration | Type | F-1 | F-1 | F-2 | F-2 | F-3 | F-3 | — | F-1 |
| | suppressing | Parts | 0.5 | 0.5 | 0.5 | 0.5 | 0.5 | 0.5 | — | 0.5 |
| | agent | by mass | | | | | | | | |
| | Additive | Type | — | H-1 | H2 | H-1 | H-2 | H-1 | — | H-1 |
| | | Parts | — | 1 | 2 | 1 | 1 | 1 | — | 1 |
| | | by mass | | | | | | | | |
| | Titanium | Type | — | — | — | — | — | — | — | — |
| | compound | Parts | — | — | — | — | — | — | — | — |
| | | by mass | | | | | | | | |
| | Solvent | Type | GBL | GBL | GBL | GBL | GBL | GBL | GBL | GBL |
| | | Ratio | 80 | 80 | 80 | 80 | 80 | 80 | 80 | 80 |
| | | Type | DMSO | DMSO | DMSO | DMSO | DMSO | DMSO | DMSO | DMSO |
| | | Ratio | 20 | 20 | 20 | 20 | 20 | 20 | 20 | 20 |

| | | | | | | | | | |
|---|---|---|---|---|---|---|---|---|---|
| | Concentration of | 42 | 42 | 42 | 42 | 42 | 42 | 42 | 42 |
| | solid contents | | | | | | | | |
| | (% by mass) | | | | | | | | |
| Manufacture | Film thickness | 20 | 20 | 20 | 20 | 20 | 20 | 20 | 20 |
| | (μm) | | | | | | | | |
| | Developer | Cyclo- | Cyclo- | Cyclo- | Cyclo- | Cyclo- | Cyclo- | Cyclo- | Cyclo- |
| | | pentanone | pentanone | pentanone | pentanone | pentanone | pentanone | pentanone | pentanone |
| | Cure concentration | 230 | 230 | 230 | 230 | 230 | 230 | 230 | 230 |
| | (° C.) | | | | | | | | |
| | Cure time | 180 | 180 | 180 | 180 | 180 | 180 | 180 | 180 |
| | (min) | | | | | | | | |
| Evaluation | Df | A | C | C | B | B | B | A | A |
| | Reliability | A | C | C | B | B | A | B | A |
| | (post-HTS adhesion | | | | | | | | |
| | strength) | | | | | | | | |
| | Breaking elongation | A | C | C | B | B | B | A | A |
| | rate | | | | | | | | |

| | | | Example | Example | Example | Example | Example | Example | Example |
|---|---|---|---|---|---|---|---|---|---|
| | | | 7 | 8 | 9 | 10 | 11 | 12 | 13 |
| Formulation | Resin | Mw | 21000 | 23000 | 21000 | 19000 | 22500 | 21000 | 28500 |
| | | Type | A-4 | A-5 | A-6 | A-7 | A-8 | A-9 | A-10 |
| | | Parts | 82.5 | 82.5 | 82.5 | 82.5 | 82.5 | 82.5 | 82.5 |
| | | by mass | | | | | | | |
| | Polymerizable | Type | B-1 | B-2 | B-2 | B-1 | B-1 | B-1 | B-3 |
| | compound | Parts | 7.5 | 7.5 | 7.5 | 7.5 | 7.5 | 7.5 | 7.5 |
| | | by mass | | | | | | | |
| | Polymerization | Type | C-4 | C-1 | C-5 | C-1 | C-1 | C-1 | C-1 |
| | initiator | Pats | 3.5 | 3.5 | 3.5 | 3.5 | 3.5 | 3.5 | 3.5 |
| | | by mass | | | | | | | |
| | Thermal-base | Type | — | — | — | — | — | D-4 | — |
| | generator | Parts | — | — | — | — | — | 1 | — |
| | | by mass | | | | | | | |
| | Polymerization | Type | E-1 | E-1 | E-1 | E-1 | E-1 | E-1 | E-2 |
| | inhibitor | Parts | 0.5 | 0.5 | 0.5 | 0.5 | 0.5 | 0.5 | 0.5 |
| | | by mass | | | | | | | |
| | Silane coupling | Type | G-1 | G-3 | G-4 | G-1 | G-1 | G-1 | G-1 |
| | agent | Parts | 4.5 | 4.5 | 4.5 | 4.5 | 4.5 | 4.5 | 4.5 |
| | | by mass | | | | | | | |
| | Migration | Type | F-1 | F-1 | F-4 | F-5 | F-1 | F-1 | F-1 |
| | suppressing | Parts | 0.5 | 0.5 | 0.5 | 0.5 | 0.5 | 0.5 | 0.5 |
| | agent | by mass | | | | | | | |
| | Additive | Type | H-1 | H-1 | H-1 | H-1 | H-1 | — | H-1 |
| | | Parts | 1 | 1 | 1 | 1 | 1 | — | 1 |
| | | by mass | | | | | | | |
| | Titanium | Type | — | — | — | — | — | — | — |
| | compound | Parts | — | — | — | — | — | — | — |
| | | by mass | | | | | | | |
| | Solvent | Type | GBL | GBL | GBL | GBL | GBL | GBL | NMP |
| | | Ratio | 80 | 80 | 80 | 80 | 80 | 80 | 80 |
| | | Type | DMSO | DMSO | DMSO | DMSO | DMSO | DMSO | EL |
| | | Ratio | 20 | 20 | 20 | 20 | 20 | 20 | 20 |

| | | | | | | | | |
|---|---|---|---|---|---|---|---|---|
| | Concentration of | 42 | 42 | 42 | 42 | 42 | 42 | 42 |
| | solid contents | | | | | | | |
| | (% by mass) | | | | | | | |
| Manufacture | Film thickness | 20 | 20 | 20 | 20 | 20 | 20 | 20 |
| | (μm) | | | | | | | |
| | Developer | Cyclo- | Cyclo- | Cyclo- | Cyclo- | Cyclo- | Cyclo- | Cyclo- |
| | | pentanone | pentanone | pentanone | pentanone | pentanone | pentanone | pentanone |
| | Cure concentration | 230 | 230 | 230 | 230 | 230 | 230 | 230 |
| | (° C.) | | | | | | | |
| | Cure time | 180 | 180 | 180 | 180 | 180 | 180 | 180 |
| | (min) | | | | | | | |
| Evaluation | Df | A | A | A | A | A | A | A |
| | Reliability | A | A | A | A | A | A | A |
| | (post-HTS adhesion | | | | | | | |
| | strength) | | | | | | | |
| | Breaking elongation | A | A | A | A | A | A | A |
| | rate | | | | | | | |

TABLE 3

| | | | Example | Example | Example | Example | Example | Example | Example | Example |
|---|---|---|---|---|---|---|---|---|---|---|
| | | | 14 | 15 | 16 | 17 | 18 | 19 | 20 | 21 |
| Formulation | Resin | Mw | 22000 | 19500 | 51000 | 19000 | 21000 | 22000 | 22000 | 22500 |
| | | Type | A-11 | A-12 | A-13 | A-14 | A-15 | A-3 | A-3 | A-8 |
| | | Parts | 81.5 | 81.5 | 82.5 | 82.5 | 82.5 | 81.5 | 82.5 | 82.5 |
| | | by mass | | | | | | | | |
| | Polymerizable | Type | B-1 | B-1 | B-4 | B-2 | B-1 | B-1 | B-1 | B-1 |
| | compound | Parts | 7.5 | 7.5 | 7.5 | 7.5 | 7.5 | 6.5 | 7.5 | 7.5 |
| | | by mass | | | | | | | | |
| | Polymerization | Type | C-1 | C-6 | C-1 | C-1 | C-1 | C-7 | C-8 | C-1/C-9 |
| | initiator | Parts | 3.5 | 3.5 | 3.5 | 3.5 | 3.5 | 3.5 | 3.5 | 2.5/1.0 |
| | | by mass | | | | | | | | |
| | Thermal-base | Type | D-5 | D-6 | — | — | — | D-7 | — | — |
| | generator | Parts | 1 | 1 | — | — | — | 1 | — | — |
| | | by mass | | | | | | | | |
| | Polymerization | Type | E-1 | E-1 | E-1 | E-1 | E-1 | E-1 | E-1 | E-1 |
| | inhibitor | Parts | 0.5 | 0.5 | 0.5 | 0.5 | 0.5 | 0.5 | 0.5 | 0.5 |
| | | by mass | | | | | | | | |
| | Silane coupling | Type | G-1 | G-5 | G-1 | G-1 | G-1 | G-6 | G-1 | G-1 |
| | agent | Parts | 4.5 | 4.5 | 4.5 | 4.5 | 4.5 | 4.5 | 4.5 | 4.5 |
| | | by mass | | | | | | | | |
| | Migration | Type | F-6 | F-1 | F-7 | F-1 | F-1 | F-1 | F-1 | F-1 |
| | suppressing agent | Parts | 0.5 | 0.5 | 0.5 | 0.5 | 0.5 | 0.5 | 0.5 | 0.5 |
| | | by mass | | | | | | | | |
| | Additive | Type | H-1 | — | H-2 | H-1 | H-1 | H-1 | H-1 | H-1 |
| | | Parts | 1 | — | 1 | 1 | 1 | 1 | 1 | 1 |
| | | by mass | | | | | | | | |
| | Titanium | Type | — | I-1 | — | — | — | I-1 | — | — |
| | compound | Parts | — | 1 | — | — | — | 1 | — | — |
| | | by mass | | | | | | | | |
| | Solvent | Type | GBL | NMP | GBL | GBL | NMP | GBL | NMP | GBL |
| | | Ratio | 80 | 100 | 80 | 80 | 100 | 80 | 100 | 80 |
| | | Type | DMSO | — | DMSO | DMSO | — | DMSO | — | DMSO |
| | | Ratio | 20 | — | 20 | 20 | — | 20 | — | 20 |

| | | | | | | | | | |
|---|---|---|---|---|---|---|---|---|---|
| | Concentration of | 42 | 42 | 42 | 42 | 42 | 42 | 42 | 42 |
| | solid contents | | | | | | | | |
| | (% by mass) | | | | | | | | |
| Manufacture | Film thickness | 20 | 20 | 20 | 20 | 20 | 20 | 20 | 20 |
| | (μm) | | | | | | | | |
| | Developer | Cyclo- | Cyclo- | Cyclo- | Cyclo- | Cyclo- | Cyclo- | Cyclo- | Cyclo- |
| | | pentanone | pentanone | pentanone | pentanone | pentanone | pentanone | pentanone | pentanone |
| | Cure concentration | 230 | 230 | 230 | 230 | 230 | 230 | 230 | 230 |
| | (° C.) | | | | | | | | |
| | Cure time | 180 | 180 | 180 | 80 | 180 | 180 | 180 | 180 |
| | (min) | | | | | | | | |
| Evaluation | Df | A | A | A | A | A | A | A | A |
| | Reliability | A | A | A | A | A | A | A | A |
| | (post-HTS adhesion | | | | | | | | |
| | strength) | | | | | | | | |
| | Breaking elongation | A | A | A | A | A | A | A | A |
| | rate | | | | | | | | |

| | | | Example | Example | Example | Example | Example |
|---|---|---|---|---|---|---|---|
| | | | 22 | 23 | 24 | 25 | 26 |
| Formulation | Resin | Mw | 25000 | 22000 | 20000 | 25000 | 25000 |
| | | Type | A-1 | A-3 | A-2 | A-1 | A-1 |
| | | Parts | 82.5 | 82.5 | 82.5 | 82.5 | 82.5 |
| | | by mass | | | | | |
| | Polymerizable | Type | B-1 | B-1 | B-1 | B-1 | B-1 |
| | compound | Parts | 7.5 | 7.5 | 7.5 | 7.5 | 7.5 |
| | | by mass | | | | | |
| | Polymerization | Type | C-1/C-10 | C-2/C-11 | C-1 | C-1 | C-1 |
| | initiator | Parts | 2.0/1.5 | 1.0/2.5 | 3.5 | 3.5 | 3.5 |
| | | by mass | | | | | |
| | Thermal-base | Type | — | — | — | — | — |
| | generator | Parts | — | — | — | — | — |
| | | by mass | | | | | |
| | Polymerization | Type | E-1 | E-1 | E-4 | E-1 | E-5 |
| | inhibitor | Parts | 0.5 | 0.5 | 0.5 | 0.5 | 0.5 |
| | | by mass | | | | | |
| | Silane coupling | Type | G-1 | G-1 | G-1 | G-1 | G-1 |
| | agent | Parts | 4.5 | 4.5 | 4.5 | 4.5 | 4.5 |
| | | by mass | | | | | |
| | Migration | Type | F-8 | F-1 | F-1 | F-1 | F-1 |
| | suppressing agent | Parts | 0.5 | 0.5 | 0.5 | 0.5 | 0.5 |
| | | by mass | | | | | |
| | Additive | Type | H-1 | H-1 | H-1 | — | H-1 |
| | | Parts | 1 | 1 | 1 | — | 1 |
| | | by mass | | | | | |
| | Titanium | Type | — | — | — | I-2 | — |
| | compound | Parts | — | — | — | 1 | — |
| | | by mass | | | | | |
| | Solvent | Type | GBL | GBL | GBL | GBL | GBL |
| | | Ratio | 80 | 80 | 80 | 80 | 80 |
| | | Type | DMSO | DMSO | DMSO | DMSO | DMSO |
| | | Ratio | 20 | 20 | 20 | 20 | 20 |

| | | | | | | |
|---|---|---|---|---|---|---|
| | Concentration of | 42 | 42 | 42 | 42 | 42 |
| | solid contents | | | | | |
| | (% by mass) | | | | | |
| Manufacture | Film thickness | 20 | 20 | 20 | 20 | 20 |
| | (μm) | | | | | |
| | Developer | Cyclo- | Cyclo- | Cyclo- | Cyclo- | Cyclo- |
| | | pentanone | pentanone | pentanone | pentanone | pentanone |
| | Cure concentration | 230 | 230 | 230 | 230 | 230 |
| | (° C.) | | | | | |
| | Cure time | 180 | 180 | 180 | 180 | 180 |
| | (min) | | | | | |
| Evaluation | Df | A | A | A | A | A |
| | Reliability | A | A | A | A | A |
| | (post-HTS adhesion | | | | | |
| | strength) | | | | | |
| | Breaking elongation | A | A | A | A | A |
| | rate | | | | | |

TABLE 4

| | | | Example | Example | Example | Example | Example | Example | Example |
|---|---|---|---|---|---|---|---|---|---|
| | | | 27 | 28 | 29 | 30 | 31 | 32 | 33 |
| Formulation | Resin | Mw | 20000 | 22000 | 25000 | 20000 | 20000 | 25000 | 25000 |
| | | Type | A-2 | A-3 | A-1 | A-2 | A-2 | A-1 | A-1 |
| | | Parts | 82.5 | 81.5 | 81.5 | 82.5 | 82.5 | 82.5 | 83.5 |
| | | by mass | | | | | | | |
| | Polymerizable | Type | B-1 | B-1 | B-1 | B-1 | B-1 | B-1 | B-1 |
| | compound | Parts | 7.5 | 7.5 | 7.5 | 7.5 | 7.5 | 7.5 | 7.5 |
| | | by mass | | | | | | | |
| | Polymerization | Type | C-2 | C-1 | C-1 | C-2 | C-1 | C-1 | C-1 |
| | initiator | Parts | 3.5 | 3.5 | 3.5 | 3.5 | 3.5 | 3.5 | 3.5 |
| | | by mass | | | | | | | |
| | Thermal-base | Type | — | — | — | — | — | — | — |
| | generator | Parts | — | — | — | — | — | — | — |
| | | by mass | | | | | | | |
| | Polymerization | Type | E-1 | E-1 | E-4 | E-1 | E-1 | E-4 | E-2 |
| | inhibitor | Parts | 0.5 | 0.5 | 0.5 | 0.5 | 0.5 | 0.5 | 0.5 |
| | | by mass | | | | | | | |
| | Silane coupling | Type | G-1 | G-1 | G-1 | G-1 | G-1 | G-1 | G-1 |
| | agent | Parts | 4.5 | 4.5 | 4.5 | 4.5 | 4.5 | 4.5 | 4.5 |
| | | by mass | | | | | | | |
| | Migration | Type | F-1 | F-1 | F-1 | F-1 | F-1 | F-1 | F-1 |
| | suppressing agent | Parts | 0.5 | 0.5 | 0.5 | 0.5 | 0.5 | 0.5 | 0.5 |
| | | by mass | | | | | | | |
| | Additive | Type | H-3 | H-4 | H-5 | H-3 | — | — | — |
| | | Parts | 1 | 2 | 2 | 1 | — | — | — |
| | | by mass | | | | | | | |
| | Titanium | Type | — | — | — | — | I-3 | I-4 | — |
| | compound | Parts | — | — | — | — | 1 | 1 | — |
| | | by mass | | | | | | | |
| | Solvent | Type | NMP | NMP | GVL | NMP | GBL | NMP | MDMPA |
| | | Ratio | 80 | 80 | 80 | 100 | 100 | 80 | 30 |
| | | Type | CP | CH | DMSO | — | — | EL | GBL |
| | | Ratio | 20 | 20 | 20 | — | — | 20 | 70 |

| | | | | | | | | |
|---|---|---|---|---|---|---|---|---|
| | Concentration of | 42 | 42 | 42 | 42 | 42 | 42 | 42 |
| | solid contents | | | | | | | |
| | (% by mass) | | | | | | | |
| Manufacture | Film thickness | 20 | 20 | 20 | 20 | 20 | 20 | 20 |
| | (μm) | | | | | | | |
| | Developer | Cyclo- | Cyclo- | Cyclo- | Cyclo- | Cyclo- | Cyclo- | Cyclo- |
| | | pentanone | pentanone | pentanone | pentanone | pentanone | pentanone | pentanone |
| | Cure concentration | 230 | 230 | 230 | 180 | 180 | 180 | 230 |
| | (° C.) | | | | | | | |
| | Cure time | 180 | 180 | 180 | 120 | 120 | 120 | 180 |
| | (min) | | | | | | | |
| Evaluation | Df | A | A | A | A | A | A | A |
| | Reliability | A | A | A | A | A | A | A |
| | (post-HTS adhesion | | | | | | | |
| | strength) | | | | | | | |
| | Breaking elongation | A | A | A | A | A | A | A |
| | rate | | | | | | | |

| | | | Example | Example | Example | Example | Example | Example |
|---|---|---|---|---|---|---|---|---|
| | | | 34 | 35 | 36 | 37 | 38 | 39 |
| Formulation | Resin | Mw | 51000 | 51000 | 28500 | 25000/51000 | 25000 | 25000 |
| | | Type | A-4/A-13 | A-13 | A-10 | A-1/A-13 | A-1 | A-1 |
| | | Parts | 20/63.5 | 67.5 | 82.5 | 61.5/20.0 | 82.5 | 82.5 |
| | | by mass | | | | | | |
| | Polymerizable | Type | B-4 | B-4 | B-3 | B-1 | B-1 | B-2 |
| | compound | Parts | 6.5 | 15.5 | 7.5 | 8.5 | 7.5 | 7.5 |
| | | by mass | | | | | | |
| | Polymerization | Type | C-1 | C-1 | C-1 | C-1 | C-1 | C-1 |
| | initiator | Parts | 3.5 | 5.5 | 3.5 | 3.5 | 3.5 | 3.5 |
| | | by mass | | | | | | |
| | Thermal-base | Type | — | — | — | — | — | — |
| | generator | Parts | — | — | — | — | — | — |
| | | by mass | | | | | | |
| | Polymerization | Type | E-1 | E-1 | E-2 | E-1 | E-1 | E-1 |
| | inhibitor | Parts | 0.5 | 1.5 | 0.5 | 0.5 | 0.5 | 0.5 |
| | | by mass | | | | | | |
| | Silane coupling | Type | G-1 | G-1 | G-1 | G-1 | G-1 | G-1 |
| | agent | Parts | 4.5 | 6.5 | 4.5 | 4.5 | 4.5 | 4.5 |
| | | by mass | | | | | | |
| | Migration | Type | F-7 | F-7 | F-1 | F-1 | F-1 | F-1 |
| | suppressing agent | Parts | 0.5 | 1.5 | 0.5 | 0.5 | 0.5 | 0.5 |
| | | by mass | | | | | | |
| | Additive | Type | H-2 | H-2 | H-1 | H-1 | — | — |
| | | Parts | 1 | 2 | 1 | 1 | — | — |
| | | by mass | | | | | | |
| | Titanium | Type | — | — | — | — | I-5 | I-6 |
| | compound | Parts | — | — | — | — | 1 | 1 |
| | | by mass | | | | | | |
| | Solvent | Type | NMP | NMP | NMP | NMP | GBL | GBL |
| | | Ratio | 100 | 100 | 80 | 100 | 100 | 100 |
| | | Type | — | — | EL | — | — | — |
| | | Ratio | — | — | 20 | — | — | — |

| | | | | | | | |
|---|---|---|---|---|---|---|---|
| | Concentration of | 42 | 35 | 42 | 42 | 42 | 42 |
| | solid contents | | | | | | |
| | (% by mass) | | | | | | |
| Manufacture | Film thickness | 30 | 15 | 30 | 20 | 20 | 20 |
| | (μm) | | | | | | |
| | Developer | Cyclo- | Cyclo- | Cyclo- | Cyclo- | Cyclo- | Cyclo- |
| | | pentanone | pentanone | pentanone | pentanone | pentanone | pentanone |
| | Cure concentration | 230 | 230 | 180 | 230 | 230 | 230 |
| | (° C.) | | | | | | |
| | Cure time | 180 | 180 | 120 | 180 | 180 | 180 |
| | (min) | | | | | | |
| Evaluation | Df | A | A | A | A | A | A |
| | Reliability | A | A | A | A | A | A |
| | (post-HTS adhesion | | | | | | |
| | strength) | | | | | | |
| | Breaking elongation | A | A | A | A | A | A |
| | rate | | | | | | |

TABLE 5

| | | | Example | Example | Example | Example | Example | Example |
|---|---|---|---|---|---|---|---|---|
| | | | 40 | 41 | 42 | 43 | 44 | 45 |
| Formulation | Resin | Mw | 20000 | 20000 | 23000 | 24700 | 24500 | 20300 |
| | | Type | A-2 | A-2 | A-16 | A-18 | A-19 | A-20 |
| | | Parts | 82.5 | 82.5 | 82.5 | 82.5 | 82.5 | 82.5 |
| | | by mass | | | | | | |
| | Polymerizable | Type | B-1 | B-1 | B-1 | B-1 | B-1 | B-4 |
| | compound | Parts | 7.5 | 7.5 | 7.5 | 7.5 | 7.5 | 7.5 |
| | | by mass | | | | | | |
| | Polymerization | Type | C-1 | C-1 | C-1 | C-1 | C-8 | C-1 |
| | initiator | Parts | 3.5 | 3.5 | 3.5 | 3.5 | 3.5 | 3.5 |
| | | by mass | | | | | | |
| | Thermal-base | Type | — | — | — | — | — | — |
| | generator | Parts | — | — | — | — | — | — |
| | | by mass | | | | | | |
| | Polymerization | Type | E-1 | E-1 | E-1 | E-1 | E-1 | E-1 |
| | inhibitor | Parts | 0.5 | 0.5 | 0.5 | 0.5 | 0.5 | 0.5 |
| | | by mass | | | | | | |
| | Silane | Type | G-1 | G-1 | G-1 | G-1 | G-1 | G-1 |
| | coupling | Parts | 4.5 | 4.5 | 4.5 | 4.5 | 4.5 | 4.5 |
| | agent | by mass | | | | | | |
| | Migration | Type | F-1 | F-1 | F-1 | F-1 | F-1 | F-7 |
| | suppressing | Parts | 0.5 | 0.5 | 0.5 | 0.5 | 0.5 | 0.5 |
| | agent | by mass | | | | | | |
| | Additive | Type | — | — | — | — | H-1 | H-2 |
| | | Parts | — | — | — | — | 1 | 1 |
| | | by mass | | | | | | |
| | Titanium | Type | I-7 | I-8 | I-2 | I-2 | — | — |
| | compound | Parts | 1 | 1 | 1 | 1 | — | — |
| | | by mass | | | | | | |
| | Solvent | Type | NMP | NMP | GBL | GBL | NMP | GBL |
| | | Ratio | 100 | 100 | 80 | 80 | 100 | 100 |
| | | Type | — | — | DMSO | DMSO | — | — |
| | | Ratio | — | — | 20 | 20 | — | — |

| | | | | | | | |
|---|---|---|---|---|---|---|---|
| | Concentration of | 42 | 42 | 42 | 42 | 42 | 42 |
| | solid contents | | | | | | |
| | (% by mass) | | | | | | |
| Manufacture | Film thickness | 20 | 20 | 20 | 20 | 20 | 20 |
| | (μm) | | | | | | |
| | Developer | Cyclo- | Cyclo- | Cyclo- | Cyclo- | Cyclo- | Cyclo- |
| | | pentanone | pentanone | pentanone | pentanone | pentanone | pentanone |
| | Cure concentration | 230 | 230 | 230 | 230 | 230 | 230 |
| | (° C.) | | | | | | |
| | Cure time | 180 | 180 | 180 | 180 | 180 | 180 |
| | (min) | | | | | | |
| Evaluation | Df | A | A | B | A | A | A |
| | Reliability | A | A | B | B | A | A |
| | (post-HTS adhesion | | | | | | |
| | strength) | | | | | | |
| | Breaking elongation | A | A | A | A | A | A |
| | rate | | | | | | |

Details of each of the components listed in the table are as follows.

### [Resin]

A-1 to A-20, AC-1: polyimides A-1 to A-20, AC-1 synthesized above

A-1 to A-20 are compounds corresponding to the specific resin.

### [Polymerizable Compound]

B-1: SR-209 (manufactured by Sartomer Company Inc.)
B-2: ADPH: dipentaerythritol hexaacrylate (manufactured by Shin-Nakamura Chemical Co., Ltd.)
B-3: compound having the following structure
B-4: compound having the following structure

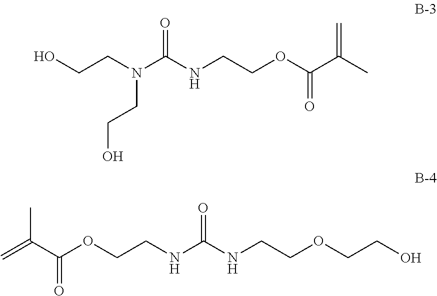

### [Polymerization Initiator]

C-1: IRGACURE OXE-01 (manufactured by BASF SE)
C-2: IRGACURE OXE-02 (manufactured by BASF SE)
C-3: IRGACURE 369 (manufactured by BASF SE)
C-4: compound having the following structure
C-5: compound having the following structure
C-6: compound having the following structure
C-7: Omnirad 1312 (manufactured by IGM Resins)
C-8: Omnirad TPO H (manufactured by IGM Resins)
C-9: CPI-310FG (manufactured by San-Apro Ltd.)
C-10: CPI-310B (manufactured by San-Apro Ltd.)
C-11: benzoyl peroxide (manufactured by Tokyo Chemical Industry Co., Ltd.)

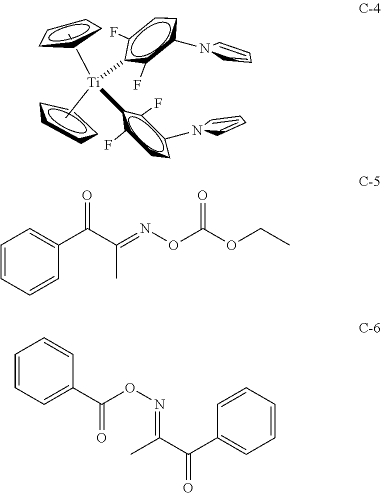

### [Thermal-Base Generator]

D-1 to D-7: compounds represented by Formulae (D-1) to (D-7)

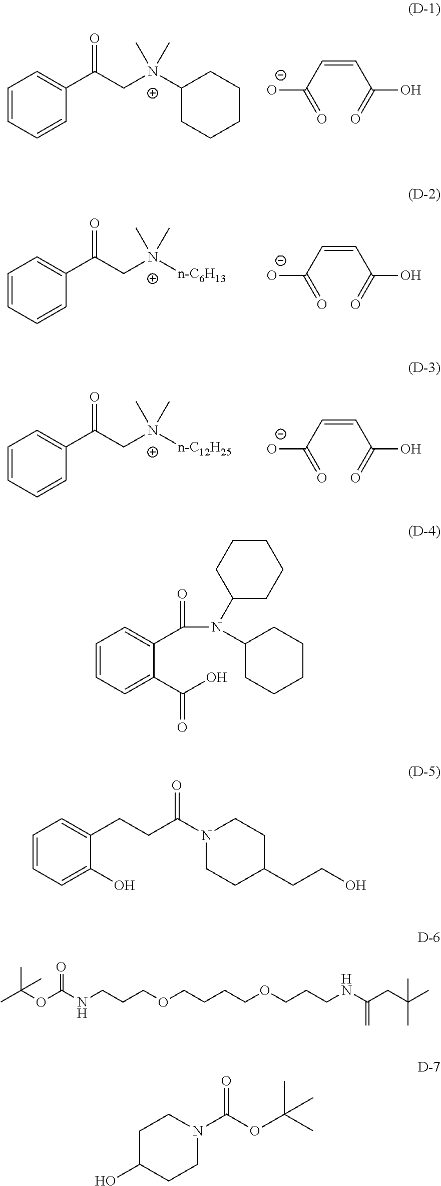

### [Polymerization Inhibitor]

E-1: 2-nitroso-1-naphthol (manufactured by Tokyo Chemical Industry Co., Ltd.)
E-2: parabenzoquinone (manufactured by Tokyo Chemical Industry Co., Ltd.)
E-3: paramethoxyphenol (manufactured by Tokyo Chemical Industry Co., Ltd.)
E-4: compound having the following structure
E-5: compound having the following structure

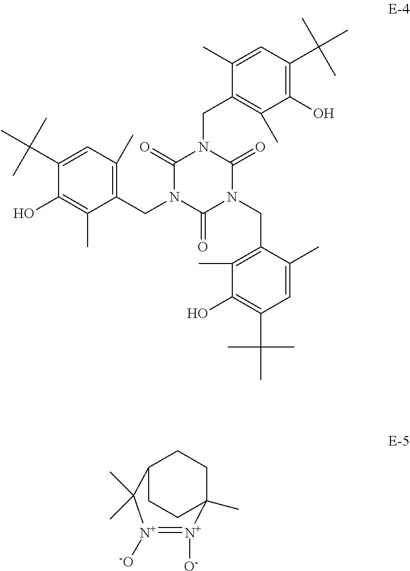

### [Migration Suppressing Agent]

F-1 to F-8: compounds having the following structures.

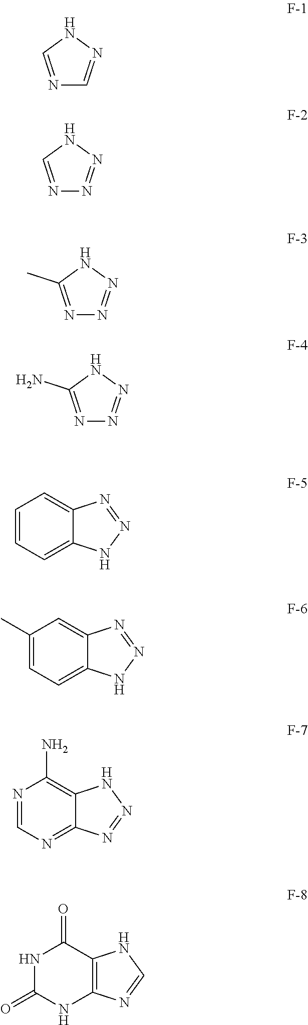

### [Silane Coupling Agent]

G-1 to G-4: compounds having the following structures. In the following structural formulae, Et represents an ethyl group.
G-5: X-12-1293 (manufactured by Shin-Etsu Chemical Co., Ltd.)
G-6: KR-513 (manufactured by Shin-Etsu Chemical Co., Ltd.)

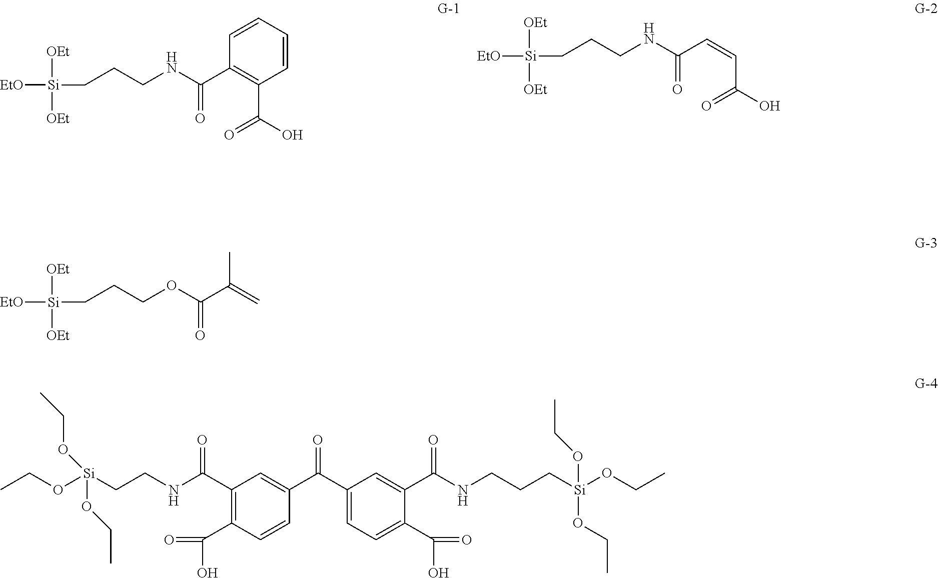 [SMILES: CCO[Si](CCCNC(=O)c1ccccc1C(=O)O)(OCC)OCC ; CCO[Si](CCCNC(=O)/C=C\C(=O)O)(OCC)OCC ; C=C(C)C(=O)OCCC[Si](OCC)(OCC)OCC ; CCO[Si](CCCNC(=O)c1cc(C(=O)c2ccc(C(=O)O)c(C(=O)NCC[Si](OCC)(OCC)OCC)c2)ccc1C(=O)O)(OCC)OCC]

### [Solvent]

GBL: γ-butyrolactone
DMSO: dimethyl sulfoxide
NMP: N-methyl-2-pyrrolidone
EL: ethyl lactate
GVL: γ-valerolactone
MDMPA: KJCMPA-100 (manufactured by KJ Chemicals Co., Ltd.)
CP: cyclopentanone
CH: cyclohexanone

### [Additive]

H-1: compound having the following structure
H-2: N-phenyldiethanolamine
H-3: compound having the following structure
H-4: compound having the following structure
H-5: compound having the following structure

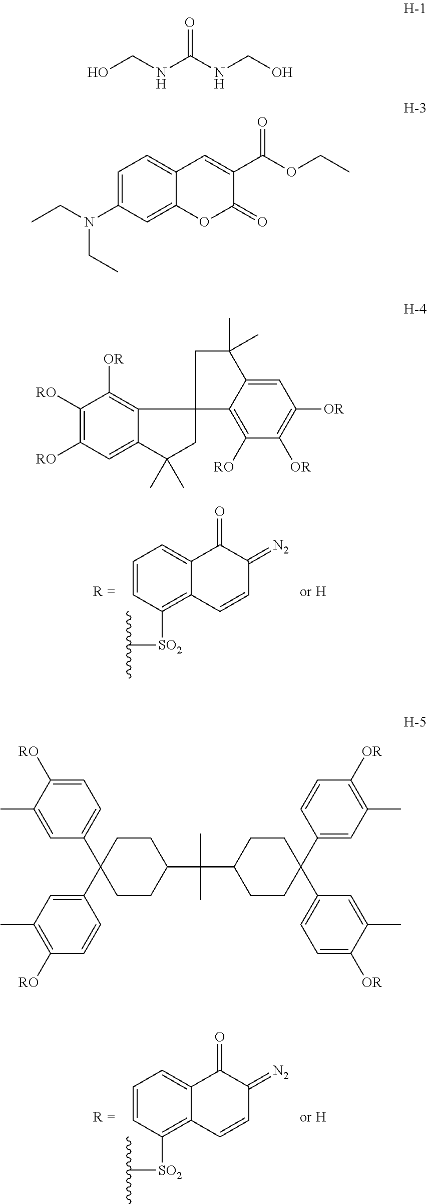

H-5 was synthesized based on the following synthesis method.

### [Synthesis Method of H-5]

29.72 g (70 mmol) of 4,4′-(1-(2-(4-hydroxyphenyl)-2-propyl)phenyl)ethylidene)bisphenol (manufactured by Honshu Chemical Industry Co., Ltd.: Tris-PA)) was added to a flask. Subsequently, 46.93 g (174.9 mmol) of 1,2-naphthoquinone diazide-5-sulfonic acid chloride and 17.9 g of triethylamine were dissolved in 300 g of acetone with stirring, the solution was added dropwise to the flask using a dropping funnel for 30 minutes, and the solution was stirred at an internal temperature of 30° C. for 30 minutes. Subsequently, hydrochloric acid was added dropwise thereto, and the mixture was further stirred for 30 minutes. Subsequently, a solution of 1640 g of pure water and 30 g of hydrochloric acid was prepared in a beaker, the filtrate obtained by filtering the hydrochloride in the reaction solution was added dropwise thereto, the precipitate was filtered, washed with water, and vacuum-dried at 40° C. for 50 hours to obtain a diazonaphthoquinone compound H-5.

### [Titanium Compound]

I-1: TC-750 (manufactured by Matsumoto Fine Chemical Co., Ltd.)
I-2: TC-401 (manufactured by Matsumoto Fine Chemical Co., Ltd.)
I-3: TC-800 (manufactured by Matsumoto Fine Chemical Co., Ltd.)
I-4: TC-810 (manufactured by Matsumoto Fine Chemical Co., Ltd.)
I-5 to I-8: compounds having the following structures

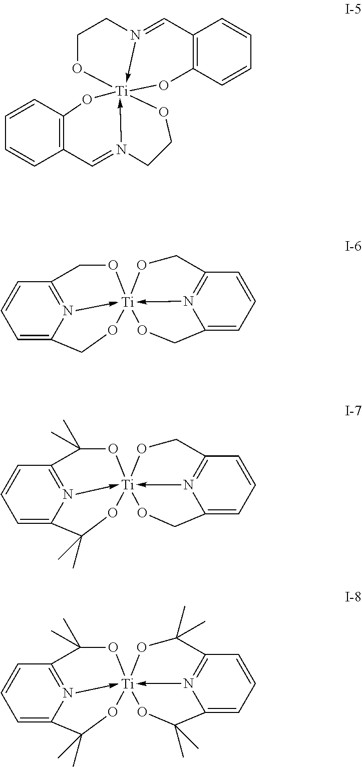

### <Evaluation>

### [Evaluation of Dielectric Loss Tangent (Df)]

Each of the resin compositions or comparative compositions prepared in each of Examples and Comparative Examples was applied onto a 12-inch silicon wafer by a spin coating method to form a resin composition layer. The silicon wafer on which the obtained resin composition layer was applied was dried on a hot plate at 100° C. for 5 minutes, thereby forming a resin composition layer having a uniform thickness on the silicon wafer, the thickness being described in the column of “Film thickness (m)” in the table. The resin composition layer on the silicon wafer was exposed over the entire surface using a stepper (Nikon NSR 2005 i9C) at an exposure energy of 500 mJ/cm², the exposed resin composition layer (resin layer) was heated at a temperature rising rate of 10° C./min in a nitrogen atmosphere, and heated at the temperature described in the column of “Cure temperature (° C.)” in the table for the time described in the column of “Cure time (min)” in the table, thereby obtaining a cured layer (resin layer) of the resin composition layer.

The cured layer (resin film) after curing was immersed in a 4.9% by mass hydrofluoric acid aqueous solution, and the cured film was peeled off from the silicon wafer.

For the film sample, a relative permittivity (Dk) and a dielectric loss tangent (Df) at 28 GHz were measured by a resonator perturbation method. The dielectric loss tangent (Df) was evaluated according to the following evaluation standard, and the evaluation results are described in the column of “Df” in the table.

### <Measurement Method>

Split cylinder resonator (CR-728)

### (Device Configuration)

Network analyzer: N5230A (manufactured by Keysight Technologies, Inc.)

### (Evaluation Standards)

Dielectric loss tangent (Df)
A: The dielectric loss tangent (Df) was less than 0.015.
B: The dielectric loss tangent (Df) was 0.015 to less than 0.02.
C: The dielectric loss tangent (Df) was 0.02 or more.

### [Evaluation of Metal Adhesiveness]

Each of the resin composition and the comparative composition prepared in each of Examples and Comparative Examples was applied respectively in a layer shape onto a copper substrate by a spin coating method to form a resin composition layer or a comparative composition layer. The copper substrate on which the obtained resin composition layer or comparative composition layer was formed was dried on a hot plate at 100° C. for 5 minutes, thereby obtaining a resin composition layer or comparative composition layer having a thickness described in the column of “Film thickness (m)” in the table, the thickness being uniform. The resin composition layer or the comparative composition layer on the copper substrate was exposed to i-rays using a photo mask in which a square non-masked portion having a square shape of 100 m square was formed at an exposure energy of 500 mJ/cm² using a stepper (Nikon NSR 2005 i9C), developed for 60 seconds with a developer described in the column of “Developer” in the table, and rinsed with propylene glycol monomethyl ether acetate (PGMEA) to obtain a resin layer having a square shape of 100 m square. Further, the resin layer (pattern) was formed by heating using a heating oven at the temperature described in the column of “Cure temperature (° C.)” in the table and the time described in the column of “Cure time (min)” in the table under a nitrogen atmosphere.

The resin layer of 100 m square on a copper substrate was subjected to the shearing force measurement in an environment of 25° C. and 65% relative humidity (RH) using a bond tester (Condor Sigma, manufactured by XYZTEC). It can be said that the larger the shearing force, the better the metal adhesiveness (the copper adhesiveness) of the cured film. In any of Examples and Comparative Examples, the shearing force was more than 30 gf.

[Evaluation of Adhesive Strength after High Temperature Storage-Test (HTS)]

The resin layer and the copper substrate were heated at the temperature described in the column of “Cure temperature (° C.)” in the table for the time described in the column of “Cure time (min)” in the table, and then allowed to stand in a constant-temperature tank at 175° C. for 1000 hours, and then the shearing force was measured according to the same evaluation method as the evaluation method in the evaluation of the metal adhesiveness described above, and the metal adhesiveness after heating was evaluated. The evaluation was performed according to the following evaluation standard from the measured shearing force, and the evaluation results are described in the column of “Reliability (post-HTS adhesion strength)” in the table. It can be said that the larger the shearing force, the better the metal adhesiveness (copper adhesiveness) of the cured film, and since the results of the post-HTS adhesion are excellent, it can be said that the cured film is unlikely to be peeled off from the metal even after a long period of time.

### (Evaluation Standards)

A: The shearing force was more than 30 gf.
B: The shearing force was more than 25 gf and 30 gf or less.
D: The shearing force was 25 gf or less.

Here, 1 gf is 0.00980665 N.

### [Breaking Elongation Rate]

In each of Examples and Comparative Examples, each resin composition or each comparative composition was applied in a layer shape onto a silicon wafer by a spin coating method to form a resin composition layer or a comparative composition layer. The silicon wafer to which the obtained resin composition layer or comparative composition layer was applied was dried on a hot plate at 100° C. for 5 minutes, thereby obtaining a resin composition layer or comparative composition layer having a uniform thickness described in the column of “Film thickness (μm)” in the table on the silicon wafer. The resin composition layer or comparative composition layer on the silicon wafer was exposed with an exposure energy of 500 mJ/cm² using a stepper (Nikon NSR 2005 i9C). The temperature of the exposed resin composition layer or comparative composition layer was raised at a temperature rising rate of 10° C./min in a nitrogen atmosphere using a hot plate, and after reaching the temperature described in the column of “Cure temperature (° C.)” in the table, the temperature was maintained for a time described in the column of “Cure time (min)” in the table, thereby obtaining a cured resin layer. The resin layer after curing was immersed in a 4.9% by mass hydrofluoric acid solution, and the resin layer was peeled off from the silicon wafer to obtain a resin film 1.

The resin film 1 was punched with a puncher to produce a film having a sample width of 10 mm and a sample length of 50 mm. The breaking elongation rate of the film was measured in accordance with JIS-K6251:2017 in an environment of 25° C. and a relative humidity (RH) of 65% using a tensile tester (Tensilon) at a crosshead speed of 300 mm/min in the longitudinal direction and the width direction of the film. The breaking elongation rate was calculated by Eb (%)=(Lb−L0)/L0×100 (Eb: elongation at the time of breaking, L0: length of test piece before test, Lb: length of test piece in case where test piece is cut). The evaluation was performed by measuring the breaking elongation rate in the longitudinal direction 10 times and using the arithmetic mean value of a total of 10 breaking elongation rates (Eb) as the index value. The evaluation was performed according to the following evaluation standards. It can be said that the larger the numerical value of the index value, the more excellent the breaking elongation. The evaluation results are described in the column of “Breaking elongation” in the table.

### —Evaluation Standards—

A: The index value was more than 60%.
B: The index value was more than 40% and 60% or less.
C: The index value was 40% or less.

### Example 101

The resin composition used in Example 1 was applied in a layer shape onto a surface of a thin copper layer of the resin base material on the surface of which the thin copper layer was formed, by a spin coating method, dried at 100° C. for 5 minutes, and after forming a photosensitive film having a thickness of 20 m, exposure was carried out using a stepper (NSR1505 i6, manufactured by Nikon Corporation). Exposure was carried out through a mask (a binary mask in which the pattern is a pattern of 1:1 line and space and the line width is 10 μm) at a wavelength of 365 nm. After the exposure, the layer was developed with cyclopentanone for 2 minutes and rinsed with PGMEA for 30 seconds to obtain a layer pattern.

Next, the temperature was raised at a temperature rising rate of 10° C./min in a nitrogen atmosphere, and after the temperature reached 230° C., the temperature was maintained at 230° C. for 180 minutes, thereby forming an interlayer insulating film for a re-distribution layer. This interlayer insulating film for a re-distribution layer was excellent in insulating properties.

In addition, in a case where a semiconductor device was manufactured using this interlayer insulating film for a re-distribution layer, it has been confirmed that the semiconductor device operates without any problem.

What is claimed is:

1. A resin composition comprising:
a resin which includes a repeating unit represented by Formula (1-1), includes a group having an ethylenically unsaturated bond, and has a content of a phenolic hydroxyl group of 0.250 mmol/g or less; and
a polymerizable compound having an ethylenically unsaturated bond, 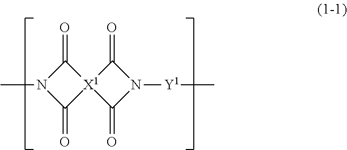
in Formula (1-1), X¹ represents a tetravalent organic group, and Y¹ represents a divalent organic group.

2. The resin composition according to claim 1,
wherein the resin is a resin including a repeating unit represented by Formula (1-2) as the repeating unit represented by Formula (1-1), 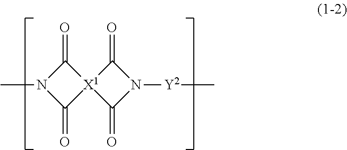
in Formula (1-2), X¹ represents a tetravalent organic group, and Y² represents a group including a group represented by Formula (2-1), 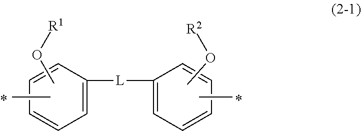
in Formula (2-1), R¹ and R² each independently represent a group having an ethylenically unsaturated bond, L represents a single bond or a divalent linking group which does not have an imide bond, and *'s each represent a bonding site to another structure.

3. The resin composition according to claim 1,
wherein the resin includes a group having a vinylphenyl group as the group having an ethylenically unsaturated bond.

4. A resin composition comprising:
a resin which includes a repeating unit represented by Formula (1-3) and a vinylphenyl group; and
a polymerizable compound having an ethylenically unsaturated bond, 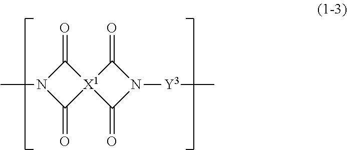
in Formula (1-3), X¹ represents a tetravalent organic group, and Y³ represents a group including a group represented by Formula (2-2), 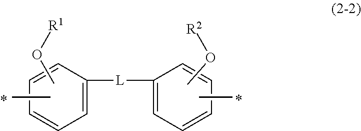
in Formula (2-2), R¹ and R² each independently represent a group having an ethylenically unsaturated bond, L represents a single bond, —C(CH₃)₂—, or —C(CF₃)₂—, and *'s each represent a bonding site to another structure.

5. The resin composition according to claim 1,
wherein the resin has a structure represented by any of Formulae (3-1) to (3-3), 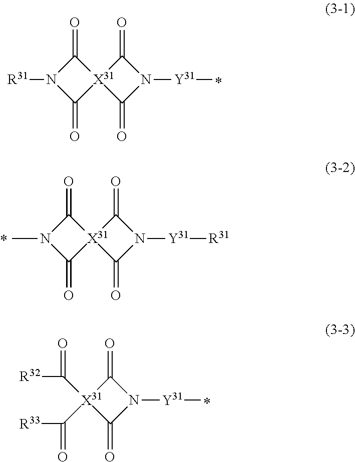
in Formula (3-1), X³¹ represents a tetravalent organic group, Y³¹ represents a divalent organic group, R³¹ represents a group having an ethylenically unsaturated bond, and * represents a bonding site to another structure,
in Formula (3-2), X³¹ represents a tetravalent organic group, Y³¹ represents a divalent organic group, R³¹ represents a group having an ethylenically unsaturated bond, and * represents a bonding site to another structure,
in Formula (3-3), X³¹ represents a tetravalent organic group, Y³¹ represents a divalent organic group, R³² and R³³ each independently represent —OH or a monovalent organic group, at least one of R³² or R³³ is a group having an ethylenically unsaturated bond, and * represents a bonding site to another structure.

6. The resin composition according to claim 1,
wherein the resin is a resin in which a rate of change in an imide group value calculated by Expression before and after heating at 350° C. and 1 atm for 1 hour is 25% or less,
$$rate of change ( \% ) = ( Im 2 - Im 1 ) \times 10 \; 0 / Im 1 ,$$
Im1: an imide group value before heating (mmol/g),
Im2: an imide group value (mmol/g) after heating at 350° C. and 1 atm for 1 hour.

7. The resin composition according to claim 1,
wherein X¹ is an organic group including a structure obtained by removing two or more hydrogen atoms from a structure represented by any of Formulae (V-1) to (V-4), 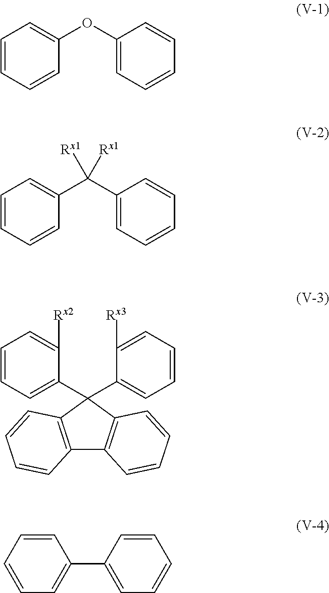
in Formula (V-2), R^{X1}'s each independently represent a hydrogen atom, an alkyl group, or a halogenated alkyl group,
in Formula (V-3), R^{X2} and R^{X3} each independently represent a hydrogen atom or a substituent, and R^{X2} and R^{X3} may be bonded to each other to form a ring structure.

8. The resin composition according to claim 1, further comprising:
an azole compound.

9. A diamine compound represented by Formula (4-1), 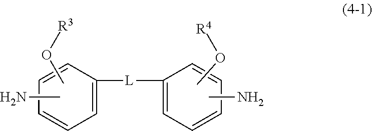
in Formula (4-1), R³ and R⁴ each independently represent a group having an ethylenically unsaturated bond, at least one of R³ or R⁴ has an aromatic hydrocarbon group, and L represents a single bond, —C(CH₃)₂—, or —C(CF₃)₂—.

10. The diamine compound according to claim 9,
wherein R³ and R⁴ each independently have a vinylphenyl group.

11. A resin composition comprising:
a resin which is a reaction product of the diamine compound according to claim 9 and a compound having two or more carboxy groups and carboxylic acid anhydride groups in total or a derivative thereof; and
a polymerizable compound having an ethylenically unsaturated bond.

12. The resin composition according to claim 1,
wherein the resin composition is used for forming an interlayer insulating film for a re-distribution layer.

13. A cured substance obtained by curing the resin composition according to claim 1.

14. A laminate comprising:
two or more layers consisting of the cured substance according to claim 13; and
a metal layer provided between any layers consisting of the cured substance.

15. A manufacturing method for a cured substance, comprising:
a film forming step of applying the resin composition according to claim 1 onto a base material to form a film.

16. The manufacturing method for a cured substance according to claim 15, further comprising:
an exposure step of selectively exposing the film; and
a development step of developing the film using a developer to form a pattern.

17. The manufacturing method for a cured substance according to claim 15, further comprising:
a heating step of heating the film at 50° C. to 450° C.

18. A manufacturing method for a laminate, comprising:
the manufacturing method for a cured substance according to claim 15.

19. A manufacturing method for a semiconductor device, comprising:
the manufacturing method for a cured substance according to claim 15.

20. A semiconductor device comprising:
the cured substance according to claim 13.
